(19) United States
(12) Patent Application Publication
(10) Pub. No.: US 2024/0398701 A1
(43) Pub. Date: Dec. 5, 2024

(54) MANUFACTURING METHODS FOR GASTRIC RESIDENCE SYSTEMS

(71) Applicant: Lyndra Therapeutics, Inc., Watertown, MA (US)

(72) Inventors: Rosemary KANASTY, Somerville, MA (US); Tyler GRANT, Burlington, MA (US); Craig SIMSES, Quincy, MA (US); Carmi MANDELKERN, Cambridge, MA (US); Juan Jaramillo MONTEZCO, Somerville, MA (US); Sonia HOLAR, Cambridge, MA (US)

(21) Appl. No.: 18/731,125

(22) Filed: May 31, 2024

Related U.S. Application Data
(60) Provisional application No. 63/506,039, filed on Jun. 2, 2023.

(51) Int. Cl. A61K 9/00; A61K 9/48

(52) U.S. Cl. CPC A61K 9/0065; A61K 9/4833; A61K 9/4866

(57) ABSTRACT

A gastric residence system comprises one or more retention members comprising: at least one drug eluting component; and at least one laser linker component laser welded to the at least one drug eluting component.

### CROSS-REFERENCE TO RELATED APPLICATIONS

This application claims priority benefit of U.S. Provisional Patent Application No. 63/506,039 filed Jun. 2, 2023. The entire contents of that application are hereby incorporated by reference herein.

### FIELD

The present disclosure relates generally to gastric residence systems, and more specifically to manufacturing methods for gastric residence systems.

### BACKGROUND

Gastric residence systems are delivery systems for therapeutic agents which remain in the stomach for days to weeks, or even over longer periods, during which time drugs or other agents can elute from the systems for absorption in the gastrointestinal tract.

Gastric residence systems can be administered to a patient using capsules which are swallowed or introduced into the stomach of the patient by an alternate method (e.g., via a feeding tube or a gastric tube). Upon dissolution of a capsule in the stomach, a gastric residence system may expand or unfold to a size which remains in the stomach and resists passage through the pylorus over a desired gastric residence period. Throughout the desired residence period, the system elutes one or more agents (e.g., drugs) at a desired rate. At the end of the residence period, the system passes through the pylorus and is eliminated from the patient. If the system passes through the pylorus before the end of the desired residence period, however, the one or more agents may not be delivered to the patient as intended. Accordingly, gastric residence systems need to be manufactured such that the system stays intact until the end of the desired residence period.

Gastric residence systems often include multiple components which serve different functions. For example, a gastric residence system may include an elastomeric component which allows the system to be compacted, a drug eluting component which elutes an active pharmaceutical ingredient at a desired rate, time-dependent and enteric components which help the system break apart at the end of the desired residence time, and inactive components which help maintain the size and shape of the system. Each of these components may comprise a variety of different polymers with different characteristics. The polymers used in these components may be challenging to adhere using conventional manufacturing methods due to their different compositions, properties, and functions. Accordingly, there is a need for improved methods of manufacturing gastric residence systems.

### SUMMARY

As described, gastric residence systems may comprise multiple different components which serve different functions, such as elastomeric components, drug eluting components, time-dependent and enteric components, and/or inactive components. Joining these different components while preserving each of their specific functionalities using conventional manufacturing methods is challenging. For example, some methods involve overmolding multiple components of a gastric residence system onto one another. However, this may make the system too large to fit in a conventional capsule for administration and may hamper the functionality of the various components. Components can alternatively be joined using adhesives, but the adhesives used must be safe for human consumption since the systems described herein are designed to be swallowed by a patient. Using adhesives also introduces additional components to the system, which introduces additional failure points.

Some existing manufacturing techniques do not introduce additional components to the system, such as infrared welding or hot plate welding. However, infrared welding and hot plate welding introduce challenges of their own. For example, certain components of gastric residence systems (e.g., drug eluting components) may be temperature-sensitive. The heat provided during infrared welding and hot plate welding may not be concentrated at the interface between components and thus may melt more of the components than is necessary to join them, causing damage to the temperature-sensitive components. Furthermore, infrared welding and hot plate welding typically require the polymers to be welded together to be similar (e.g., in structure or composition) in order to produce strong welds. However, the components used in the gastric residence systems described herein include various polymers with different functionalities and characteristics.

Provided herein are improved manufacturing techniques which use laser welding to adhere components of gastric residence systems. Laser welding uses laser energy to heat an interface between components such that the components melt and fuse together at the interface. The links created by laser welding may be stronger than those created by other polymer joining techniques, resulting in stronger gastric residence systems and enhanced protection against breakage and premature passage through the pylorus. Laser welding may also be advantageous because it can be used to effectively weld components with different polymer compositions while allowing the different components to maintain their specific functionalities. Furthermore, laser welding uses a focused energy beam to weld components, which makes it easier to avoid damaging heat-sensitive components, such as drug eluting components.

A gastric residence system comprises: one or more retention members comprising: at least one drug eluting component; and at least one laser linker component laser welded to the at least one drug eluting component.

In some embodiments, the one or more retention members are attached to an elastomeric component. In some embodiments, the elastomeric component is overmolded onto a first portion of at least one intercomponent anchor. In some embodiments, a laser linker component is overmolded onto a second portion of the at least one intercomponent anchor. In some embodiments, a first retention member is attached to the elastomeric component via the overmolded laser linker component. In some embodiments, the at least one laser linker component comprises an inactive component. In some embodiments, the inactive component comprises polycaprolactone. In some embodiments, the inactive component further comprises bismuth subcarbonate. In some embodiments, the inactive component further comprises copovidone. In some embodiments, the inactive component further comprises a poloxamer. In some embodiments, the inactive component further comprises a colorant. In some embodiments, the at least one laser linker component comprises an enteric disintegrating matrix. In some embodiments, the enteric disintegrating matrix comprises polycaprolactone. In some embodiments, the enteric disintegrating matrix further comprises HPMCAS. In some embodiments, the enteric disintegrating matrix further comprises a poloxamer. In some embodiments, the at least one laser linker component comprises a time-dependent disintegrating matrix. In some embodiments, the time-dependent disintegrating matrix comprises polycaprolactone. In some embodiments, the time-dependent disintegrating matrix further comprises poly(ethylene oxide). In some embodiments, the time-dependent disintegrating matrix further comprises 50/50 DL-Lactide/Glycolide copolymer. In some embodiments, the time-dependent disintegrating matrix further comprises ferrosoferric oxide. In some embodiments, the drug eluting component comprises polycaprolactone. In some embodiments, the drug eluting component further comprises an active pharmaceutical ingredient. In some embodiments, the active pharmaceutical ingredient comprises one or more of meloxicam, escitalopram, citalopram, clopidogrel, prednisone, aripiprazole, risperidone, buprenorphine, naloxone, montelukast, memantine, digoxin, tamsulosin, ezetimibe, colchicine, loratadine, cetirizine, loperamide, omeprazole, entecavir, doxycycline, ciprofloxacin, azithromycin, antimalarial agents, levothyroxine, methadone, varenicline, contraceptives, stimulants, or nutrients. In some embodiments, the drug eluting component further comprises copovidone. In some embodiments, the drug eluting component further comprises a poloxamer. In some embodiments, the drug eluting component further comprises vitamin E succinate. In some embodiments, the drug eluting component further comprises silicon dioxide. In some embodiments, the drug eluting component further comprises a colorant. In some embodiments, a difference between a melt flow index of the at least one laser linker component and a melt flow index of the at least one drug eluting component is greater than 10%. In some embodiments, a difference between a melt flow index of the at least one laser linker component and a melt flow index of the at least one drug eluting component is less than 50%. In some embodiments, a polycaprolactone content of the at least one laser linker component is at least 30% by weight. In some embodiments, a polycaprolactone content of the at least one laser linker component is at least 40% by weight. In some embodiments, a polycaprolactone content of the at least one drug eluting component is at least 30% by weight. In some embodiments, a polycaprolactone content of the at least one drug eluting component is at least 40% by weight. In some embodiments, a polycaprolactone content of the at least one laser linker component is less than 50% by weight. In some embodiments, a polycaprolactone content of the at least one laser linker component is less than 65% by weight. In some embodiments, a polycaprolactone content of the at least one laser linker component is less than 75% by weight. In some embodiments, a polycaprolactone content of the at least one drug eluting component is less than 50% by weight. In some embodiments, a polycaprolactone content of the at least one drug eluting component is less than 65% by weight. In some embodiments, a polycaprolactone content of the at least one drug eluting component is less than 75% by weight. In some embodiments, a melting temperature of the at least one drug eluting component is within 1-75° C. of a melting temperature of the at least one laser linker component. In some embodiments, a melting temperature of the at least one drug eluting component is within 5-50° C. of a melting temperature of the at least one laser linker component. In some embodiments, a width of a melt zone between laser welded components of the one or more retention members is between 0.5 mm and 5 mm. In some embodiments, a width of a melt zone between laser welded components of the one or more retention members is between 1 mm and 3 mm. In some embodiments, a depth of a melt zone between laser welded components of the one or more retention members is at least 90% of a depth of an interface between the laser welded components. In some embodiments, a depth of a melt zone between laser welded components of the one or more retention members is at least 95% of a depth of an interface between the laser welded components. In some embodiments, the gastric residence system receives a score of at least 7 when using the window-cyclic funnel test. In some embodiments, the gastric residence system receives a score of at least 10 when using the window-cyclic funnel test. In some embodiments, the gastric residence system is configured to be in a stressed configuration during administration and is configured to assume an open configuration when in a patient's stomach.

A method of making a gastric residence system comprising one or more retention members comprising at least one drug eluting component and at least one laser linker component comprises: laser welding the at least one drug eluting component to the at least one laser linker component.

In some embodiments, the method further comprises: placing the at least one laser linker component and the at least one drug eluting component in a laser welding holder prior to laser welding. In some embodiments, the method further comprises: applying a radial force which presses the at least one drug eluting component against the at least one laser linker component. In some embodiments, the radial force is between 5 N and 200 N. In some embodiments, the radial force is between 10 N and 50 N. In some embodiments, the method further comprises: applying a downward force which presses the at least one drug eluting component and the at least one laser linker component against the laser welding holder. In some embodiments, the vertical force is between 100 N and 5000 N. In some embodiments, the vertical force is between 200 N and 2800 N. In some embodiments, laser welding is performed using a laser having a wavelength between 0.7 μm and 2.5 μm. In some embodiments, laser welding is performed using a laser having a wavelength between 1.9 μm and 2.0 μm. In some embodiments, laser welding is performed using a laser having a Gaussian energy profile or a top hat energy profile. In some embodiments, laser welding is performed using a laser having a beam diameter between 0.5 mm and 5 mm. In some embodiments, laser welding is performed using a laser having a beam diameter between 1 mm and 3 mm. In some embodiments, laser welding is performed in an environment with less than 40% humidity. In some embodiments, laser welding is performed in an environment with less than 25% humidity. In some embodiments, laser welding is performed in an environment with at least 10% humidity. In some embodiments, laser welding is performed in an environment with at least 15% humidity. In some embodiments, the method further comprises: laser welding the one or more retention members to an elastomeric component, wherein the elastomeric component comprises one or more laser linker components configured to be laser welded to the one or more retention members. In some embodiments, the gastric residence system comprises at least two retention members laser welded to the elastomeric component. In some embodiments, the gastric residence system comprises at least three retention members laser welded to the elastomeric component. In some embodiments, the gastric residence system comprises at least four retention members laser welded to the elastomeric component. In some embodiments, the gastric residence system comprises at least five retention members laser welded to the elastomeric component. In some embodiments, the gastric residence system comprises at least six retention members laser welded to the elastomeric component. In some embodiments, laser welding the at least one drug eluting component to the at least one laser linker component comprises laser welding along a repetitive path connecting corresponding interfaces between the at least one drug eluting component and the at least one linker on each retention member. In some embodiments, the repetitive path is a circular path. In some embodiments, laser welding the at least one drug eluting component to the at least one laser linker component comprises laser welding back and forth along an interface between the at least one drug eluting component and the at least one laser linker component.

A laser welding system comprises: a laser welding holder configured to hold pieces of a gastric residence system in an assembled order; at least one radial pressure piston configured to apply a radial force to the gastric residence system in a direction transverse to seams between each piece; at least one vertical pressure piston configured to apply a downward force to the gastric residence system in a direction parallel to the seams between each piece; and at least one laser configured to weld the seams between each piece of the gastric residence system.

In some embodiments, the laser welding holder comprises aluminum or stainless steel. In some embodiments, the laser welding holder is plated with nickel. In some embodiments, the laser welding holder comprises grooves sized and shaped to receive the pieces of the gastric residence system. In some embodiments, a top surface of the laser welding holder comprises a layer of silicone. In some embodiments, a top surface of the laser welding holder comprises a layer of polytetrafluoroethylene-coated glass. In some embodiments, the laser welding system further comprises a cover configured to hold the components of the gastric residence system in place during laser welding. In some embodiments, the cover comprises silicone or polytetrafluoroethylene-coated glass. In some embodiments, the laser welding system further comprises a top layer on top of the cover. In some embodiments, the top layer comprises quartz glass. In some embodiments, the radial force applied by the at least one radial pressure piston is between 5 and 200 N. In some embodiments, the radial force applied by the at least one radial pressure piston is between 10 and 50 N. In some embodiments, the downward force applied by the at least one vertical pressure piston is between 100 and 5000 N. In some embodiments, the downward force provided by the at least one vertical pressure piston is between 200 and 2800 N. In some embodiments, the at least one laser has a wavelength between 0.7 μm and 2.5 μm. In some embodiments, the at least one laser has a wavelength between 1.9 μm and 2.0 μm. In some embodiments, the at least one laser has a Gaussian energy profile or a top hat energy profile. In some embodiments, the at least one laser has a beam diameter between 0.5 mm and 5 mm. In some embodiments, the at least one laser has a beam diameter between 1 mm and 3 mm.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The invention will now be described, by way of example only, with reference to the accompanying drawings, in which:

FIG. 1A shows a gastric residence system in an open configuration.

FIG. 1B shows a gastric residence system in a folded configuration.

FIG. 2 shows a gastric residence system comprising a plurality of retention members and the bent geometry a gastric residence system can assume most easily when compressed by forces such as gastric contractions.

FIGS. 3A-3C show various methods by which a gastric residence system may pass through a pylorus before the gastric residence time has elapsed.

FIG. 4A shows an exemplary Gaussian energy profile for a laser beam.

FIG. 4B shows an exemplary top hat energy profile for a laser beam.

FIG. 5 shows an exemplary laser welded gastric residence system.

FIGS. 6A-6B show exemplary energy heat maps produced by laser welding a gastric residence system.

FIGS. 7A-7B show exemplary energy heat maps produced by wobbling a laser beam around a circular path.

FIGS. 8A-8B illustrate an exemplary laser welding assembly.

FIG. 9 illustrates an exemplary laser welding holder.

FIG. 10 illustrates an exemplary gastric residence system.

FIG. 11 illustrates a method for quantifying the weld strength of gastric residence systems.

FIG. 12 shows tensile testing results for gastric residence systems manufactured using laser welding and infrared welding.

FIG. 13 shows the absorbance of various components of a laser welded gastric residence system.

FIG. 14 shows the absorbance of various components of a laser welded gastric residence system.

FIG. 15 shows the results of window-cyclic funnel testing versus total stellate energy for a laser welded gastric residence system.

FIG. 16 shows the melting temperatures and pressures of various components of a laser welded gastric residence system.

### DETAILED DESCRIPTION

Described herein are manufacturing methods for gastric residence systems. Gastric residence systems are designed to reside in the gastrointestinal tract of a patient for a predetermined residence time. After the residence time elapses, the gastric residence system breaks down into several pieces small enough to pass through the pylorus. If the gastric residence system breaks down prematurely, the therapeutic agent included in the gastric residence system may not be administered to the patient as intended.

Accordingly, gastric residence systems need to be manufactured in a manner that ensures the systems do not break apart prematurely. Provided herein are manufacturing methods for gastric residence systems which use laser welding to thermally join components of a gastric residence system.

Laser welding uses laser energy to heat an interface between components such that the components melt and fuse together at the interface. Exemplary gastric residence systems manufactured using the laser welding techniques described herein may include one or more retention members. Each retention member may include at least one drug eluting component and at least one laser linker component laser welded to the drug eluting component. Laser linker components may include time-dependent or enteric disintegrating matrices or inactive components and may be used both to control the properties of the gastric residence system (e.g., to achieve a predetermined gastric residence time or to maintain the size and shape of the gastric residence system).

Joining these various components of a gastric residence system while preserving their functionality and integrity can be challenging. For example, overmolding can be used to join components; however, this may make the system too large to fit in a conventional capsule for administration and may hamper the functionality of the various components. Adhesives can also be used to join components, but the adhesives used must be safe for human consumption, and using adhesive components introduces additional failure points into the system. Thermal joining techniques (e.g., infrared welding or hot plate welding) can also be used, but these methods can damage heat-sensitive components and may not be effective unless the components to be welded comprise polymers with relatively similar properties.

The laser welding techniques provided herein may remedy one or more of the above-identified issues. Laser welding may be used to effectively weld components with different polymer compositions while allowing the different components to maintain their specific properties and functionalities. Additionally, laser welding uses a focused energy beam to weld components, which can help preserve the integrity and functionality of heat-sensitive components. The connections created by laser welding may also be stronger than those produced using conventional techniques to join gastric residence system components, which can lead to enhanced protection against breakage of the systems and improved gastric retention.

### Definitions

As used herein, “gastric residence system” is a dosage form comprising an agent and is configured to be administered to a patient in a folded configuration. A “gastric residence dosage form” comprises a folded gastric residence system and is configured to hold the gastric residence system in a folded configuration until deployment. For example, a gastric residence dosage form may comprise a capsule and/or a capsule coating according to those described in U.S. Appln. No. 62/821,352 titled “Capsules and Capsule Coatings for Gastric Residence Dosage Forms” and/or U.S. Appln. No. 62/821,361 titled “Coatings for Gastric Residence Forms.”

A “carrier polymer” is a polymer suitable for blending with an agent, such as a drug, for use in the invention.

An “agent” is any substance intended for therapeutic, diagnostic, or nutritional use in a patient, individual, or subject. Agents include, but are not limited to, drugs, nutrients, vitamins, and minerals.

A “dispersant” is defined as a substance which aids in the minimization of particle size of agent and the dispersal of agent particles in the carrier polymer matrix. That is, the dispersant helps minimize or prevent aggregation or flocculation of particles during fabrication of the systems. Thus, the dispersant has anti-aggregant activity and anti-flocculant activity, and helps maintain an even distribution of agent particles in the carrier polymer matrix.

An “excipient” is any substance added to a formulation of an agent that is not the agent itself. Excipients include, but are not limited to, binders, coatings, diluents, disintegrants, emulsifiers, flavorings, glidants, lubricants, and preservatives. The specific category of dispersant falls within the more general category of excipient.

An “elastic polymer” or “elastomer” (also referred to as a “tensile polymer”) is a polymer that is capable of being deformed by an applied force from its original shape for a period of time, and which then substantially returns to its original shape once the applied force is removed.

A “coupling polymer” is a polymer suitable for coupling any other polymers together, such as coupling a first carrier polymer-agent component to a second carrier polymer-agent component. Coupling polymers typically form the linker regions between other components.

A “time-dependent polymer” or “time-dependent coupling polymer” is a polymer that degrades in a time-dependent manner when a gastric residence system is deployed in the stomach. A time-dependent polymer is typically not affected by the normal pH variations in the stomach.

“Approximately constant plasma level” refers to a plasma level that remains within a factor of two of the average plasma level (that is, between 50% and 200% of the average plasma level) measured over the period that the gastric residence system is resident in the stomach.

“Biocompatible,” when used to describe a material or system, indicates that the material or system does not provoke an adverse reaction, or causes only minimal, tolerable adverse reactions, when in contact with an organism, such as a human. In the context of the gastric residence systems, biocompatibility is assessed in the environment of the gastrointestinal tract.

A “patient,” “individual,” or “subject” refers to a mammal, preferably a human or a domestic animal such as a dog or cat. In a most preferred embodiment, a patient, individual, or subject is a human.

The “diameter” of a particle as used herein refers to the longest dimension of a particle.

“Treating” a disease or disorder with the systems and methods disclosed herein is defined as administering one or more of the systems disclosed herein to a patient in need thereof, with or without additional agents, in order to reduce or eliminate either the disease or disorder, or one or more symptoms of the disease or disorder, or to retard the progression of the disease or disorder or of one or more symptoms of the disease or disorder, or to reduce the severity of the disease or disorder or of one or more symptoms of the disease or disorder.

“Suppression” of a disease or disorder with the systems and methods disclosed herein is defined as administering one or more of the systems disclosed herein to a patient in need thereof, with or without additional agents, in order to inhibit the clinical manifestation of the disease or disorder, or to inhibit the manifestation of adverse symptoms of the disease or disorder. The distinction between treatment and suppression is that treatment occurs after adverse symptoms of the disease or disorder are manifest in a patient, while suppression occurs before adverse symptoms of the disease or disorder are manifest in a patient. Suppression may be partial, substantially total, or total. Because some diseases or disorders are inherited, genetic screening can be used to identify patients at risk of the disease or disorder. The systems and methods of the invention can then be used to treat asymptomatic patients at risk of developing the clinical symptoms of the disease or disorder, in order to suppress the appearance of any adverse symptoms.

“Therapeutic use” of the systems disclosed herein is defined as using one or more of the systems disclosed herein to treat a disease or disorder, as defined above. A “therapeutically effective amount” of a therapeutic agent, such as a drug, is an amount of the agent, which, when administered to a patient, is sufficient to reduce or eliminate either a disease or disorder or one or more symptoms of a disease or disorder, or to retard the progression of a disease or disorder or of one or more symptoms of a disease or disorder, or to reduce the severity of a disease or disorder or of one or more symptoms of a disease or disorder. A therapeutically effective amount can be administered to a patient as a single dose, or can be divided and administered as multiple doses.

“Prophylactic use” of the systems disclosed herein is defined as using one or more of the systems disclosed herein to suppress a disease or disorder, as defined above. A “prophylactically effective amount” of an agent is an amount of the agent, which, when administered to a patient, is sufficient to suppress the clinical manifestation of a disease or disorder, or to suppress the manifestation of adverse symptoms of a disease or disorder. A prophylactically effective amount can be administered to a patient as a single dose, or can be divided and administered as multiple doses.

A “flexural modulus” of a material is an intrinsic property of a material computed as the ratio of stress to strain in flexural deformation of the material as measured by a 3-point bending test. Although the linkers are described herein as being components of the gastric residence system, the flexural modulus of the material of the polymeric material may be measured in isolation. For example, the polymeric linker in the gastric residence system may be too short to measure the flexural modulus, but a longer sample of the same material may be used to accurately determine the flexural modulus. The longer sample used to measure the flexural modulus should have the same cross-sectional dimensions (shape and size) as the polymeric linker used in the gastric residence system. The flexural modulus is measured using a 3-point bending test in accordance with the ASTM standard 3-point bending test (ASTM D790) using a 10 mm distance between supports and further modified to accommodate materials with non-rectangular cross-sections. The longest line of symmetry for the cross section of the polymeric linker should be positioned vertically, and the flexural modulus should be measured by applying force downward. If the longest line of symmetry for the cross section of the polymeric linker is perpendicular to a single flat edge, the single flat edge should be positioned upward. If the cross-section of the polymeric linker is triangular, the apex of the triangle should be faced downward. As force is applied downward, force and displacement are measured, and the slope at the linear region is obtained to calculate the flexural modulus.

As used herein, the singular forms “a,” “an,” and “the” are intended to include the plural forms as well, unless the context clearly indicates otherwise. It is also to be understood that the term “and/or” as used herein refers to and encompasses any and all possible combinations of one or more of the associated listed items. It is further to be understood that the terms “includes, “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

When numerical values are expressed herein using the term “about” or the term “approximately,” it is understood that both the value specified, as well as values reasonably close to the value specified, are included. For example, the description “about 50° C.” or “approximately 50° C.” includes both the disclosure of 50° C. itself, as well as values close to 50° C. Thus, the phrases “about X” or “approximately X” include a description of the value X itself. If a range is indicated, such as “approximately 50° C. to 60° C.” or “about 50° C. to 60° C.,” it is understood that both the values specified by the endpoints are included, and that values close to each endpoint or both endpoints are included for each endpoint or both endpoints; that is, “approximately 50° C. to 60° C.” (or “about 50° C. to 60° C.”) is equivalent to reciting both “50° C. to 60° C.” and “approximately 50° C. to approximately 60° C.” (or “about 50° C. to 60° C.”).

This application discloses several numerical ranges in the text and figures. The numerical ranges disclosed inherently support any range or value within the disclosed numerical ranges, including the endpoints, even though a precise range limitation is not stated verbatim in the specification because this disclosure can be practiced throughout the disclosed numerical ranges.

With respect to numerical ranges disclosed in the present description, any disclosed upper limit for a component may be combined with any disclosed lower limit for that component to provide a range (provided that the upper limit is greater than the lower limit with which it is to be combined). Each of these combinations of disclosed upper and lower limits are explicitly envisaged herein. For example, if ranges for the amount of a particular component are given as 10% to 30%, 10% to 12%, and 15% to 20%, the ranges 10% to 20% and 15% to 30% are also envisaged, whereas the combination of a 15% lower limit and a 12% upper limit is not possible and hence is not envisaged.

Unless otherwise specified, percentages of ingredients in compositions are expressed as weight percent, or weight/weight percent. It is understood that reference to relative weight percentages in a composition assumes that the combined total weight percentages of all components in the composition add up to 100. It is further understood that relative weight percentages of one or more components may be adjusted upwards or downwards such that the weight percent of the components in the composition combine to a total of 100, provided that the weight percent of any particular component does not fall outside the limits of the range specified for that component.

Some embodiments described herein are recited as “comprising” or “comprises” with respect to their various elements. In alternative embodiments, those elements can be recited with the transitional phrase “consisting essentially of” or “consists essentially of” as applied to those elements. In further alternative embodiments, those elements can be recited with the transitional phrase “consisting of” or “consists of” as applied to those elements. Thus, for example, if a composition or method is disclosed herein as comprising A and B, the alternative embodiment for that composition or method of “consisting essentially of A and B” and the alternative embodiment for that composition or method of “consisting of A and B” are also considered to have been disclosed herein. Likewise, embodiments recited as “consisting essentially of” or “consisting of” with respect to their various elements can also be recited as “comprising” as applied to those elements. Finally, embodiments recited as “consisting essentially of” with respect to their various elements can also be recited as “consisting of” as applied to those elements, and embodiments recited as “consisting of” with respect to their various elements can also be recited as “consisting essentially of” as applied to those elements.

When a composition or system is described as “consisting essentially of” the listed elements, the composition or system contains the elements expressly listed, and may contain other elements which do not materially affect the condition being treated (for compositions for treating conditions), or the properties of the described system (for compositions comprising a system). However, the composition or system either does not contain any other elements which do materially affect the condition being treated other than those elements expressly listed (for compositions for treating systems) or does not contain any other elements which do materially affect the properties of the system (for compositions comprising a system); or, if the composition or system does contain extra elements other than those listed which may materially affect the condition being treated or the properties of the system, the composition or system does not contain a sufficient concentration or amount of those extra elements to materially affect the condition being treated or the properties of the system. When a method is described as “consisting essentially of” the listed steps, the method contains the steps listed, and may contain other steps that do not materially affect the condition being treated by the method or the properties of the system produced by the method, but the method does not contain any other steps which materially affect the condition being treated or the system produced other than those steps expressly listed.

This disclosure provides several embodiments. It is contemplated that any features from any embodiment can be combined with any features from any other embodiment where possible. In this fashion, hybrid configurations of the disclosed features are within the scope of the present invention.

### General Principles of Gastric Residence Systems

Provided herein are gastric residence systems and methods of manufacturing said systems. Gastric residence systems are designed to be administered to a stomach of a patient, either by swallowing or other method of administration (e.g., feeding tube or gastric tube). Once a gastric residence system is in place in the stomach, the system remains in the stomach for the desired residence period (e.g., three days, seven days, two weeks, etc.). During the residence period, the system resists passage through the pylorus, which separates the stomach and the small intestine. The system releases an agent (e.g., an active pharmaceutical ingredient or drug) into the stomach over the residence period at a controlled rate of release. While residing in the stomach, the system may not interfere with the normal passage of food or other gastric contents. Once the desired residence period has elapsed, the system may pass through the pylorus and be eliminated from the patient. If the system prematurely passes from the stomach into the small intestine, it does not cause intestinal obstruction, and again is readily eliminated from the patient.

To administer a gastric residence system to a patient, the gastric residence system may be folded into a configuration small enough to be swallowed or otherwise administered. In some embodiments, the folded gastric residence system is retained in a capsule or other container which can be swallowed by the patient or otherwise administered. In some embodiments, a capsule may comprise at least one of gelatin, hydroxypropyl methylcellulose, or pullulan.

In some embodiments, the folded gastric residence system may also be secured by a dissolvable retaining band or sleeve that can prevent premature deployment of the gastric residence system in case of a failure of the capsule or other container. A gastric residence system folded and retained in a folded configuration with a sleeve or band may be encapsulated by a capsule. In some embodiments, a sleeve or band may comprise at least one of gelatin, hydroxypropyl methylcellulose, or pullulan.

Once the capsule or other container storing the gastric residence system reaches the stomach of a patient, the capsule or container dissolve and release the folded gastric retention system. Upon release, the gastric retention system may unfold and assume an open configuration. The open configuration may be a stellate shape. The dimensions of the gastric residence system in the open configuration are, when left unaltered, suitable to prevent passage of the gastric residence system through the pylorus for the desired residence period.

While in the stomach, the gastric residence system is compatible with digestion and other normal functioning of the stomach or gastrointestinal tract. The gastric residence system does not interfere with or impede the passage of chyme (partially digested food) or other gastric contents which exit the stomach through the pylorus into the duodenum.

The gastric residence system releases an agent (e.g., while in the stomach). The gastric residence system may comprise a plurality of polymer-agent components. In one embodiment, the polymer-agent components comprise a carrier polymer, a dispersant, and an agent (e.g., an active pharmaceutical ingredient). In another embodiment, the polymer-agent components comprise a carrier polymer and an agent. The plurality of polymer-agent components are linked together by one or more laser linker components and/or one or more elastomeric components. Agent is eluted from the carrier polymer-agent components into the gastric fluid of the patient over the desired residence time of the system. Release of the agent is controlled by appropriate formulation of the carrier polymer-agent components, including by the use of the dispersant in formulation of the carrier polymer-agent components, and by milling of the agent to particles of desired size prior to blending the agent with the carrier polymer and dispersant. In addition, coatings can be applied to outer surfaces of the gastric residence system. The coatings may include additional agents or agents that can affect the release of agents or the residence period of the gastric residence system.

Once the desired residence time has elapsed, the gastric residence system passes out of the stomach. To do so, various components of the gastric residence system are designed to weaken and degrade. The specific dimensions of the system are also taken into consideration. In its intact, open configuration, the gastric residence system is designed to resist passage through the pylorus. However, some laser linker components of the gastric residence system are chosen such that they gradually degrade over the specified residence period in the stomach. When the laser linker components are sufficiently weakened by degradation, the gastric residence system loses critical resilience to compression or size reduction and can break apart into smaller pieces. The reduced-size system and any smaller pieces are designed to pass through the pylorus. The system then passes through the intestines and is eliminated from the patient. In some embodiments, a gastric residence system may be designed to weaken at specific locations such that the gastric residence system can pass through a pyloric valve intact once the residence time expires without degrading into numerous smaller pieces.

### Gastric Residence System Configuration

Gastric residence systems can be prepared in different configurations. A gastric residence system according to some examples of the disclosure includes one or more retention members. A retention member may be a component of a gastric residence system which helps the gastric residence system to avoid premature passage through the pyloric sphincter (e.g., by expanding or unfolding in a gastric environment). In some embodiments, a retention member includes at least one drug eluting component. A drug eluting component may include an agent (an active pharmaceutical ingredient, such as a therapeutic agent). The drug eluting component may further include one or more carrier polymers and/or other excipients or additives (e.g., stabilizers or dispersants).

The drug eluting component may be attached to at least one linker component, for example via laser welding, infrared welding, adhesion, or any other suitable method of attachment. Linker components may be referred to as “laser linker components” when the linker components are attached to drug eluting components using the laser welding techniques described herein.

In some embodiments, laser linker components may be inactive components, time dependent disintegrating matrix components, or enteric disintegrating matrix components. In some embodiments, a given retention member may comprise a plurality of laser linker components. For example, one or more laser linker components may be laser welded to the drug eluting component of a retention member. In another example, one or more laser linker components may be welded to one or more additional laser linker components within the retention member. In some embodiments, a retention member comprises multiple types of laser linker components. For example, a retention member may include one or more inactive components, one or more time dependent disintegrating matrix components, and/or one or more enteric disintegrating matrix components.

In some embodiments, a retention member may exclude a drug eluting component. A retention member without a drug eluting component may comprise one or more laser linker components.

In some embodiments, a gastric residence system may comprise multiple retention members. For example, a gastric residence system may include at least two retention members, at least three retention members, at least four retention members, at least five retention members, or at least six retention members. In some embodiments, one or more retention members may include a drug eluting component. For example, in a gastric residence system with six retention members, one retention member may include a drug eluting component while five retention members exclude a drug eluting component; two retention members may include a drug eluting component while four retention members exclude a drug eluting component; three retention members may include a drug eluting component while three retention members exclude a drug eluting component; four retention members may include a drug eluting component while two retention members exclude a drug eluting component; five retention members may include a drug eluting component while one retention member excludes a drug eluting component; or all six retention members may include a drug eluting component.

In some embodiments, the one or more retention members may be attached to an elastomeric component. The elastomeric component may enable the gastric residence system to be compacted, such as by being folded or compressed, into a form suitable for administration to the stomach by swallowing a container or capsule containing the compacted system. Upon dissolution of the capsule in the stomach, the gastric residence system expands into a shape which prevents passage of the system through the pyloric sphincter of the patient for the desired residence time of the system. Thus, the elastomeric component must be capable of being stored in a compacted configuration in a capsule for a reasonable shelf life, and of expanding to its original shape, or approximately its original shape, upon release from the capsule without breaking due to torque or stress.

In some embodiments, the elastomeric component may comprise an elastomer (also referred to as an elastic polymer or a tensile polymer) overmolded onto one or more intercomponent anchors. An intercomponent anchor serves to link, or anchor, different components of the gastric residence system (e.g., the elastomeric component and a retention member) together. In some embodiments, intercomponent anchors may comprise any suitable polymer that adheres or joins well to the components to be linked (e.g., polycarbonate, polyphenylsulfone, a polyphenylene ether-polystyrene blend, polyphenylene ether, polystyrene, or polyether ether ketone). In some embodiments, an intercomponent anchor may be “dumbbell” shaped, comprising relatively thicker lobes at either end of the anchor joined together by a thinner connector portion. Optionally, the intercomponent anchors can have a larger central body from which the first portion and second portion project in opposite directions from each other. The larger central body can have the dimensions of the retention members of the gastric residence system, so as to form an intermediate region between the central elastomer and the remainder of the retention members of the gastric residence system.

In some embodiments, an elastomer may be overmolded onto a first portion of an intercomponent anchor (e.g., onto one lobe of a dumbbell-shaped anchor). In some embodiments, it may be challenging to directly laser weld the elastomer to the one or more retention members. To facilitate the attachment of the retention members to the elastomeric component, a laser linker component may be overmolded onto a second portion of the intercomponent anchor (e.g., onto the second lobe of the dumbbell). In some embodiments, the laser linker component overmolded onto the intercomponent anchor may be an inactive component (e.g., polycaprolactone). A retention member may then be laser welded to the laser linker component to attach the retention member to the elastomeric component. In some embodiments, the elastomeric component may comprise a plurality of intercomponent anchors overmolded with elastomer, such that the elastomer holds one lobe of each of the plurality of intercomponent anchors together. A laser linker component may be overmolded onto the other lobe of each intercomponent anchor, such that multiple retention members may be laser welded to the elastomeric component via the laser linker components.

In some embodiments, the resulting gastric residence system configuration is a stellate configuration, also known as a “star” or “asterisk” configuration, wherein the elastomeric component is in the center of the system and the retention members are “arms” which extend radially outward from the elastomeric component.

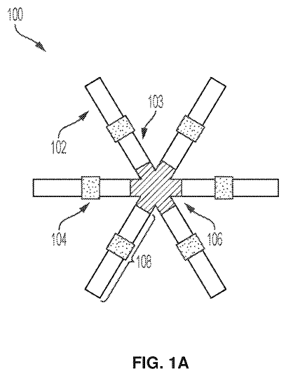

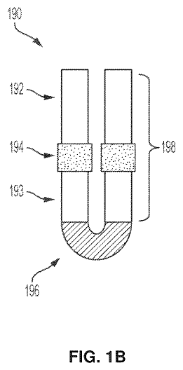

A simplified exemplary stellate system 100 is shown schematically in FIG. 1A. Multiple retention members (only one such retention member, 108, is labeled for clarity), are affixed to disk-shaped elastomeric component 106. The retention members depicted in FIG. 1A are comprised of segments 102 and 103, joined by a laser linker component 104 (again, the components are only labeled in one retention member for clarity). One or both of segments 102 and 103 may be a drug eluting component. Segments 102 and/or 103 may alternatively be additional laser linker components. While the laser linker components 104 are shown as slightly larger in diameter than the segments 102 and 103 in FIG. 1A, they can be the same diameter as the segments, so that the entire retention member 102-104-103 has a smooth outer surface. The stellate configuration depicted in FIG. 1A can be folded or compacted at the elastomeric component.

FIG. 1B shows a folded configuration 190 of the gastric residence system of FIG. 1A (for clarity, only two retention members are illustrated in FIG. 1B). Segments 192 and 193, laser linker component 194, elastomeric component 196, and retention member 198 of FIG. 1B correspond to segments 102 and 103, laser linker component 104, elastomeric component 106, and retention member 108 of FIG. 1A, respectively. When folded, the overall length of the gastric residence system is reduced by approximately a factor of two, and the system can be conveniently placed in a container such as a capsule or other container suitable for oral administration. The gastric residence system is constrained by the capsule or other container into the compacted state (the folded state). When the capsule reaches the stomach, the capsule dissolves, releasing the gastric residence system. Upon release of the constraint by the capsule or other container, the gastric residence system then unfolds into its uncompacted state, which is retained in the stomach for the desired residence period.

However, it has been demonstrated that gastric residence systems of a stellate shape can bend into a configuration that allows for premature passage through the pylorus of a patient. Gastric residence systems that prematurely pass through the pylorus fail to deliver the agent of the gastric residence system to the patient. Further, premature passage causes inconsistency, causes unreliability, and compromises the efficacy of the gastric residence system.

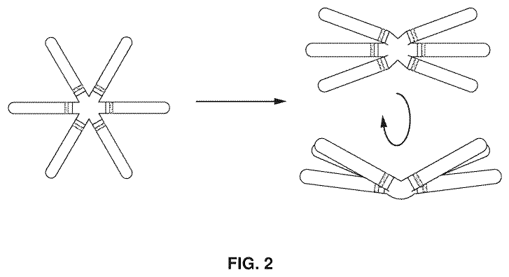

FIG. 2 shows a stellate-shaped gastric residence system having a plurality of retention members. One example of a bended configuration is shown on the right side of FIG. 2. Due to forces in the stomach (e.g., peristaltic forces), gastric residence systems may bend into configurations, such as that shown in FIG. 2, that can allow for premature passage through the pylorus.

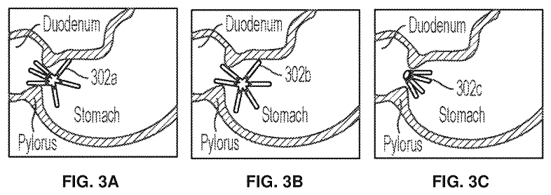

Other possible bended configurations are shown in FIGS. 3A-3C. Specifically, FIGS. 3A-3C show three different configurations that a gastric residence system may assume that can allow for premature passage through the pylorus. As shown in each figure, the relatively stiff retention members of the gastric residence system remain straight. However, because the elastomeric component of the gastric residence system has a higher flexibility than the retention members, the elastomeric component can bend. The bending of the elastomeric component can allow gastric residence systems having relatively stiff retention members to prematurely pass through the pylorus of a patient.

As shown in FIG. 3A, gastric residence system 302a is shown in a bended configuration having three retention members leading through the pyloric opening. FIG. 3B shows gastric residence system 302b in a bended configuration having two retention members leading through the pyloric opening. FIG. 3C shows gastric residence system 302c in a bended configuration analogous to the shape of a shuttlecock and having the elastomeric component leading through the pyloric opening.

To help prevent premature passage through the pylorus, gastric residence systems may be assembled using the laser welding techniques described herein. Laser welding components of a gastric residence system together may strengthen the interfaces between components, allowing them to resist breakage and premature passage through the pylorus.

### Laser Welding of Gastric Residence Systems

As described above, the gastric residence systems described herein may be assembled using laser welding techniques. For instance, individual components of a retention member (e.g., one or more drug eluting components and/or laser linker components) may be laser welded together to form a retention member. Laser welding may also be used to attach retention members to an elastomeric component to form a larger gastric residence system (e.g., a stellate system). Using laser welding to join components of a gastric residence system may offer several improvements over conventional techniques. For example, laser welding may create strong bonds between components even if the components have different compositions, properties, and/or functions, while conventional techniques may require components to comprise similar polymers in order to adhere effectively. Laser welding also does not introduce any additional components into a gastric residence system, which minimizes the number of failure points in a gastric residence system and avoids introducing components which are incompatible with human consumption. Furthermore, laser welding may be applied in a manner that prevents damage to heat-sensitive components, such as drug eluting components containing active pharmaceutical ingredients.

Various laser systems may be used for laser welding components of a gastric residence system. In some embodiments, a near infrared laser may be used. The wavelength of the laser may be about 1000-3000 nm, about 1500-2500 nm, about 1800-2000 nm, or about 1940 nm. In some embodiments, the wavelength of the laser used may be selected based on the absorbance of the polymers being welded. In some embodiments, the laser system may be a Dukane laser welder. In some embodiments, the laser source model may be an IPG TLM-120-WC-Y12 laser or a Sakar PE 2000 AC laser. In some embodiments, the laser scan head may be a SCANcube 10 or a Raylase 11532 scan head. In some embodiments, a single laser scan head may be used in order to weld a single gastric residence system. In some embodiments, multiple laser scan heads (e.g., dual laser scan heads) may be used in order to weld multiple gastric residence systems simultaneously.

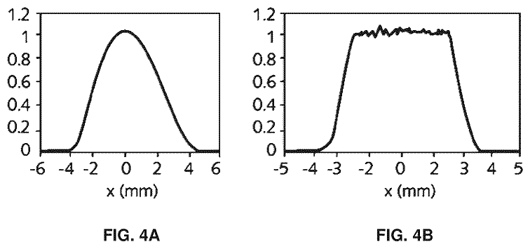

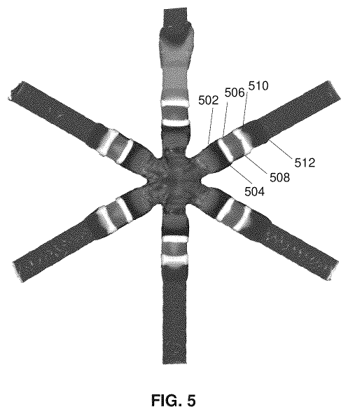

In some embodiments, the laser beam may have a Gaussian energy profile. For a beam with a Gaussian energy profile, the highest concentration of energy is in the center of the beam, and the concentration of energy decreases radially outward. An exemplary Gaussian energy profile is shown in FIG. 4A. In some embodiments, the laser beam may have a flat top or “top hat” energy profile. For a beam with a top hat energy profile, the concentration of the energy is flat and uniform across the width of the beam, with sharp edges where the energy concentration drops rapidly to zero. An exemplary top hat energy profile is shown in FIG. 4B. In some embodiments, other laser beam profiles may be used, such as a diffraction energy profile or a reverse-Gaussian energy profile.

In some embodiments, the laser beam may have a diameter or spot size of about 0.5-5 mm, about 0.75-4 mm, or about 1-3 mm. Different beam diameters may be used depending on the goals of the laser welding process. For example, using a larger beam diameter may be more efficient than using a smaller beam and may ensure the weld melts the entire interface. On the other hand, if the components to be welded together are very small, a smaller diameter laser beam may be more desirable because it may be easier to control and may enable welding of a single interface. However, if the beam diameter is too small, the beam may be too concentrated and burn through the components to be welded.

The power and energy of the laser may be varied based on the composition of the components being welded. For example, the power and energy used to laser weld a laser linker component to a drug eluting component may differ from the power and energy used to laser weld as laser linker component to another laser linker component. In some embodiments, the power of the laser may be about 10-1200 W, about 20-1000 W, or about 30-800 W. In some embodiments, the power of the laser may be at least about 10 W, at least about 20 W, or at least about 30 W. In some embodiments, the power of the laser may be less than about 1200 W, less than about 1000 W, or less than about 800 W. In some embodiments, the energy of the laser may be about 10-750 J, about 30-500 J, or about 40-450 J. In some embodiments, the energy of the laser may be at least about 10 J, at least about 30 J, or at least about 40 J. In some embodiments, the energy of the laser may be less than about 750 J, less than about 500 J, or less than about 450 J.

In some embodiments, the speed of the laser may be varied depending on the amount of energy desired to be applied to a particular interface. For example, if a certain interface requires more energy to weld, the power of the laser can be kept constant while decreasing the speed, thus increasing the amount of time the laser is incident on any given point. In some embodiments, the speed of the laser may be about 500-5000 mm/s, about 700-4500 mm/s, or about 900-4000 mm/s. In some embodiments, the speed of the laser may be at least about 500 mm/s, at least about 700 mm/s, at least about 1000 mm/s, at least about 2000 mm/s, at least about 3000 mm/s, at least about 3500 mm/s, at least about 4000 mm/s, or at least about 5000 mm/s. In some embodiments, the speed of the laser may be less than about 5000 mm/s, less than about 4000 mm/s, 3500 mm/s, less than about 3000 mm/s, less than about 2000 mm/s, less than about 1000 mm/s, less than about 700 mm/s, or less than about 500 mm/s.

In some embodiments, the characteristics of the laser beam may be selected in order to ensure that the beam effectively heats through the entire weld interface all the way to the bottom. In some embodiments, the beam is configured to heat through about 90-100% of the interface. In some embodiments, the beam is configured to heat through at least about 90% of the interface, at least about 95% of the interface, or at least about 99% of the interface. If only the top portion of the interface, which receives direct contact from the laser, is melted, the weld may be weak and/or incomplete, which may lead to breakage of the retention members of the gastric residence system. In some embodiments, this consideration may be balanced against the desire to avoid burning the top portion of the interface.

In some embodiments, a beam with a 3 mm diameter and a top hat energy profile may be used. The energy of the beam may be uniform across the 3 mm diameter, which may melt not only the interface between the components to be welded but also some or all of the components themselves. In some embodiments, the laser beam may produce a melt zone between laser welded components of about 0.5-5 mm, about 0.75-4 mm, or about 1-3 mm. In some embodiments, the melt zone between laser welded components may be at least about 0.5 mm, at least about 0.75 mm, or at least about 1 mm. In some embodiments, the melt zone between laser welded components may be less than about 5 mm, less than about 4 mm, or less than about 3 mm.

In some embodiments, melting through some or all of the components may be advantageous. For example, in some embodiments, the components to be welded together may each include a polyester, such as PCL. The polymer domains (e.g., PCL domains) of the components may be oriented along an axis (e.g., along an extrusion axis). Using PCL as an example, when the components are melted, the crystalline PCL melts, causing the PCL domains to be oriented randomly. If only a portion of the component is melted, that portion of the component may have randomly oriented PCL domains, well the un-melted portion of the component may have uniformly oriented PCL domains. The difference in orientation may cause breaking or bending where those portions meet. Thus, for certain components, it may be desirable to melt entire components during laser welding instead of just the interfaces between components. This may not be desirable for all components, however. For example, drug eluting components may not be melted entirely in order to avoid damaging the active pharmaceutical ingredient.

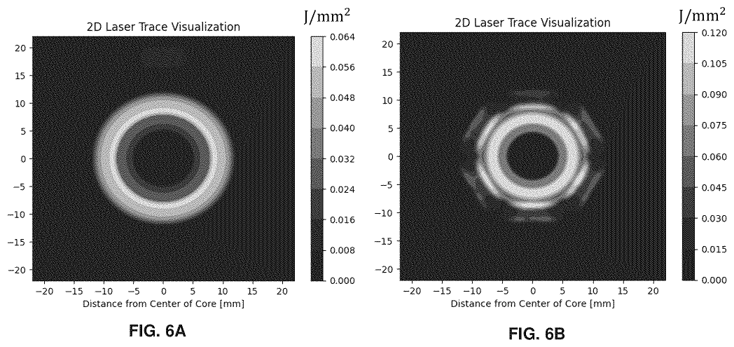

In some embodiments, if the gastric residence system includes multiple corresponding interfaces that need to be welded together (such as a stellate that includes a plurality of retention members), the laser beam may be moved along a repetitive path, such as a circular path. For example, for a stellate system, the laser beam may be moved along a circular path which is symmetrical about the center of the stellate system and may trace the same interface on each retention member of the stellate system, such that corresponding interfaces on the different retention members are heated as the laser beam moves along the circular path. A laser beam may be configured to follow multiple repetitive paths in order to laser weld a given system, wherein each repetitive path corresponds to a different set of interfaces to be welded. In some embodiments, the laser beam may be configured to follow at least one repetitive path, at least two repetitive paths, at least three repetitive paths, at least four repetitive paths, at least five repetitive paths, or at least six repetitive paths for a given stellate system. In some embodiments, repetitive paths may be welded in order moving radially outward from the center of the system (e.g., the first repetitive path may weld the interfaces closest to the elastomeric component, while subsequent repetitive paths weld the interfaces further from the elastomeric component). For example, in FIG. 5, which shows an exemplary laser welded stellate system, the circular path corresponding to interface 502 may be laser welded first, the circular path corresponding to interface 504 may be laser welded second, the circular path corresponding to interface 506 may be laser welded third, and so on. In other embodiments, repetitive paths may be welded in order moving radially inward from the outer edges of the system to the center of the system.

In some embodiments, the laser beam may make multiple passes around a given repetitive path in order to ensure a complete weld (i.e., to ensure that the laser beam melts the entire interface between the welded components) before moving on to another repetitive path. In some embodiments, the laser beam may make about 10-2000 passes around a given path, about 25-1500 passes around a given path, or about 40-1300 passes around a given path. In some embodiments, the laser beam may make at least about 10 passes around a given path, at least about 25 passes around a given path, or at least about 40 passes around a given path. In some embodiments, the laser beam may make less than about 2000 passes around a given path, less than about 1500 passes around a given path, or less than about 1300 passes around a given path.

In some embodiments, the laser beam may follow a given path for about 1-10 seconds, about 2-8 seconds, or about 3-6 seconds. In some embodiments, the laser beam may follow a given path for at least about 1 second, at least about 2 seconds, or at least about 3 seconds. In some embodiments, the laser beam may follow a given path for less than about 10 seconds, less than about 8 seconds, or less than about 6 seconds.

Once the laser beam finishes welding a given path, the gastric residence system may be allowed to cool in place before welding a subsequent path. In some embodiments, the gastric residence system may be allowed to cool for about 1-20 seconds, about 5-15 seconds, or about 8-10 seconds before starting to weld the next path. In some embodiments, the gastric residence system may be allowed to cool for at least about 1 second, at least about 5 seconds, or at least about 8 seconds before starting to weld the next path. In some embodiments, the gastric residence system may be allowed to cool for less than about 20 seconds, less than about 15 seconds, or less than about 10 seconds before starting to weld the next path.

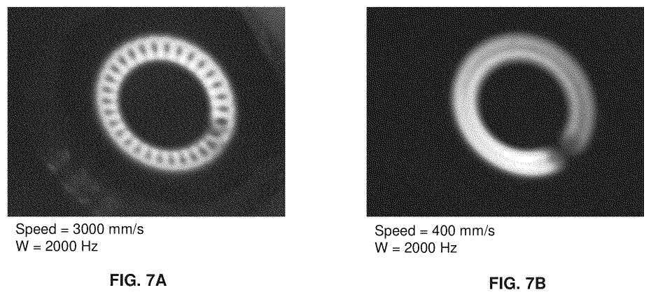

In some embodiments, the settings of the laser may be varied for different paths. For example, the energy and/or power of the laser required to laser weld a drug eluting component to a laser linker component may differ from the energy and/or power required to laser weld two laser linker components. FIG. 6A shows an exemplary heat map of the energies provided to the various portions of a stellate system. As shown, the energies of the paths following different sets of interfaces of the stellate vary based on the energy needs of the interfaces being laser welded.

In some embodiments, one or more interfaces of the gastric residence system may be welded individually instead of following a repetitive path around the gastric residence system. For example, some retention members of the gastric residence system may include drug eluting components, while other retention members do not. As such, interfaces on drug eluting retention members may not have corresponding interfaces on the other retention members. Thus, certain interfaces on the drug eluting retention members may be welded separately. Examples of separately welded interfaces are shown at the top of FIG. 6A and in FIG. 6B. In some embodiments, separately welded interfaces are welded by moving the laser beam back and forth across a single interface before moving on to another interface. This type of weld may be referred to herein as a “hex weld.” In some embodiments, the laser beam may be passed across a single interface in a hex weld about 10-1000 times, about 25-500 times, or about 50-400 times. In some embodiments, the laser beam may be passed across a single interface in a hex weld at least about 10 times, at least about 25 times, or at least about 50 times. In some embodiments, the laser beam may be passed across a single interface in a hex weld less than about 1000 times, less than about 500 times, or less than about 400 times.

In some embodiments, to avoid burning the components to be welded, the laser beam may be “wobbled” about the interface(s) to be welded. When a beam is wobbled, the beam does not exactly follow the seam between the components to be welded but rather moves in a regular or irregular pattern around the seam. Thus, wobbling a beam may effectively increase the diameter of the beam. In some embodiments, the wobble radius of a wobbled beam may be about 0.5-10 mm, about 0.7-5 mm, or about 1-3 mm. In some embodiments, the wobble radius of a wobbled beam may be at least about 0.5 mm, at least about 0.7 mm, or at least about 1 mm. In some embodiments, the wobble radius of a wobbled beam may be less than about 10 mm, less than about 5 mm, or less than about 3 mm. Wobbling may be used for beams having any energy profile, including a Gaussian energy profile or a top hat energy profile.

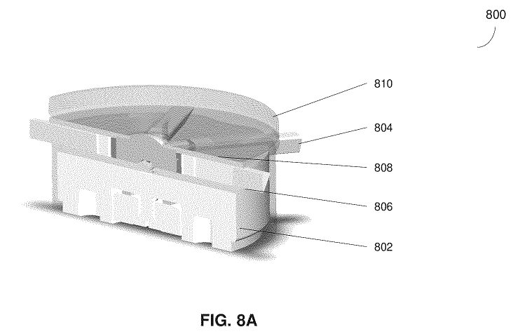

In some embodiments, wobbling a beam may create “hot spots” and “cold spots” in the beam's energy profile. For example, “hot spots” may be created if the wobble pattern causes the laser beam to hit the same location repeatedly. Similarly, “cold spots” may be created if the wobble pattern causes the laser beam to hit certain locations less frequently or skip them altogether (e.g., if a helical wobble pattern is used). FIGS. 7A and 7B show exemplary circular paths with a wobble. As shown in FIG. 7A, wobbling the beam in a helical pattern creates “cold spots” in the middle of the helix. The incidence of hot spots and/or cold spots may be controlled by changing the speed of the laser or the frequency of the wobble. For example, if the speed of the laser and/or the frequency of the wobble is decreased, (e.g., from 3000 mm/s as shown in FIG. 7A to 400 mm/s as shown in FIG. 7B), the hot spots and/or cold spots may blur together, creating a more evenly distributed energy profile. In some embodiments, the frequency of the wobble may be about 500-5000 Hz, about 1000-4000 Hz, or about 1200-3200 Hz. In some embodiments, the frequency of the wobble may be at least about 500 Hz, at least about 1000 Hz, or at least about 1200 Hz. In some embodiments, the frequency of the wobble may be less than about 5000 Hz, less than about 4000 Hz, or less than about 3200 Hz.

In some embodiments, wobbling may be used to create hot spots and/or cold spots intentionally. For example, if two materials to be welded melt at different temperatures, it may be desirable to employ a wobble pattern which imparts more energy to the material which melts at a higher temperature and less energy to the material which melts at a lower temperature.

In some embodiments, the relative humidity of the environment in which laser welding is performed may be controlled. Weld strength may be weakened if the components being laser welded absorb water before laser welding. Accordingly, it may be desirable to control the relatively humidity of the laser welding environment, although laser welding may still be effective at relative humidities higher than those contemplated herein. In some embodiments, it may be desirable to maintain at least some humidity in the room because the components to be laser welded may become brittle and/or may experience an increase in static cling if the humidity is too low. In some embodiments, the relative humidity during laser welding may be about 10-40%, about 12-35%, or about 15-25%. In some embodiments, the relative humidity during laser welding may be less than about 40%, less than about 35%, less than about 30%, less than about 25%, less than about 20%, or less than about 15%. In some embodiments, the relative humidity during laser welding may be at least about 10%, at least about 15%, at least about 20%, at least about 25%, at least about 30%, or at least about 35%. In some embodiments, the components to be laser welded are exposed to the relative humidity in the environment in which the laser welding is performed for less than 1 hour, less than 45 minutes, less than 30 minutes, less than 25 minutes, or less than 20 minutes.

In some embodiments, once all interfaces of a gastric residence system have been welded, the gastric residence system may be allowed to cool. The gastric residence system may be allowed to cool in place (e.g., in the laser welding holder in which the system was welded) or may be moved (e.g., removed from the laser welding holder) before cooling. In some embodiments, the gastric residence system may be passively cooled using ambient air. In some embodiments, the gastric residence system may be actively cooled (e.g., by an air cooling system). In some embodiments, the gastric residence system may be allowed to cool for about 10 seconds-5 minutes, about 30 seconds-4 minutes, about 1-3 minutes, or about 2 minutes.

### Laser Welding Assembly

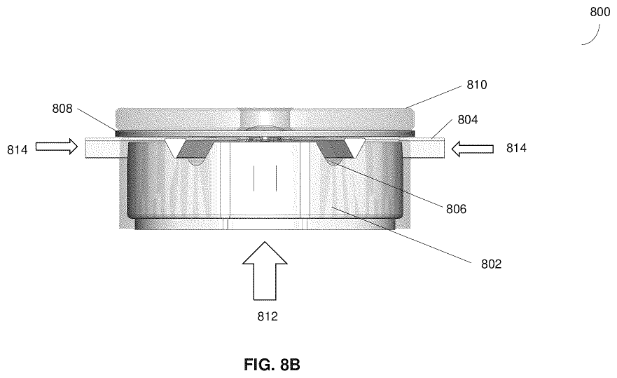

A laser welding assembly may be used to hold the components of the system to be welded (such as a gastric residence system) in place during laser welding. An exemplary laser welding assembly for a stellate-shaped gastric residence system is shown in FIGS. 8A and 8B. The laser welding assembly 800 may include a laser welding holder 802 comprising grooves sized and shaped to receive the components of a gastric residence system 804 (e.g., a stellate). The grooves of laser welding holder 802 may be covered with a lining 806. The laser welding assembly 800 may further include a top layer 808 configured to hold the components of gastric residence system 804 in place during laser welding and a separate glass layer 810 which sits on top of top layer 808. The laser welding assembly 800 may also include a vertical pressure clamp 812 and one or more radial pressure clamps 814, which may be configured to apply vertical and radial forces, respectively, during laser welding.

A laser welding assembly 800 includes a laser welding holder 802 with grooves sized and shaped to receive the components of a gastric residence system. Laser welding holder 802 may be circular (e.g., like a puck), polygon-shaped (e.g., triangular, square, or rectangular), irregularly shaped, or any other suitable shape. Laser welding holder 802 may help maintain part geometry during laser welding by preventing the various components of gastric residence system 804 from flowing when in a molten state. Laser welding holder 802 may be formed from metal, such as stainless steel (e.g., 3000 series stainless steel) or aluminum (e.g., 5000 series aluminum or 6000 series aluminum). In some embodiments, laser welding holder 802 may be coated, for example by electroless nickel plating.

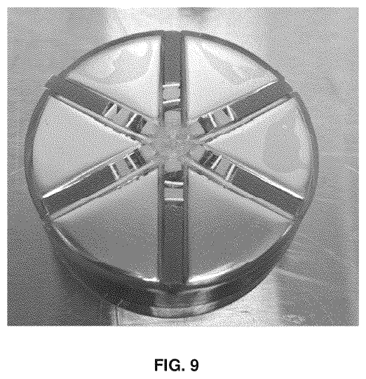

In some embodiments, laser welding holder 802 is a circular shape, or puck, as shown in FIG. 9. In some embodiments, laser welding holder 802 may be at least about 11 mm thick, at least about 12 mm thick, at least about 13 mm thick, at least about 14 mm thick, at least about 15 mm thick, at least about 16 mm thick, at least about 17 mm thick, at least about 18 mm thick, or at least about 19 mm thick. In some embodiments, laser welding holder 802 may be less than about 20 mm thick, less than about 19 mm thick, less than about 18 mm thick, less than about 17 mm thick, less than about 16 mm thick, less than about 15 mm thick, less than about 14 mm thick, less than about 13 mm thick, or less than about 12 mm thick. In some embodiments, the diameter of laser welding holder 802 may be at least about 35 mm, at least about 40 mm, at least about 45 mm, or at least about 50 mm. In some embodiments, the diameter of the laser welding holder may be less than about 52 mm, less than about 47 mm, less than about 42 mm, or less than about 37 mm.

In some embodiments, the upper surface of laser welding holder 802, including the grooves, may be covered with a lining 806. Lining 806 may be a nonstick layer and may comprise silicone (e.g., a liquid silicone rubber such as Dow Corning QP1-250 liquid silicone rubber, 50A durometer), polytetrafluoroethylene, ceramics, or any other suitable nonstick material. Lining 806, in combination with laser welding holder 802, may prevent gastric residence system 804 from sticking to laser welding holder 802 during removal. Lining 806 may also help to prevent the flow of molten components of gastric residence system 804 during laser welding. Furthermore, lining 806 may mitigate flashing during welding. In some embodiments, lining 806 may be at least about 0.75 mm thick, at least about 0.80 mm thick, at least about 0.85 mm thick, at least about 0.90 mm thick, or at least about 0.95 mm thick. In some embodiments, lining 806 may be less than about 1 mm thick, less than about 0.95 mm thick, less than about 0.90 mm thick, less than about 0.85 mm thick, or less than about 0.80 mm thick.

In some embodiments, laser welding assembly 800 may further include a top layer 808 configured to hold gastric residence system 804 in place within the grooves of laser welding holder 802. In some embodiments, top layer 808 may comprise a non-stick material in order to prevent gastric residence system 804 from sticking to top layer 808 once laser welding is complete. Top layer 808 may comprise silicone (e.g., Specialty Silicone Products SSP-2390-40 silicone sheet, 40A durometer), glass, polytetrafluoroethylene, ceramics, or any other suitable non-stick material. The length and width of top layer 808 may match the length and width of the top of laser welding holder 802. For example, if laser welding holder 802 has a circular cross-section, the diameter of top layer 808 may match the diameter of laser welding holder 802. In some embodiments, top layer 808 may be at least about 0.025 in thick, at least about 0.027 in thick, at least about 0.029 in thick, at least about 0.031 in thick, or at least about 0.033 in thick. In some embodiments, top layer 808 may be less than about 0.035 in thick, less than about 0.033 in thick, less than about 0.031 in thick, less than about 0.029 in thick, or less than about 0.027 in thick.

In some embodiments, laser welding assembly 800 may further include a glass layer 810 which sits on top of top layer 808. Glass layer 810 may be formed from quartz (e.g., Momentive quartz 124). In some embodiments, glass layer 810 may be at least about 0.05 in thick, at least about 0.10 in thick, or at least about 0.15 in thick. In some embodiments, glass layer 810 may be less than about 0.20 in thick, less than about 0.15 in thick, or less than about 0.10 in thick.

In some embodiments, laser welding assembly 800 may include one or more vertical pressure clamps 812 configured to apply a force to one or more portions of laser welding assembly 800 during laser welding to prevent the flow of molten gastric residence system components. Vertical pressure clamp 812 may apply a downward force. The downward force applied during welding may be about 100-5000 N, about 150-3000 N, or about 200-2800 N. In some embodiments, the downward force applied during welding may be at least about 100 N, at least about 200 N, or at least about 250 N. In some embodiments, the downward force applied during welding may be less than about 5000 N, less than about 4000 N, less than about 3000 N, or less than about 2800 N.

In some embodiments, laser welding assembly 800 may include one or more radial pressure clamps 814 configured to apply a radial force to laser welding assembly 800. In some embodiments, a radial force may be applied to each retention member of a stellate-shaped gastric residence system 804 using pistons positioned at the end of each groove in laser welding holder 802. In some embodiments, the radial force applied by each piston may be about 5-200 N, about 8-100 N, or about 10-50 N. In some embodiments, the radial force applied by each piston may be at least about 5 N, at least about 8 N, or at least about 10 N. In some embodiments, the radial force applied by each piston may be less than about 200 N, less than about 100 N, or less than about 50 N.

The pistons used to apply radial force may be wider than the retention members or may be stilettos which are narrower than the retention members. In some embodiments, the width of the pistons may be selected based on whether the retention members of gastric residence system 804 extend past the edge of laser welding holder 802. For example, pistons wider than the retention members may be used if the retention members are shorter than the radius of laser welding holder 802, and pistons narrower than the retention members may be used if the retention members are longer than the radius of laser welding holder 802 and extend past the edge of laser welding holder 802.

In some embodiments, the radial force and downward force may be applied in a stepwise manner in order to prevent gastric residence system 804 from popping out of the grooves of laser welding holder 802 or becoming deformed prior to welding. For example, a relatively low downward force (e.g., about 20 PSI) may be applied by vertical pressure clamp 812. A relatively high radial force (e.g., about 65 PSI) may then be applied by the radial pistons of radial pressure clamps 814. Subsequently, a larger downward force (e.g., about 80 PSI) may be applied by vertical pressure clamp 812. The radial force may then be decreased (e.g., to about 15 PSI), at which point the welding process may begin.

### Selection of Materials for Laser Welding

Certain polymers may be challenging to laser weld together, which may result in poor weld strength (and, as a result, poor gastric retention). Accordingly, the compositions and/or properties of the various components of a laser welded system (e.g., a gastric residence system) may be selected to facilitate laser welding of the components.

In some embodiments, each component of a retention member of a gastric residence system (e.g., each laser linker component and/or drug eluting component) may comprise a polymer in common. A “common polymer” may refer to a polymer which is present in an identical form in each component (e.g., a polymer which has the same molecular weight in each component) or may be a polymer which has the same empirical formula but a different molecular formula in the various components (e.g., a polymer which has a different molecular weight in different components). The common polymer present in each component may include a polyester (e.g., polycaprolactone (PCL)), a thermoplastic co-polyester, a thermoplastic polyurethane, a thermoplastic polyamide, a thermoplastic vulcanizate, a styrenic block copolymer, polyethylene co-vinyl acetate, or polylactic acid. Including a common polymer in each component of a retention member may improve the strength of the welds between components because similar polymers may be easier to laser weld together. Polymers may be considered “similar” if, for example, they are of the same type or class (e.g., thermoplastics, thermosets, elastomers, etc.), have similar melting temperatures (e.g., have melting temperatures within about 100° C. of one another), and/or have similar viscosities at their melting temperatures and pressures (e.g., have viscosities within about 50% of one another). Thus, including a common polymer in each component of a gastric residence system to improve weld strength may improve gastric retention by preventing components of the gastric residence system from breaking and causing the system to pass through the pylorus prematurely.

In some embodiments, PCL may be a common polymer present in each component of the retention members of a gastric residence system. In some embodiments, PCL may be present as PCL12, PCL17, or any other suitable form of PCL. In some embodiments, each component may comprise at least about 30% by weight PCL, at least about 35% by weight PCL, at least about 40% by weight PCL, at least about 45% by weight PCL, or at least about 50% by weight PCL. In some embodiments, each component may comprise less than about 75% by weight PCL, less than about 65% by weight PCL, or less than about 50% by weight PCL.

In some embodiments, neighboring components (i.e., components to be laser welded together) may have similar viscosities or melt flow indices. Laser welding polymers with similar viscosities or melt flow indices may cause less phase separation than laser welding polymers with vastly different viscosities or melt flow indices, which may lead to improved weld strength. Conventionally, polymers may be difficult to laser weld to one another unless the polymers have very close melt flow indices (e.g., melt flow indices within 10% of one another). However, by using the laser welding techniques described herein, components comprising polymers with greater differences in melt flow index may be laser welded together. In some embodiments, the difference between the melt flow indices of components to be laser welded may be at least about 10%, at least about 15%, at least about 20%, at least about 25%, or at least about 30%. In some embodiments, the difference between the melt flow indices of components to be laser welded may be less than about 50%, less than about 45%, less than about 40%, or less than about 35%.

In some embodiments, neighboring components may also have relatively similar melting temperatures. In some embodiments, neighboring components to be laser welded together may have melting temperatures within about 75° C. of one another, within about 60° C. of one another, within about 50° C. of one another, within about 40° C. of one another, within about 30° C. of one another, within about 20° C. of one another, within about 10° C. of one another, or within about 5° C. of one another.

In some embodiments, the composition of components of a retention member may be changed in order to bring viscosities, melt flow indices, and/or melting temperatures of neighboring components closer to one another to facilitate laser welding. For example, one or more excipients may be added to a laser linker component or drug eluting component in order to change (e.g., increase) the amount of laser energy the component can absorb. Excipients may include one or more of iron oxide, a colorant, or any other suitable absorption-enhancing material. In another example, a plasticizer (e.g., a poloxamer, such as P407) may be added to the thicker component of two components to be laser welded together in order to decrease the viscosity of the thicker component and bring the viscosities of the components closer together.

### Drug Eluting Component Composition—Agents

As described above, gastric residence systems may include one or more retention members with at least one drug eluting component. The drug eluting component of a retention member includes an agent (an active pharmaceutical ingredient, such as a therapeutic agent). In some embodiments, the agent in the drug eluting component is a drug, pro-drug, biologic, or any other substance which can be administered to produce a beneficial effect on an illness or injury. Exemplary agents that can be used in the gastric residence systems described herein include, but are not limited to, statins, such as rosuvastatin; nonsteroidal anti-inflammatory drugs (NSAIDs) such as meloxicam; selective serotonin reuptake inhibitors (SSRIs) such as escitalopram and citalopram; blood thinners, such as clopidogrel; steroids, such as prednisone; antipsychotics, such as aripiprazole and risperidone; analgesics, such as buprenorphine; opioid antagonists, such as naloxone; anti-asthmatics such as montelukast; anti-dementia drugs, such as memantine; cardiac glycosides such as digoxin; alpha blockers such as tamsulosin; cholesterol absorption inhibitors such as ezetimibe; anti-gout treatments, such as colchicine; antihistamines, such as loratadine and cetirizine, opioids, such as loperamide; proton-pump inhibitors, such as omeprazole; antiviral agents, such as entecavir; antibiotics, such as doxycycline, ciprofloxacin, and azithromycin; antimalarial agents; levothyroxine; substance abuse treatments, such as methadone and varenicline; contraceptives; stimulants, such as caffeine; and nutrients such as folic acid, calcium, iodine, iron, zinc, thiamine, niacin, vitamin C, vitamin D, biotin, plant extracts, phytohormones, and other vitamins or minerals. Biologics that can be used as agents in the gastric residence systems of the invention include proteins, polypeptides, polynucleotides, and hormones. Exemplary classes of agents include, but are not limited to, analgesics; anti-analgesics; anti-inflammatory drugs; antipyretics; antidepressants; antiepileptics; antipsychotic agents; neuroprotective agents; anti-proliferatives, such as anti-cancer agents; antihistamines; antimigraine drugs; hormones; prostaglandins; antimicrobials, such as antibiotics, antifungals, antivirals, and antiparasitics; anti-muscarinics; anxiolytics; bacteriostatics; immunosuppressant agents; sedatives; hypnotics; antipsychotics; bronchodilators; anti-asthma drugs; cardiovascular drugs; anesthetics; anti-coagulants; enzyme inhibitors; steroidal agents; steroidal or non-steroidal anti-inflammatory agents; corticosteroids; dopaminergics; electrolytes; gastro-intestinal drugs; muscle relaxants; nutritional agents; vitamins; parasympathomimetics; stimulants; anorectics; anti-narcoleptics; and antimalarial drugs, such as quinine, lumefantrine, chloroquine, amodiaquine, pyrimethamine, proguanil, chlorproguanil-dapsone, sulfonamides (such as sulfadoxine and sulfamethoxypyridazine), mefloquine, atovaquone, primaquine, halofantrine, doxycycline, clindamycin, artemisinin, and artemisinin derivatives (such as artemether, dihydroartemisinin, arteether and artesunate). The term “agent” includes salts, solvates, polymorphs, and co-crystals of the aforementioned substances. In some embodiments, the agent is selected from the group consisting of cetirizine, rosuvastatin, escitalopram, citalopram, risperidone, olanzapine, donepezil, and ivermectin. In some embodiments, the agent is one that is used to treat a neuropsychiatric disorder, such as an anti-psychotic agent such as risperidone.

Agents can be used in any suitable crystalline form, or in amorphous form, or in both crystalline form or forms and amorphous forms. That is, agent or drug particles contained in the drug eluting component of a retention member can be used in crystalline form, in amorphous form, or in a mixture of crystalline forms (either a single crystalline form, or multiple crystalline forms) and amorphous forms, so as to provide a desired rate of release or desired physical or chemical properties.

Gastric residence systems are well-suited for use in treatment of diseases and disorders which present difficulties with patient compliance, and thus in some embodiments, the gastric residence systems described herein are used to treat a disease or disorder where patient compliance with a medication regimen is problematic. Such diseases and disorders include neuropsychiatric diseases and disorders, dementia and other diseases and disorders which affect memory, Alzheimer's disease, psychoses, schizophrenia, and paranoia. Accordingly, agents which can be used in the drug eluting component of a retention member include, but are not limited to, anti-dementia agents, anti-Alzheimer's disease agents, and anti-psychotics.

Exemplary hydrophilic agents which can be used include risperidone, cetirizine, memantine, and olanzapine. Exemplary hydrophobic agents which can be used include aripiprazole, ivermectin, rosuvastatin, citalopram, and escitalopram.

In some embodiments, the agent can be loaded into a retention member or into a segment of a retention member in an amount ranging from about 10% to about 80% by weight of the retention member or of the segment of the retention member. Agent/API loading can be about 10% to about 70%, about 10% to about 60%, about 10% to about 50%, about 10% to about 40%, about 10% to about 30%, about 10% to about 20%, about 20% to about 80%, about 30% to about 80%, about 40% to about 80%, about 50% to about 80%, about 60% to about 80%, about 70% to about 80%, about 30% to about 70%, or about 40% to about 60% by weight of the retention member or of the segment of the retention member.

In some embodiments, the agent makes up about 10% to about 40% by weight of the retention member or segment of the retention member, and thus the carrier polymer and any other components of the retention member or segment of the retention member blended into the carrier polymer together make up the remainder of the weight of the retention member or segment of the retention member. In some embodiments, the agent or salt thereof makes up about 10% to about 35%, about 10% to about 30%, about 10% to about 25%, about 10% to about 20%, about 10% to about 15%, about 15% to about 40%, about 20% to about 40%, about 25% to about 40%, about 30% to about 40%, about 35% to about 40%, about 15% to about 35%, about 20% to about 35%, or about 25% to about 40% by weight of the retention member or segment of the retention member.

In some embodiments, the retention members, or segments of which the retention members are comprised, can have a high loading of agent. “High loading” generally refers to retention members or retention member segments where the agent makes up between about 40% by weight of the retention member or segment, to about 80% by weight of the retention member or segment. Any components of the retention members or retention member segments which are not blended into the carrier polymer are not included in the calculation of the weight percentage; for example, if a retention member has one or more disintegrating matrices interspersed between segments of the retention member, the weight of such matrices would not be included as part of the weight of the retention member in the calculation of the weight percentage of agent in the retention member.

In some embodiments, the amount of agent by weight in the retention members, or segments of which the retention members are comprised, can comprise at least about 40%, at least about 45%, at least about 50%, at least about 55%, or about 60%. In some embodiments, the amount of agent by weight in the retention members, or segments of which the retention members are comprised, can comprise about 40% to about 60%, about 45% to about 60%, about 50% to about 60%, about 55% to about 60%, about 40% to about 55%, about 40% to about 50%, or about 40% to about 45%. In some embodiments, the amount of agent by weight in the retention members, or segments of which the retention members are comprised, can comprise about 25% to about 60%, about 30% to about 60%, or about 35% to about 60%. In some embodiments, the amount of agent by weight in the retention members, or segments of which the retention members are comprised, can comprise about 51% to about 60%, about 52% to about 60%, about 53% to about 60%, about 54% to about 60%, about 55% to about 60%, about 56% to about 60%, or about 57% to about 60%.

The combination of the high agent loading with the release rate-controlling polymer film provides gastric residence systems with increased amounts of agent, while maintaining good release kinetics over the residence period of the system.

### Drug Eluting Component Composition—Carrier Polymers

In some embodiments, the agent in the drug eluting component may be blended with a carrier polymer. The resulting mixture can be formed into the desired shape or shapes for use as drug eluting components in the gastric residence systems described herein. After the agent is blended into the carrier polymer to form the carrier polymer-agent mixture, the agent is distributed or dispersed throughout the blended mixture. If excipients, anti-oxidants, or other ingredients are included in the carrier polymer-agent blend, they will also be distributed or dispersed throughout the blended mixture.

Selection of the carrier material for the agent in a gastric residence system influences the release profile of drug during the period of gastric residence. Carrier polymers may be thermoplastic, to allow extrusion using hot melt extrusion or 3D printing techniques. They may also have a high enough melt strength and viscosity to enable extrusion into the required geometry. They may have low melting temperatures (for example, less than about 120° C.), to avoid exposing agents to high temperatures during manufacture. They may have a sufficient mechanical strength (Young's modulus, compression strength, tensile strength) to avoid breaking in the stomach during the desired residence period. Further, they should be capable of forming stable blends with agents, therapeutic agents, drugs, excipients, dispersants, and other additives.

Exemplary carrier polymers suitable for use in the gastric residence systems described herein include, but are not limited to, hydrophilic cellulose derivatives (such as hydroxypropylmethyl cellulose, hydroxypropyl cellulose, hydroxymethyl cellulose, hydroxyethyl cellulose, carboxymethylcellulose, sodium-carboxymethylcellulose), cellulose acetate phthalate, poly(vinyl pyrrolidone), ethylene/vinyl alcohol copolymer, poly(vinyl alcohol), carboxyvinyl polymer (Carbomer), Carbopol® acidic carboxy polymer, polycarbophil, poly(ethyleneoxide) (Polyox WSR), polysaccharides and their derivatives, polyalkylene oxides, polyethylene glycols, chitosan, alginates, pectins, acacia, tragacanth, guar gum, locust bean gum, vinylpyrrolidonevinyl acetate copolymer, dextrans, natural gum, agar, agarose, sodium alginate, carrageenan, fucoidan, furcellaran, laminaran, hypnea, eucheuma, gum arabic, gum ghatti, gum karaya, arbinoglactan, amylopectin, gelatin, gellan, hyaluronic acid, pullulan, scleroglucan, xanthan, xyloglucan, maleic anhydride copolymers, ethylenemaleic anhydride copolymer, poly(hydroxyethyl methacrylate), ammoniomethacrylate copolymers (such as Eudragit RL or Eudragit RS), poly(ethylacrylate-methylmethacrylate) (Eudragit NE), Eudragit E (cationic copolymer based on dimethylamino ethyl methylacrylate and neutral methylacrylic acid esters), poly(acrylic acid), polymethacrylates/polyethacrylates such as poly(methacrylic acid), methylmethacrylates, and ethyl acrylates, polylactones such as poly(caprolactone), polyanhydrides such as poly [bis-(p-carboxyphenoxy)-propane anhydride], poly(terephthalic acid anhydride), polypeptides such as polylysine, polyglutamic acid, poly(ortho esters) such as copolymers of DETOSU with diols such as hexane diol, decane diol, cyclohexanedimethanol, ethylene glycol, polyethylene glycol and incorporated herein by reference those poly(ortho) esters described and disclosed in U.S. Pat. No. 4,304,767, starch, in particular pregelatinized starch, and starch-based polymers, carbomer, maltodextrins, amylomaltodextrins, dextrans, poly(2-ethyl-2-oxazoline), poly(ethyleneimine), polyurethane, poly(lactic acid), poly(glycolic acid), poly(lactic-co-glycolic acid) (PLGA), polyhydroxyalkanoates, polyhydroxybutyrate, poly(ethylene-co-vinyl acetate), and copolymers, mixtures, blends and combinations thereof. Polycaprolactone (PCL) and/or thermoplastic polyurethanes are preferred carrier polymers. In some embodiments, polydioxanone is used as the carrier polymer. In any of the embodiments of the gastric residence system, the carrier polymer used in the gastric residence system can comprise polycaprolactone, such as linear polycaprolactone with a number-average molecular weight (Mn) range between about 60 kiloDalton (kDa) to about 100 kDa; 75 kDa to 85 kDa; or about 80 kDa; or between about 45 kDa to about 55 kDa; or between about 50 kDa to about 110,000 kDa, or between about 80 kDa to about 110,000 kDa.

### Drug Eluting Component Composition—Excipients and Other Additives

Further, release of agent from the drug eluting component can be modulated by a wide variety of excipients included in the drug eluting component. Soluble excipients include P407, Eudragit E, PEG, Polyvinylpyrrolidone (PVP), and Polyvinyl alcohol (PVA). Insoluble, wicking excipients include Eudragit RS and Eudragit RL. Degradable excipients include PLA, PLGA, PLA-PCL, polydioxanone, and linear copolymers of caprolactone and glycolide; polyaxial block copolymers of glycolide, caprolactone, and trimethylene carbonate; polyaxial block copolymers of glycolide, trimethylene carbonate, and lactide; polyaxial block copolymers of glycolide, trimethylene carbonate and polypropylene succinate; polyaxial block copolymers of caprolactone, lactide, glycolide, and trimethylene carbonate; polyaxial block copolymers of glycolide, trimethylene carbonate, and caprolactone; and linear block copolymers of lactide, caprolactone, and trimethylene carbonate; such as linear copolymers of caprolactone (95%) and glycolide (5%); polyaxial block copolymers of glycolide (68%), caprolactone (29%), and trimethylene carbonate (3%); polyaxial block copolymers of glycolide (86%), trimethylene carbonate (9%), and lactide (5%); polyaxial block copolymers of glycolide (70%), trimethylene carbonate (27%) and polypropylene succinate (2%); polyaxial block copolymers of caprolactone (35%), lactide (34%), glycolide (17%), and trimethylene carbonate (14%); polyaxial block copolymers of glycolide (55%), trimethylene carbonate (25%), and caprolactone (20%); and linear block copolymers of lactide (39%), caprolactone (33%), and trimethylene carbonate (28%). Insoluble, swellable excipients include Polyvinyl acetate (PVAc), Crospovidone, Croscarmellose, HPMCAS, and linear block copolymers of dioxanone and ethylene glycol; linear block copolymers of lactide and ethylene glycol; linear block copolymers of lactide, ethylene glycol, trimethyl carbonate, and caprolactone; linear block copolymers of lactide, glycolide, and ethylene glycol; linear block copolymers of glycolide, polyethylene glycol, and ethylene glycol; such as linear block copolymers of dioxanone (80%) and ethylene glycol (20%); linear block copolymers of lactide (60%) and ethylene glycol (40%); linear block copolymers of lactide (68%), ethylene glycol (20%), trimethyl carbonate (10%), and caprolactone (2%); linear block copolymers of lactide (88%), glycolide (8%), and ethylene glycol (4%); linear block copolymers of glycolide (67%), polyethylene glycol (28%), and ethylene glycol (5%). Surfactants include Lecithin, Taurocholate, SDS, Soluplus, Fatty acids, and Kolliphor RH40.

Other excipients can be added to the carrier polymers to modulate the release of agent. Such excipients can be added in amounts from about 1% to 75%, from about 5% to 50%, or from about 5% or about 30%. Examples of such excipients include Poloxamer 407 (available as Kolliphor P407, Sigma Cat #62035), poly(ethylene glycol)-block-poly (propylene glycol)-block-poly(ethylene glycol), CAS No. 9003-11-6; H—(OCH2CH2)x-(O—CH(CH3)CH2)y-(OCH2CH2) z-OH where x and z are about 101 and y is about 56); Pluronic P407; Eudragit E, Eudragit EPO (available from Evonik); hypromellose (available from Sigma, Cat #H3785), Kolliphor RH40 (available from Sigma, Cat #07076), polyvinyl caprolactam, polyvinyl acetate (PVAc), polyvinylpyrrolidone (PVP), polyvinyl alcohol (PVA), polyethylene glycol (PEG), and Soluplus (available from BASF; a copolymer of polyvinyl caprolactam, polyvinyl acetate, and polyethylene glycol). Preferred soluble excipients include Eudragit E, polyethylene glycol (PEG), polyvinylpyrrolidone (PVP), polyvinyl acetate (PVAc), and polyvinyl alcohol (PVA). Preferred insoluble excipients include Eudragit RS and Eudragit RL. Preferred insoluble, swellable excipients include crospovidone, croscarmellose, hypromellose acetate succinate (HPMCAS), and carbopol. EUDRAGIT RS and EUDRAGIT RL are registered trademarks of Evonik (Darmstadt, Germany) for copolymers of ethyl acrylate, methyl methacrylate and methacrylic acid ester with quaternary ammonium groups (trimethylammonioethyl methacrylate chloride), having a molar ratio of ethyl acrylate, methyl methacrylate and trimethylammonioethyl methacrylate of about 1:2:0.2 in Eudragit® RL and about 1:2:0.1 in Eudragit® RS. Preferred insoluble, swellable excipients include crospovidone, croscarmellose, hypromellose acetate succinate (HPMCAS), carbopol, and linear block copolymers of dioxanone and ethylene glycol; linear block copolymers of lactide and ethylene glycol; linear block copolymers of lactide, ethylene glycol, trimethyl carbonate, and caprolactone; linear block copolymers of lactide, glycolide, and ethylene glycol; linear block copolymers of glycolide, polyethylene glycol, and ethylene glycol; such as linear block copolymers of dioxanone (80%) and ethylene glycol (20%); linear block copolymers of lactide (60%) and ethylene glycol (40%); linear block copolymers of lactide (68%), ethylene glycol (20%), trimethyl carbonate (10%), and caprolactone (2%); linear block copolymers of lactide (88%), glycolide (8%), and ethylene glycol (4%); linear block copolymers of glycolide (67%), polyethylene glycol (28%), and ethylene glycol (5%).

Further examples of excipients that can be used in drug eluting components of the gastric residence system are listed in the Excipient Table below.

Excipient Table

| Function | General Examples | Specific Examples |
|---|---|---|
| Polymeric and | Polyalkylene oxides | Kolliphor RH, Kolliphor P407, |
| non-polymeric | Polyethoxylated | Soluplus, Cremophor, SDS |
| solubilizers | castor oil Detergents | |
| Release-enhancing | Acrylate polymers | Eudragit RL |
| excipient (porogen | Acrylate co-polymers | Eudragit RS |
| or wicking agent) | Polyvinylpyrrolidone | Eudragit E |
| | | Linear block copolymer of dioxanone |
| | | and ethlyne glycol (e.g., 80:20 ratio) |
| Dispersant | porous inorganic material | silica, hydrophilic-fumed silica, |
| | polar inorganic material | hydrophobic colloidal silica, |
| | non-toxic metal oxides | magnesium aluminum silicate, stearate |
| | amphiphilic organic | salts, calcium stearate, magnesium |
| | molecules | stearate, microcrystalline cellulose, |
| | polysaccharides, | carboxymethylcellulose, |
| | cellulose, | hypromellose, phospholipids, |
| | cellulose derivatives | polyoxyethylene stearates, zinc |
| | fatty acids | acetate, alginic acid, lecithin, sodium |
| | detergents | lauryl sulfate, aluminum oxide |
| Stabilizer/Preservative | Anti-oxidants | Tocopherols |
| agent | Anti-microbial agents | Alpha-tocopherol |
| | Buffering substances/pH | Ascorbic acid; ascorbate salts |
| | stabilizers | Carotenes |
| | | Butylated hydroxytoluene (BHT) |
| | | Butylated hydroxyanisole (BHA) |
| | | Fumaric acid |
| | | calcium carbonate |
| | | calcium lactate |
| | | calcium phosphate |
| | | sodium phosphate |
| | | sodium bicarbonate |

Table CPE-1 lists combinations of excipients and other additives that can be used in combination with agent and carrier polymer in the drug eluting components of the retention members of a gastric residence system. These excipients and other additives can be combined with agent (where the agent comprises between about 10% to about 60% by weight of the composition), with the carrier polymer, such as polycaprolactone, making up the remainder of the composition. Excipients include the following, which can be used individually or in any combination, in amounts ranging from about 1% to about 30%, such as about 5% to about 20%, by weight of the composition: Kolliphor P407 (poloxamer 407, poly(ethylene glycol)-block-poly(propylene glycol)-block-poly(ethylene glycol)), Eudragit RS (Poly [Ethyl acrylate, methyl methacrylate, trimethylammonioethyl methacrylate chloride] 1:2:0.1), Eudragit RL (Poly [Ethyl acrylate, methyl methacrylate, trimethylammonioethyl methacrylate chloride] 1:2:0.2), PDO (polydioxanone), PEG-PCL, SIF (FaSSIF/FaSSGF powder from BioRelevant), EPO (dimethylaminoethyl methacrylate-butyl methacrylate-methyl methacrylate copolymer), Kollidon VA64 (vinylpyrrolidone-vinyl acetate copolymer in a ratio of 6:4 by mass), polyvinyl acetate, polyvinyl pyrrolidine.

Other additives include silicon dioxide (comprising, for example, about 0.1% to about 5% by weight of the composition, such as about 0.1% to 1% or about 0.5%) and an anti-oxidant, such as alpha-tocopherol (comprising, for example, about 0.1% to about 5% by weight of the composition, such as about 0.1% to 1% or about 0.5%). Each row of the table below represents a formulation of excipients and other additives for use with the carrier polymer and agent.

TABLE CPE-1
Excipients and additives, in combination with
agent or salt thereof and carrier polymer

EPO, P407, Silica, α-tocopherol
EPO, Silica, α-tocopherol
Eudragit RL, Eudragit RS, Kolliphor P407, Silica, α-tocopherol
Eudragit RL, Kolliphor P407, Silica, α-tocopherol
Eudragit RL, Eudragit RS, Kolliphor P407, Silica, α-tocopherol
Eudragit RL, Kolliphor P407, Silica, α-tocopherol
Eudragit RL, Kolliphor P407, Silica, α-tocopherol
Eudragit RS, P407, Silica, α-tocopherol
Eudragit RS, Silica, α-tocopherol
Kollidon VA64, Silica, α-tocopherol
Kolliphor P407, Silica, α-tocopherol
Kolliphor RH40, Silica, α-tocopherol
PDO, Silica, α-tocopherol
PEG-PCL, Silica, α-tocopherol
Poly Vinyl Acetate, Silica, α-tocopherol
PVP, Silica, α-tocopherol
SIF, Silica, α-tocopherol
Silica, P188, P407, α-tocopherol
Silica, α-tocopherol

Table CPE-2 lists specific amounts of excipients and other additives that can be used in combination with agent and carrier polymer in the drug eluting components of the retention members of a gastric residence system.

The amounts listed in Table CPE-2 can be varied by plus-or-minus 20% of each ingredient (for example, 0.5% silica can vary between 0.4% and 0.6% silica, as 20% of 0.5% is 0.1%). Each row of the table below represents a formulation of excipients and other additives for use with the carrier polymer and agent.

TABLE CPE-2
Excipients and additives, in combination with
agent or salt thereof and carrier polymer

0.5% Silica, 0.5% α-tocopherol
0.5% Silica, 2% P407, 0.5% α-tocopherol
0.5% Silica, 2% P188, 2% P407, 0.5% α-tocopherol
0.5% Silica, 3% Eudragit RS, 2% P407, 0.5% α-tocopherol
1% Kolliphor P407, 0.5% Silica, 0.5% α-tocopherol
10% Eudragit RS, 2.5% P407, 2% Silica, 0.5% α-tocopherol
10% Eudragit RS, 5% P407, 0.5% Silica, 0.5% α-tocopherol
10% Eudragit RS, 5% P407, 2% Silica, 0.5% α-tocopherol
12% Eudragit RL, 3% Kolliphor P407, 0.5% Silica, 0.5% α-tocopherol
12% Eudragit RL, 5% Kolliphor P407, 0.5% Silica, 0.5% α-tocopherol
14.78% Eudragit RS, 0.226% P407, 0.5% Silica, 0.5% α-tocopherol
17.5% Eudragit RS, 5% P407, 0.5% Silica, 0.5% α-tocopherol
19.8% Eudragit RS, 0.5% Silica, 0.5% α-tocopherol
2% Kolliphor P407, 0.5% Silica, 0.5% α-tocopherol
2% P407, 0.5% Silica, 0.5% α-tocopherol
20% Eudragit RS, 2% P407, 0.5% Silica, 0.5% α-tocopherol
21.25% Eudragit RS, 2.5% P407, 0.5% Silica, 0.5% α-tocopherol
25% Eudragit RL, 5% P407, 0.5% Silica, 0.5% α-tocopherol
25% Eudragit RS, 0.5% Silica, 0.5% α-tocopherol
25% Eudragit RS, 5% P407, 0.5% Silica, 0.5% α-tocopherol
3% Eudragit RL, 9% Eudragit RS, 5% Kolliphor P407, 0.5% Silica, 0.5%
α-tocopherol
3.5% Eudragit RS, 2.5% P407, 2% Silica, 0.5% α-tocopherol
3.5% Eudragit RS, 5% P407, 2% Silica, 0.5% α-tocopherol
30% PDO, 0.5% Silica, 0.5% α-tocopherol
39.5% PEG-PCL, 0.36% Silica, 0.36% α-tocopherol
4.5% EPO, 4.5% P407, 0.5% Silica, 0.5% α-tocopherol
5% Kolliphor P407, 0.5% Silica, 0.5% α-tocopherol
5% Kolliphor RH40, 0.5% Silica, 0.5% α-tocopherol
5% SIF, 0.5% Silica, 0.5% α-tocopherol
6% Eudragit RL, 5% Kolliphor P407, 0.5% Silica, 0.5% α-tocopherol
6% Eudragit RL, 6% Eudragit RS, 5% Kolliphor P407, 0.5% Silica, 0.5%
α-tocopherol
6.75% Eudragit RS, 3.75% P407, 2% Silica, 0.5% α-tocopherol
7% EPO, 2% P407, 0.5% Silica, 0.5% α-tocopherol
9% EPO, 0.5% Silica, 0.5% α-tocopherol
9% Eudragit RL, 3% Eudragit RS, 5% Kolliphor P407, 0.5% Silica, 0.5%
α-tocopherol
9% Kollidon VA64, 0.5% Silica, 0.5% α-tocopherol
9% Poly Vinyl Acetate, 0.5% Silica, 0.5% α-tocopherol
9% PVP, 0.5% Silica, 0.5% α-tocopherol
9% SIF, 0.5% Silica, 0.5% α-tocopherol

### Drug Eluting Component Composition-Dispersants as Excipients

In some embodiments, an excipient included in the drug eluting component may comprise a dispersant. Using a dispersant in the drug eluting component provides numerous advantages. The rate of elution of agent from the drug eluting component is affected by numerous factors, including the composition and properties of the carrier polymer (which may itself comprise multiple polymeric and non-polymeric components); the physical and chemical properties of the agent; and the gastric environment. Avoiding burst release of agent, especially hydrophilic agents, and maintaining sustained release of the agent over the effective release period or residence period is an important characteristic of the systems. The use of a dispersant enables better control of release rate and suppression of burst release. Burst release and release rate can be tuned by using varied concentrations of dispersant. For example, different dispersants and different excipients, at varying concentrations, can tune burst release of cetirizine in simulated gastric fluid.

Dispersants which can be used in the drug eluting component include: silicon dioxide (silica, SiO2) (hydrophilic fumed); stearate salts, such as calcium stearate and magnesium stearate; microcrystalline cellulose; carboxymethylcellulose; hydrophobic colloidal silica; hypromellose; magnesium aluminum silicate; phospholipids; polyoxyethylene stearates; zinc acetate; alginic acid; lecithin; fatty acids; sodium lauryl sulfate; and non-toxic metal oxides such as aluminum oxide. Porous inorganic materials and polar inorganic materials can be used. Hydrophilic-fumed silicon dioxide is a preferred dispersant. One particularly useful silicon dioxide is sold by Cabot Corporation (Boston, Mass., USA) under the registered trademark CAB-O-SIL® M-5P (CAS #112945-52-5), which is hydrophilic-fumed silicon dioxide having a BET surface area of about 200 m2/g±15 m2/g. The mesh residue for this product on a 45 micron sieve is less than about 0.02%. The typical primary aggregate size is about 150 to about 300 nm, while individual particle sizes may range from about 5 nm to about 50 nm.

In addition to anti-aggregation/anti-flocculation activity, the dispersant can help prevent phase separation during fabrication and/or storage of a gastric residence system. This is particularly useful for manufacture of the systems by hot melt extrusion.

The weight/weight ratio of dispersant to agent substance can be about 0.1% to about 5%, about 0.1% to about 4%, about 0.1% to about 3%, about 0.1% to about 2%, about 0.1% to about 1%, about 1% to about 5%, about 1% to about 4%, about 1% to about 3%, about 1% to about 2%, about 2% to about 4%, about 2% to about 3%, about 3% to about 4%, about 4% to about 5%, or about 0.1%, about 0.5%, about 1%, about 2%, about 3%, about 4% or about 5%.

Dispersants can comprise about 0.1% to about 4% of the drug eluting component, such as about 0.1% to about 3.5%, about 0.1% to about 3%, about 0.1% to about 2.5%, about 0.1% to about 2%, about 0.1% to about 1.5%, about 0.1% to about 1%, about 0.1% to about 0.5%, or about 0.2% to about 0.8%.

Dispersants can also be used to modulate the amount of burst release of agent during the initial period when a gastric residence system is administered. In embodiments of a gastric residence system that is to be administered once weekly, the burst release over the approximately first six hours after initial administration is less than about 8%, preferably less than about 6%, of the total amount of agent in the system. In embodiments of a gastric residence system that is to be administered once every three days, the burst release over the approximately first six hours after initial administration is less than about 12%, preferably less than about 10%, of the total amount of agent in the system. In embodiments of a gastric residence system that is to be administered once daily, the burst release over the approximately first six hours after initial administration is less than about 40%, preferably less than about 30%, of the total amount of agent in the system. In general, if a new gastric residence system is administered every D days, and the total mass of agent is M, then the gastric residence system releases less than about [(M divided by D) times 0.5], preferably less than about [(M divided by D) multiplied by 0.4], or less than about [(M divided by D) multiplied by ⅜], more preferably less than about [(M divided by D) multiplied by 0.3], over the approximately first six hours after initial administration. In further embodiments, the gastric residence system releases at least about [(M divided by D) multiplied by 0.25] over the approximately first six hours after initial administration, that is, the system releases at least about one-quarter of the daily dosage over the first one-quarter of the first day of administration.

### Drug Eluting Component Composition—Stabilizers as Excipients

In some embodiments, an excipient included in the drug eluting component may comprise a stabilizer. Many agents are prone to oxidative degradation when exposed to reactive oxygen species, which can be present in the stomach. An agent contained in the drug eluting component of a gastric residence system may thus oxidize due to the prolonged residence in the stomach of the system, and the extended release period of agent from the system. Accordingly, it is desirable to include stabilizers or preservatives in the systems, in order to stabilize the agent to prevent oxidative and other degradation.

Stabilizers, such as anti-oxidants including tocopherols, alpha-tocopherol, ascorbic acid, ascorbyl palmitate, butylated hydroxytoluene, butylated hydroxyanisole, and fumaric acid, can comprise about 0.1% to about 4% of the drug eluting components, such as about 0.1% to about 3.5%, about 0.1% to about 3%, about 0.1% to about 2.5%, about 0.1% to about 2%, about 0.1% to about 1.5%, about 0.1% to about 1%, about 0.1% to about 0.5%, or about 0.2% to about 0.8%.

Anti-oxidant stabilizers that can be included in the gastric residence systems described herein to reduce or prevent oxidation of the agent include alpha-tocopherol (about 0.01 to about 0.05% v/v), ascorbic acid (about 0.01 to about 0.1% w/v), ascorbyl palmitate (about 0.01 to about 0.1% w/v), butylated hydroxytoluene (about 0.01 to about 0.1% w/w), butylated hydroxyanisole (about 0.01 to about 0.1% w/w), and fumaric acid (up to 3600 ppm). Vitamin E, a tocopherol, a Vitamin E ester, a tocopherol ester, ascorbic acid, or a carotene, such as alpha-tocopherol, Vitamin E succinate, alpha-tocopherol succinate, Vitamin E acetate, alpha-tocopherol acetate, Vitamin E nicotinate, alpha-tocopherol nicotinate, Vitamin E linoleate, or alpha-tocopherol linoleate can be used as anti-oxidant stabilizers.

Certain agents can be pH-sensitive, especially at the low pH present in the gastric environment. Buffering or pH-stabilizer compounds that can be included in the systems to reduce or prevent degradation of agent at low pH include calcium carbonate, calcium lactate, calcium phosphate, sodium phosphate, and sodium bicarbonate. Buffering or pH-stabilizer compounds are typically used in an amount of up to about 2% w/w. The buffering or pH-stabilizer compounds can comprise about 0.1% to about 4% of the drug eluting components, such as about 0.1% to about 3.5%, about 0.1% to about 3%, about 0.1% to about 2.5%, about 0.1% to about 2%, about 0.1% to about 1.5%, about 0.1% to about 1%, about 0.1% to about 0.5%, or about 0.2% to about 0.8%.

The anti-oxidant stabilizers, pH stabilizers, and other stabilizer compounds are blended into the polymers containing the agent by blending the stabilizer(s) into the molten carrier polymer-agent mixture. The stabilizer(s) can be blended into molten carrier polymer prior to blending the agent into the polymer-stabilizer mixture; or the stabilizer(s) can be blended with agent prior to formulation of the blended agent-stabilizer mixture in the carrier polymer; or stabilizer(s), agent, and molten carrier polymer can be blended simultaneously. Agent can also be blended with molten carrier polymer prior to blending the stabilizer(s) into the polymer-agent mixture.

In one embodiment, less than about 10% of the agent remaining in the gastric residence system is degraded or oxidized after a gastric residence period of about 24 hours. In one embodiment, less than about 10% of the agent remaining in the system is degraded or oxidized after a gastric residence period of about 48 hours. In one embodiment, less than about 10% of the agent remaining in the system is degraded or oxidized after a gastric residence period of about 72 hours. In one embodiment, less than about 10% of the agent remaining in the system is degraded or oxidized after a gastric residence period of about 96 hours. In one embodiment, less than about 10% of the agent remaining in the system is degraded or oxidized after a gastric residence period of about five days. In some embodiments, less than about 10% of the agent remaining in the system is degraded or oxidized after a gastric residence period of about a week. In some embodiments, less than about 10% of the agent remaining in the system is degraded or oxidized after a gastric residence period of about two weeks.

In one embodiment, less than about 5% of the agent remaining in the gastric residence system is degraded or oxidized after a gastric residence period of about 24 hours. In one embodiment, less than about 5% of the agent remaining in the system is degraded or oxidized after a gastric residence period of about 48 hours. In one embodiment, less than about 5% of the agent remaining in the system is degraded or oxidized after a gastric residence period of about 72 hours. In one embodiment, less than about 5% of the agent remaining in the system is degraded or oxidized after a gastric residence period of about 96 hours. In one embodiment, less than about 5% of the agent remaining in the system is degraded or oxidized after a gastric residence period of about five days. In some embodiments, less than about 5% of the agent remaining in the system is degraded or oxidized after a gastric residence period of about a week. In some embodiments, less than about 5% of the agent remaining in the system is degraded or oxidized after a gastric residence period of about two weeks.

### Laser Linker Component Compositions

A retention member of a gastric residence system may include one or more laser linker components. Laser linker components can be laser welded to one or more drug eluting components, neighboring laser linker components, or an elastomeric component. In some embodiments, laser linker components may be or may include inactive components (e.g., inert components used to maintain consistent geometry and size of the gastric residence system) or disintegrating matrices such as time dependent disintegrating matrices or enteric disintegrating matrices.

### Laser Linker Component Compositions—Enteric and Time-Dependent Disintegrating Matrices

As described above, laser linker components may be used to connect one or more drug eluting components to one or more elastomeric components, additional drug eluting components, or additional laser linker components; or to connect one or more laser linker components to one or more drug eluting components, one or more additional laser linker components, or one or more elastomeric components. Thus, laser linker components connect various components of the system (e.g., various components of retention members). In some embodiments, a laser linker component may include one or more enteric or time-dependent disintegrating matrices. A disintegrating matrix may include one or more enteric polymers which are designed to break down gradually in a controlled manner during the residence period of the system in the stomach and/or one or more time-dependent polymers which are pH-resistant, that is, less sensitive to changes in pH than enteric polymers. In some embodiments, both enteric polymers and time-dependent polymers which are less sensitive to changes in pH than enteric polymers may be used.

In some embodiments, a laser linker component may include an enteric disintegrating matrix comprising an enteric polymer which is designed to break down gradually in a controlled manner during the residence period of the system in the stomach. If the gastric residence system passes prematurely into the small intestine in an intact form, the system is designed to break down much more rapidly to avoid intestinal obstruction. Enteric polymers are relatively resistant to the acidic pH levels encountered in the stomach, but dissolve at the higher pH levels found in the duodenum. Use of enteric polymers as safety elements protects against undesired passage of the intact gastric residence system into the small intestine.

Enteric polymers are relatively insoluble under acidic conditions, such as the conditions encountered in the stomach, but are soluble under the less acidic to basic conditions encountered in the small intestine. Enteric polymers which dissolve at about pH 5 or above can be used in an enteric disintegrating matrix component, as the pH of the initial segment of the small intestine, the duodenum, ranges from about 5.4 to 6.1. If the gastric residence system passes intact through the pyloric valve, the enteric polymer will dissolve and the components linked by the enteric polymer will break apart, allowing passage of the residence system through the small and large intestines. Thus, the gastric residence systems are designed to uncouple rapidly in the intestinal environment by dissolution of the enteric polymer.

Exemplary enteric polymers include, but are not limited to, cellulose acetate phthalate, cellulose acetate succinate, methylcellulose phthalate, ethylhydroxycellulose phthalate, polyvinylacetatephthalate, polyvinylbutyrate acetate, vinyl acetate-maleic anhydride copolymer, styrene-maleic mono-ester copolymer, methacrylic acid methylmethacrylate copolymer, methyl acrylate-methacrylic acid copolymer, methacrylate-methacrylic acid-octyl acrylate copolymer, and copolymers, mixtures, blends and combinations thereof. Some of the enteric polymers that can be used in the invention are listed in the Enteric Polymer Table, along with their dissolution pH. (See Mukherji, Gour and Clive G. Wilson, “Enteric Coating for Colonic Delivery,” Chapter 18 of Modified-Release Drug Delivery Technology (editors Michael J. Rathbone, Jonathan Hadgraft, Michael S. Roberts), Drugs and the Pharmaceutical Sciences Volume 126, New York: Marcel Dekker, 2002.) Preferably, enteric polymers that dissolve at a pH of no greater than about 5 or about 5.5 are used. Poly(methacrylic acid-co-ethyl acrylate) (sold under the trade name EUDRAGIT L 100-55; EUDRAGIT is a registered trademark of Evonik Rohm GmbH, Darmstadt, Germany) is a preferred enteric polymer. Another preferred enteric polymer is hydroxypropylmethylcellulose acetate succinate (hypromellose acetate succinate or HPMCAS; Ashland, Inc., Covington, Ky., USA), which has a tunable pH cutoff from about 5.5 to about 7.0. Cellulose acetate phthalate, cellulose acetate succinate, and hydroxypropyl methylcellulose phthalate are also suitable enteric polymers.

In one embodiment, the enteric polymers used in the gastric residence system dissolve at a pH above about 4. In some embodiments, the enteric polymers used in the gastric residence system dissolve at a pH above about 5. In some embodiments, the enteric polymers used in the gastric residence system dissolve at a pH above about 6. In some embodiments, the enteric polymers used in the gastric residence system dissolve at a pH above about 7. In some embodiments, the enteric polymers used in the gastric residence system dissolve at a pH above about 7.5. In some embodiments, the enteric polymers used in the gastric residence system dissolve at a pH between about 4 and about 5. In some embodiments, the enteric polymers used in the gastric residence system dissolve at a pH between about 4 and about 6. In some embodiments, the enteric polymers used in the gastric residence system dissolve at a pH between about 4 and about 7. In some embodiments, the enteric polymers used in the gastric residence system dissolve at a pH between about 4 and about 7.5. In some embodiments, the enteric polymers used in the gastric residence system dissolve at a pH between about 5 and about 6. In some embodiments, the enteric polymers used in the gastric residence system dissolve at a pH between about 5 and about 7. In some embodiments, the enteric polymers used in the gastric residence system dissolve at a pH between about 5 and about 7.5. In some embodiments, the enteric polymers used in the gastric residence system dissolve at a pH between about 6 and about 7. In some embodiments, the enteric polymers used in the gastric residence system dissolve at a pH between about 6 and about 7.5.

Enteric Polymer Table

| Polymer | Dissolution pH |
|---|---|
| Cellulose acetate phthalate | 6.0-6.4 |
| Hydroxypropyl methylcellulose phthalate 50 | 4.8 |
| Hydroxypropyl methylcellulose phthalate 55 | 5.2 |
| Polyvinylacetate phthalate | 5.0 |
| Methacrylic acid-methyl methacrylate | 6.0 |
| copolymer (1:1) | |
| Methacrylic acid-methyl methacrylate | 6.5-7.5 |
| copolymer (2:1) | |
| Methacrylic acid-ethyl acrylate copolymer | 5.5 |
| (2:1) | |
| Shellac | 7.0 |
| Hydroxypropyl methylcellulose acetate | 7.0 |
| succinate | |
| Poly (methyl vinyl ether/maleic acid) | 4.5-5.0 |
| monoethyl ester | |
| Poly (methyl vinyl ether/maleic acid) n-butyl | 5.4 |
| ester | |

In some embodiments, retention members of a gastric residence system are comprised of multiple segments attached by enteric disintegrating matrices. In some embodiments, a drug eluting component of a gastric residence system is attached to an elastomeric component of the system by one or more enteric disintegrating matrices. In any of these embodiments, when enteric disintegrating matrices are used for both segment-to-segment attachments and for attachment of the retention members to an elastomeric component, the enteric polymer used for segment-segment attachments can be the same enteric polymer as the enteric polymer used for attachment of the retention members to the elastomeric component, or the enteric polymer used for segment-segment attachments can be a different enteric polymer than the enteric polymer used for attachment of the retention members to the elastomeric component. The enteric polymers used for the segment-segment attachments can all be the same enteric polymer, or can all be different enteric polymers, or some enteric polymers in the segment-segment attachments can be the same and some enteric polymers in the segment-segment attachments can be different. That is, the enteric polymer(s) used for each segment-segment attachment and the enteric polymer used for attachment of the retention members to the elastomeric component can be independently chosen.

In some embodiments, a gastric residence system may include one or more time-dependent disintegrating matrices instead of or in addition to one or more enteric disintegrating matrices. A time-dependent disintegrating matrix may include one or more time-dependent polymers or linkers, that is, polymers that degrade in a time-dependent manner in the gastric environment. For example, the liquid plasticizer triacetin releases from a polymer formulation in a time-dependent manner over seven days in simulated gastric fluid, while Plastoid B retains its strength over a seven-day period in simulated gastric fluid. Thus, a polymer that degrades in a time-dependent manner can be readily prepared by mixing Plastoid B and triacetin; the degradation time of the Plastoid B-triacetin mixture can be extended by increasing the amount of Plastoid B used in the mixture (that is, using less triacetin in the mixture), while the degradation time can be decreased by decreasing the amount of Plastoid B used in the mixture (that is, using more triacetin in the mixture).

A time-dependent polymer or linker degrades in a predictable, time-dependent manner. In some embodiments, the degradation of the time-dependent polymer or linker may not be affected by the varying pH of the gastrointestinal system. By “time-dependent polymer which are pH-resistant” (or equivalently, “pH-resistant time-dependent polymers”) is meant that, under conditions where an enteric polymer would degrade to the point that it would no longer link the components together, the time-dependent polymer will still have sufficient mechanical strength to link the components together. In some embodiments, the time-dependent polymer retains about the same linking capacity, that is, about 100% of its linkage strength, after exposure to a solution between about pH 7 to about pH 8 as it has after exposure to a solution between about pH 2 to about pH 3, where the exposure is for about an hour, about a day, about three days, or about a week. In some embodiments, the time-dependent polymer retains at least about 90% of its linkage strength, after exposure to a solution between about pH 7 to about pH 8 as it has after exposure to a solution between about pH 2 to about pH 3, where the exposure is for about an hour, about a day, about three days, or about a week. In some embodiments, the time-dependent polymer retains at least about 75% of its linkage strength, after exposure to a solution between about pH 7 to about pH 8 as it has after exposure to a solution between about pH 2 to about pH 3, where the exposure is for about an hour, about a day, about three days, or about a week. In some embodiments, the time-dependent polymer retains at least about 60% of its linkage strength, after exposure to a solution between about pH 7 to about pH 8 as it has after exposure to a solution between about pH 2 to about pH 3, where the exposure is for about an hour, about a day, about three days, or about a week. In some embodiments, the time-dependent polymer retains at least about 50% of its linkage strength, after exposure to a solution between about pH 7 to about pH 8 as it has after exposure to a solution between about pH 2 to about pH 3, where the exposure is for about an hour, about a day, about three days, or about a week. In some embodiments, the time-dependent polymer retains at least about 25% of its linkage strength, after exposure to a solution between about pH 7 to about pH 8 as it has after exposure to a solution between about pH 2 to about pH 3, where the exposure is for about an hour, about a day, about three days, or about a week. In some embodiments, the time-dependent polymer resists breaking under a flexural force of about 0.2 Newtons (N), about 0.3 N, about 0.4 N, about 0.5 N, about 0.75 N, about 1 N, about 1.5 N, about 2 N, about 2.5 N, about 3 N, about 4 N, or about 5 N, after exposure to a solution between about pH 7 to about pH 8, where the exposure is for about an hour, about a day, about three days, or about a week. Linkage strength can be measured by any relevant test that serves to test coupling ability, such as a four-point bending flexural test (ASTM D790).

A variety of time-dependent mechanisms are available. Water-soluble time-dependent polymers break down as water penetrates through the polymer. Examples of such polymers are hydroxypropyl methylcellulose and poly vinyl acetate. Acid soluble time-dependent polymers break down over time in an acidic environment. Examples include Eudragit EPO. Time-dependent polymers can use water soluble plasticizers; as plasticizer is released, the remaining polymer becomes brittle and breaks under gastric forces. Examples of such polymers include triacetin and triethyl citrate.

In any of the embodiments of the gastric residence systems described herein, the enteric or time-dependent polymers or linkers can comprise hydroxypropyl methyl cellulose acetate succinate (HPMCAS) and polycaprolactone (PCL). These blends can be used to form disintegrating linkers or disintegrating matrices. The ratio of HPMCAS to polycaprolactone in a disintegrating matrix can be between about 80% HPMCAS: 20% PCL to about 20% HPMCAS: 80% PCL. The ratio of HPMCAS to polycaprolactone can be between about 80% HPMCAS: 20% PCL to about 20% HPMCAS: 80% PCL; between about 70% HPMCAS: 30% PCL to about 30% HPMCAS: 70% PCL; between about 60% HPMCAS: 40% PCL to about 40% HPMCAS: 60% PCL; between about 80% HPMCAS: 20% PCL to about 50% HPMCAS: 50% PCL; between about 80% HPMCAS: 20% PCL to about 60% HPMCAS: 40% PCL; between about 70% HPMCAS: 30% PCL to about 50% HPMCAS: 50% PCL; between about 70% HPMCAS: 30% PCL to about 60% HPMCAS: 40% PCL; between about 20% HPMCAS: 80% PCL to about 40% HPMCAS: 60% PCL; between about 20% HPMCAS: 80% PCL to about 50% HPMCAS: 50% PCL; between about 30% HPMCAS: 70% PCL to about 40% HPMCAS: 60% PCL; between about 30% HPMCAS: 70% PCL to about 50% HPMCAS: 50% PCL; or about 80% HPMCAS: 20% PCL, about 70% HPMCAS: 30% PCL, about 60% HPMCAS: 40% PCL, about 50% HPMCAS: 50% PCL, about 40% HPMCAS: 60% PCL, about 30% HPMCAS: 70% PCL, or about 20% HPMCAS: 80% PCL. The disintegrating matrix can further comprise a plasticizer selected from the group consisting of triacetin, triethyl citrate, tributyl citrate, poloxamers, polyethylene glycol, polypropylene glycol, diethyl phthalate, dibutyl sebacate, glycerin, castor oil, acetyl triethyl citrate, acetyl tributyl citrate, polyethylene glycol monomethyl ether, sorbitol, sorbitan, a sorbitol-sorbitan mixture, and diacetylated monoglycerides.

The compositions of the disintegrating matrices are chosen to weaken sufficiently after a specified period of time in order to allow the gastric residence systems to reach a point where they de-couple and pass through the pylorus and out of the stomach after the desired residence period or weaken sufficiently such that the gastric residence system is no longer retained in the stomach; that is, the disintegrating matrices weaken to the point of uncoupling (the uncoupling point) or to the point where the gastric residence system can pass through the pylorus (the pyloric passage point, or passage point). Thus, in one embodiment, disintegrating matrices are used that uncouple after about two days in a human stomach; after about three days in a human stomach; after about four days in a human stomach; after about five days in a human stomach; after about six days in a human stomach; after about seven days in a human stomach; after about eight days in a human stomach; after about nine days in a human stomach; after about ten days in a human stomach; or after about two weeks in a human stomach. In one embodiment, disintegrating matrices are used that uncouple after about two days in a dog stomach; after about three days in a dog stomach; after about four days in a dog stomach; after about five days in a dog stomach; after about six days in a dog stomach; after about seven days in a dog stomach; after about eight days in a dog stomach; after about nine days in a dog stomach; after about ten days in a dog stomach; or after about two weeks in a dog stomach. In one embodiment, disintegrating matrices are used that uncouple after about two days in a pig stomach; after about three days in a pig stomach; after about four days in a pig stomach; after about five days in a pig stomach; after about six days in a pig stomach; after about seven days in a pig stomach; after about eight days in a pig stomach; after about nine days in a pig stomach; after about ten days in a pig stomach; or after about two weeks in a pig stomach. In one embodiment, disintegrating matrices are used that uncouple after about two days in fasted-state simulated gastric fluid; after about three days in fasted-state simulated gastric fluid; after about four days in fasted-state simulated gastric fluid; after about five days in fasted-state simulated gastric fluid; after about six days in fasted-state simulated gastric fluid; after about seven days in fasted-state simulated gastric fluid; after about eight days in fasted-state simulated gastric fluid; after about nine days in fasted-state simulated gastric fluid; after about ten days in fasted-state simulated gastric fluid; or after about two weeks in fasted-state simulated gastric fluid. In one embodiment, disintegrating matrices are used that uncouple after about two days in fed-state simulated gastric fluid; after about three days in fed-state simulated gastric fluid; after about four days in fed-state simulated gastric fluid; after about five days in fed-state simulated gastric fluid; after about six days in fed-state simulated gastric fluid; after about seven days in fed-state simulated gastric fluid; after about eight days in fed-state simulated gastric fluid; after about nine days in fed-state simulated gastric fluid; after about ten days in fed-state simulated gastric fluid; or after about two weeks in fed-state simulated gastric fluid. In one embodiment, disintegrating matrices are used that uncouple after about two days in water at pH 2; after about three days in water at pH 2; after about four days in water at pH 2; after about five days in water at pH 2; after about six days in water at pH 2; after about seven days in water at pH 2; after about eight days in water at pH 2; after about nine days in water at pH 2; after about ten days in water at pH 2; or after about two weeks in water at pH 2. In one embodiment, disintegrating matrices are used that uncouple after about two days in water at pH 1; after about three days in water at pH 1; after about four days in water at pH 1; after about five days in water at pH 1; after about six days in water at pH 1; after about seven days in water at pH 1; after about eight days in water at pH 1; after about nine days in water at pH 1; after about ten days in water at pH 1; or after about two weeks in water at pH 1.

The de-coupling or pyloric passage point in human, dog, or pig occurs when the system passes out of the stomach, that is, when it passes through the pylorus. For the in vitro measurements in simulated gastric fluid or acidic water, the de-coupling or pyloric passage point occurs when the disintegrating matrix weakens to the point where it will break under the normal compressive forces of the stomach, typically about 0.1 Newton to 0.2 Newton. Linkage strength (breaking point) can be measured by any relevant test that serves to test coupling ability, that is, the force required to break the linker, such as the four-point bending flexural test (ASTM D790) described in Example 18 of WO 2017/070612, or Examples 12, 13, 15, 17, or 18 of WO 2017/100367. In one embodiment, the de-coupling or pyloric passage point is reached when the disintegrating matrices uncouple at about 0.2 N of force. In another embodiment, the de-coupling or pyloric passage point is reached when the disintegrating matrices uncouple at about 0.1 N of force.

The gastric residence systems can reach the pyloric passage point without any or all of the disintegrating matrices actually breaking. If the disintegrating matrices weaken or degrade to the point where they can no longer hold the gastric residence system in the stomach, even if one, some, or all of the disintegrating matrices do not break, the gastric residence system will pass through the pylorus and into the small intestine (the pyloric passage point or passage point). In some embodiments, disintegrating matrices are used that weaken to the passage point after about two days in a human stomach; after about three days in a human stomach; after about four days in a human stomach; after about five days in a human stomach; after about six days in a human stomach; after about seven days in a human stomach; after about eight days in a human stomach; after about nine days in a human stomach; after about ten days in a human stomach; or after about two weeks in a human stomach. In some embodiments, disintegrating matrices are used that weaken to the passage point after about two days in a dog stomach; after about three days in a dog stomach; after about four days in a dog stomach; after about five days in a dog stomach; after about six days in a dog stomach; after about seven days in a dog stomach; after about eight days in a dog stomach; after about nine days in a dog stomach; after about ten days in a dog stomach; or after about two weeks in a dog stomach. In some embodiments, disintegrating matrices are used that weaken to the passage point after about two days in a pig stomach; after about three days in a pig stomach; after about four days in a pig stomach; after about five days in a pig stomach; after about six days in a pig stomach; after about seven days in a pig stomach; after about eight days in a pig stomach; after about nine days in a pig stomach; after about ten days in a pig stomach; or after about two weeks in a pig stomach. In some embodiments, disintegrating matrices are used that weaken to the passage point after about two days in fasted-state simulated gastric fluid; after about three days in fasted-state simulated gastric fluid; after about four days in fasted-state simulated gastric fluid; after about five days in fasted-state simulated gastric fluid; after about six days in fasted-state simulated gastric fluid; after about seven days in fasted-state simulated gastric fluid; after about eight days in fasted-state simulated gastric fluid; after about nine days in fasted-state simulated gastric fluid; after about ten days in fasted-state simulated gastric fluid; or after about two weeks in fasted-state simulated gastric fluid. In some embodiments, disintegrating matrices are used that weaken to the passage point after about two days in fed-state simulated gastric fluid; after about three days in fed-state simulated gastric fluid; after about four days in fed-state simulated gastric fluid; after about five days in fed-state simulated gastric fluid; after about six days in fed-state simulated gastric fluid; after about seven days in fed-state simulated gastric fluid; after about eight days in fed-state simulated gastric fluid; after about nine days in fed-state simulated gastric fluid; after about ten days in fed-state simulated gastric fluid; or after about two weeks in fed-state simulated gastric fluid. In some embodiments, disintegrating matrices are used that weaken to the passage point after about two days in water at pH 2; after about three days in water at pH 2; after about four days in water at pH 2; after about five days in water at pH 2; after about six days in water at pH 2; after about seven days in water at pH 2; after about eight days in water at pH 2; after about nine days in water at pH 2; after about ten days in water at pH 2; or after about two weeks in water at pH 2. In some embodiments, disintegrating matrices are used that weaken to the passage point after about two days in water at pH 1; after about three days in water at pH 1; after about four days in water at pH 1; after about five days in water at pH 1; after about six days in water at pH 1; after about seven days in water at pH 1; after about eight days in water at pH 1; after about nine days in water at pH 1; after about ten days in water at pH 1; or after about two weeks in water at pH 1.

### Laser Linker Component Compositions—Inactive Components

In some embodiments, laser linker components may comprise one or more inactive components used as spacers or used to otherwise maintain the shape and size of a gastric residence system. In some embodiments, an inactive component may comprise a common polymer with other segments (e.g., drug eluting components) in the gastric residence system. Suitable polymers may include thermoplastic co-polyesters, polycaprolactone (PCL), thermoplastic polyurethanes, thermoplastic polyamides, thermoplastic vulcanizates, styrenic block copolymers, polyethylene-co-vinyl acetate, or polylactic acid.

In some embodiments, a common polymer included in an inactive segment comprises polycaprolactone (PCL). In some embodiments, an inactive component comprises about 60-85 wt. % PCL, about 65-75 wt. % PCL, or about 70 wt. % PCL. In some embodiments, an inactive component comprises about 61-71 wt. % PCL, about 64-69 wt. % PCL, or about 66.45 wt. % PCL. In some embodiments, an inactive component comprises pure PCL.

In some embodiments, an inactive component may include a radiopaque substance. Adding a radiopaque substance may aid in locating a gastric residence system via abdominal X-ray if necessary. Examples of suitable radiopaque substances are barium sulfate, bismuth subcarbonate, bismuth oxychloride, and bismuth trioxide. In some embodiments, an inactive component may comprise bismuth subcarbonate. In some embodiments, an inactive component comprises about 15-40 wt. % bismuth subcarbonate, about 25-35 wt. % bismuth subcarbonate, or about 30 wt. % bismuth subcarbonate.

In some embodiments, an inactive component may include vinylpyrrolidone-vinyl acetate copolymer in a ratio of 6:4 by mass (i.e., copovidone, such as Kollidon VA64). In some embodiments, an inactive component comprises about 27-37 wt. % copovidone, about 30-34 wt. % copovidone, or about 32 wt. % copovidone.

In some embodiments, an inactive component may include one or more plasticizers, such as a poloxamer (e.g., Poloxamer 407, or “P407”). In some embodiments, an inactive component comprises about 0.2-4 wt. % poloxamer, about 0.5-2.5 wt. % poloxamer, or about 1.50 wt. % poloxamer.

In some embodiments, an inactive component may include a color-absorbing dye (also referred to as a colorant or a pigment). For example, an inactive component may include FD&C Blue 1 Aluminum Lake, 11-13%. In some embodiments, an inactive component comprises about 0.005-0.2 wt. % color-absorbing dye, about 0.01-0.1 wt. % color-absorbing dye, or about 0.05 wt. % color-absorbing dye.

Exemplary amounts of the components for a first embodiment of an inactive component are provided in the table below. The amounts are given in an approximate weight percent, with the understanding that when ranges are provided, the amounts are chosen so as to add up to 100%.

| Component | Formulation 1 | Formulation 2 | Formulation 3 |
|---|---|---|---|
| PCL | 60-85 | 65-75 | 70 |
| Bismuth | 15-40 | 25-35 | 30 |
| subcarbonate | | | |

Exemplary amounts of the components for another embodiment of an inactive component are provided in the table below. The amounts are given in an approximate weight percent, with the understanding that when ranges are provided, the amounts are chosen so as to add up to 100%.

| Component | Formulation 1 | Formulation 2 | Formulation 3 |
|---|---|---|---|
| PCL | 61-71 | 64-69 | 66.45 |
| VA64 | 27-37 | 30-34 | 32 |
| P407 | 0.2-4 | 0.5-2.5 | 1.5 |
| Color-absorbing dye | 0.005-0.2 | 0.01-0.1 | 0.05 |

### Elastomeric Component

In some embodiments, a gastric residence system may include one or more elastomeric components. An elastomeric component may comprise one or more elastomers, also referred to as elastic polymers or tensile polymers, which enable the gastric residence system to be compacted, such as by being folded or compressed, into a form suitable for administration to the stomach by swallowing a container or capsule containing the compacted system. Upon dissolution of the capsule in the stomach, the gastric residence system expands into a shape which prevents passage of the system through the pyloric sphincter of the patient for the desired residence time of the system. Thus, the elastomer must be capable of being stored in a compacted configuration in a capsule for a reasonable shelf life, and of expanding to its original shape, or approximately its original shape, upon release from the capsule.

In some embodiments, the elastomer is a silicone elastomer. The elastomer may be formed from a liquid silicone rubber (LSR), such as sold in the Dow Corning QP-1 liquid silicone rubber kit. In some embodiments, the elastomer is crosslinked polycaprolactone. In one embodiment, the elastomer is an enteric polymer, such as those listed in the Enteric Polymer Table. (The elastomeric component of a stellate system is typically not an enteric polymer; however, the elastomeric component can also be made from such an enteric polymer where desirable and practical.) Other examples of elastomers which can be used include poly(acryloyl 6-aminocaproic acid) (PA6ACA); poly(methacrylic acid-co-ethyl acrylate) (EUDRAGIT L 100-55); and mixtures of poly(acryloyl 6-aminocaproic acid) (PA6ACA) and poly(methacrylic acid-co-ethyl acrylate) (EUDRAGIT L 100-55).

The elastomeric component may have a specific durometer and compression set. The durometer may determine the folding force of the dosage form and whether it will remain in the stomach; a preferred range is from about 60 to about 90A. The compression set may be low in order to avoid permanently deforming the gastric residence system when stored in the capsule in its compacted configuration. A preferred range is about 10% to about 20% range. Examples of materials that fit these requirements are the QP1 range of liquid silicone rubbers from Dow Corning. In any embodiment with an elastomeric component, the QP1-270 (70A durometer) liquid silicone rubber can be used. In some embodiments, the elastomeric component may comprise a 50A or 60A durometer liquid silicone rubber (Shin Etsu).

In some embodiments, a stellate-shaped gastric residence system may comprise a central elastomeric component and a plurality of retention members attached to the elastomeric component. The proximal end of each retention member may be attached to the elastomeric component and project radially from the elastomeric component, such that each retention member has its distal end not attached to the elastomeric component and located at a larger radial distance from the elastomeric component than the proximal end. In some embodiments, six retention members may be attached to an elastomeric component in a stellate configuration, but three, four, five, seven, eight, nine, or ten retention members can be used. The retention members may be equally spaced around the elastomeric component; if there are N retention members, there will be an angle of about 360/N degrees between neighboring retention members.

### System Polymeric Composition

The choice of the individual polymers for the carrier polymer, enteric and/or time-dependent polymer, and elastomer influence many properties of the system, such as drug elution rate (dependent on the carrier polymer, as well as other factors), the residence time of the system (dependent on the degradation of any of the polymers, principally the enteric or time-dependent polymers), the uncoupling time of the system if it passes into the intestine (dependent primarily on the enteric degradation rate of the enteric and/or time-dependent polymer, as discussed herein), and the shelf life of the system in its compressed form (dependent primarily on properties of the elastomer). As the systems will be administered to the gastrointestinal tract, all of the system components should be biocompatible with the gastrointestinal environment.

The rate of elution of drug from the drug eluting component is affected by numerous factors, including the composition and properties of the carrier polymer, which may itself be a mixture of several polymeric and non-polymeric components; the properties of the drug such as hydrophilicity/hydrophobicity, charge state, pKa, and hydrogen bonding capacity; and the properties of the gastric environment. In the aqueous environment of the stomach, avoiding burst release of a drug (where burst release refers to a high initial delivery of active pharmaceutical ingredient upon initial deployment of the system in the stomach), particularly a hydrophilic drug, and maintaining sustained release of the drug over a period of time of days to one or two weeks is challenging.

The residence time of the systems in the stomach is adjusted by the choice of enteric and/or time-dependent polymers used in the laser linker components. The systems will eventually break down in the stomach, despite the use of enteric polymers, as the mechanical action of the stomach and fluctuating pH will eventually weaken the enteric polymers. Polymers which degrade in a time-dependent manner in the stomach can also be used to adjust the time until the system breaks apart or weakens to the point where it can no longer resist passage through the pylorus, and hence adjust the residence time. Once the system breaks apart or weakens to the point where it can no longer resist passage through the pylorus, it passes into the intestines and is then eliminated.

The elastomer used in the systems is central to the shelf life of the systems. When the systems are compressed, the elastomer is subjected to mechanical stress. The stress in turn can cause polymer creep, which, if extensive enough, can prevent the systems from returning to their uncompacted configurations when released from the capsules or other container; this in turn would lead to premature passage of the system from the stomach. Polymer creep can also be temperature dependent, and therefore the expected storage conditions of the systems also need to be considered when choosing the elastomer and other polymer components.

The system components and polymers should not swell, or should have minimal swelling, in the gastric environment. The components should swell no more than about 20%, no more than about 10%, or no more than about 5% when in the gastric environment over the period of residence.

The systems are optionally radiopaque, so that they can be located via abdominal X-ray if necessary. In some embodiments, one or more of the materials used for construction of the system is sufficiently radiopaque for X-ray visualization. In other embodiments, a radiopaque substance is added to one or more materials of the system, or coated onto one or more materials of the system, or are added to a small portion of the system. Examples of suitable radiopaque substances are barium sulfate, bismuth subcarbonate, bismuth oxychloride, and bismuth trioxide. These materials are typically blended into a separate piece of the gastric residence system, such as a small segment in one or more of the retention members, so as not to alter drug release from the carrier polymer, or desired properties of other system polymers such as the linkers or elastomeric component. Metal striping or tips on a small portion of the system components can also be used, such as tungsten.

### Exemplary Gastric Residence System Compositions

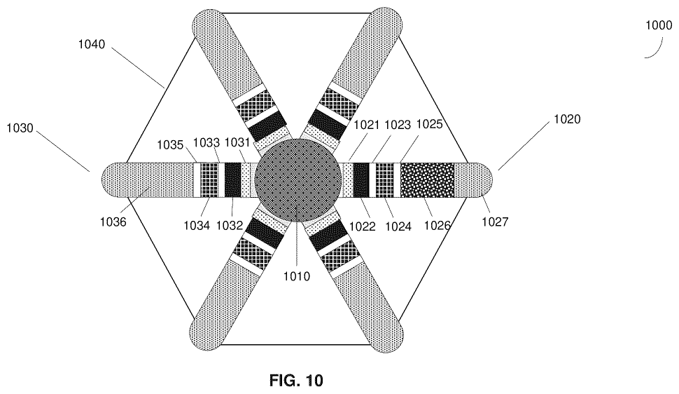

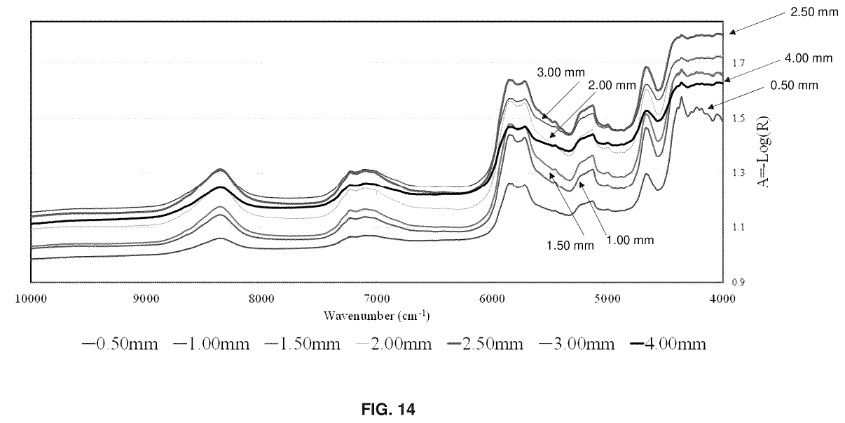

FIG. 10 shows an exemplary gastric residence system, according to some embodiments. The system 1000 comprises a central elastomeric component 1010. The elastomeric component may comprise liquid silicon rubber overmolded onto a polycarbonate component. The shape of the elastomeric component 1010 may be an asterisk or a stellate having six short branches to which six retention members may attach. In some embodiments, the liquid silicon rubber component of the elastomeric component 1010 may be about 5-10 mm, about 6-9 mm, about 7-8 mm, or about 7.5 mm in length at its longest (e.g., from a tip of a first branch to a tip of a second branch directly opposite the first branch). In some embodiments, the polycarbonate component of the elastomeric component 1010 may be about 7-15 mm, about 10-12 mm, or about 11.5 mm in length at its longest. The polycarbonate component may serve as an intercomponent anchor, in that it may extend from the elastomeric component into one or more additional segments, serving to strengthen the connection between the elastomeric component and the retention members. Examples of intercomponent anchors are described in US Patent Application Publication No. 2019/0262265 at, e.g., FIG. 14A and FIG. 14B, FIG. 36A-FIG. 36E, paragraphs [0031]-[0035], and paragraphs [0294]-[0375]. At least one retention member 1020 connected to the elastomeric component 1010 may include a drug eluting component 1026. FIG. 10 shows two such retention members with a drug eluting component. One or more retention members connected to the elastomeric component 1010 may not contain a drug eluting component. FIG. 10 shows five such retention members without drug eluting components. (For the sake of brevity, only one such retention member 1030 is labeled in FIG. 10.)

For a retention member 1020 including a drug eluting component (a drug eluting retention member), a first inactive component 1021 may optionally be connected to a branch of the elastomeric component 1010. The first inactive component 1021 may comprise polycaprolactone (PCL). In some embodiments, the first inactive component 1021 may be injection molded onto the elastomeric component 1010 such that first inactive component 1021 is a part of the elastomeric component 1010, rather than a separate component of the drug eluting retention member 1020.

First inactive component 1021 may be attached to a time-dependent disintegrating matrix component 1022. In some embodiments, time-dependent disintegrating matrix component 1022 may comprise one or more of PCL (e.g., PCL12), poly(ethylene oxide) (e.g., PEO100K), a DL-lactide/glycolide copolymer (e.g., 50/50 DL-Lactide/Glycolide copolymer, acid (pDLG 5002A)), and/or ferrosoferric oxide. Time-dependent disintegrating matrix component 1022 may be configured to degrade in a predictable, time-dependent manner. In some embodiments, degradation of time-dependent disintegrating matrix component 1022 may not be affected by the varying pH of the gastrointestinal system. Exemplary amounts of the components for the time-dependent disintegrating matrix component 1022 are provided in the table below.

| Time-dependent | | | |
|---|---|---|---|
| disintegrating | Formulation 1 | Formulation 2 | Formulation 3 |
| matrix component | (% w/w) | (% w/w) | (% w/w) |
| PCL12 | 40-50 | 43-47 | 44.95 |
| PEO100K | 0.5-3.5 | 1-3 | 2.0 |
| 50/50 DL- | 46-60 | 50-56 | 53.0 |
| Lactide/Glycolide | | | |
| copolymer, acid | | | |
| (pDLG 5002A) | | | |
| Ferrosoferric Oxide | 0.001-1 | 0.01-0.5 | 0.05 |

Time-dependent disintegrating matrix component 1022 may be attached to a second inactive component 1023. The second inactive component 1023 may comprise radiopaque PCL (rPCL), which may comprise PCL (e.g., Corbion PC17) and bismuth subcarbonate. Exemplary amounts of the components for the second inactive component 1023 are provided in the table below.

| Second inactive | Formulation 1 | Formulation 2 | Formulation 3 |
|---|---|---|---|
| component | (% w/w) | (% w/w) | (% w/w) |
| Corbion PC17 | 65-75 | 68-72 | 70 |
| Bismuth | 25-35 | 28-32 | 30 |
| subcarbonate | | | |

Second inactive component 1023 may be attached to an enteric disintegrating matrix component 1024. Enteric disintegrating matrix component 1024 may comprise one or more of PCL, an enteric polymer (e.g., hydroxypropyl methylcellulose acetate succinate MG grade, i.e., HPMCAS-MG), or poloxamer 407 (P407). Enteric disintegrating matrix component 1024 may be designed to break down gradually in a controlled manner during the residence period of the system in the stomach and break down more rapidly if the gastric residence system prematurely passes into the small intestine intact. Enteric disintegrating matrix component 1024 may be relatively resistant to the acidic pH levels of the stomach but may dissolve at the higher pH levels found in the duodenum. Thus, enteric disintegrating matrix component 1024 may protect against undesired passage of an intact gastric residence system into the small intestine. Exemplary amounts of the components for enteric disintegrating matrix component 1024 are provided in the table below.

| Enteric | | | |
|---|---|---|---|
| disintegrating | Formulation 1 | Formulation 2 | Formulation 3 |
| matrix component | (% w/w) | (% w/w) | (% w/w) |
| PCL | 33-50 | 38-45 | 41.5 |
| HPMCAS-MG | 45-68 | 51-62 | 56.5 |
| P407 | 0.5-5 | 1-3 | 2.0 |

Enteric disintegrating matrix component 1024 may be attached to a third inactive component 1025. The third inactive component 1025 may have the same composition as the second inactive component 1023, namely rPCL comprising PCL (e.g., Corbion PC17) and bismuth subcarbonate. Exemplary amounts of the components for third inactive component 1025 are provided in the table below.

| Third inactive | Formulation 1 | Formulation 2 | Formulation 3 |
|---|---|---|---|
| component | (% w/w) | (% w/w) | (% w/w) |
| Corbion PC17 | 65-75 | 68-72 | 70 |
| Bismuth | 25-35 | 28-32 | 30 |
| subcarbonate | | | |

Third inactive component 1025 may be attached to drug eluting component 1026. In some embodiments, drug eluting component 1026 may include risperidone. Risperidone is used as an illustrative example, but it should be understood that any suitable active pharmaceutical ingredient may be used in the gastric residence systems described herein. Drug eluting component 1026 may further include one or more of PCL (e.g., PCL17), copovidone (e.g., Kollidon VA64), poloxamers (e.g., P407), Vitamin E succinate, colloidal silicon dioxide, or colorants (e.g., FD&C Yellow 5 Alum lake, FD&C Blue 1 Alum lake). Exemplary amounts of the components for drug eluting component 1026 are provided in the table below.

| | Formulation 1 | Formulation 2 | Formulation |
|---|---|---|---|
| Drug eluting component | (% w/w) | (% w/w) | 3 (% w/w) |
| Risperidone | 30-40 | 32-38 | 35.0 |
| PCL17 | 50-62 | 53-59 | 55.9 |
| VA64 | 2-8 | 4-6 | 5.0 |
| P407 | 1-5 | 2-4 | 3.0 |
| Vitamin E succinate | 0.05-2 | 0.1-1 | 0.5 |
| Silicon dioxide (SiO₂) | 0.05-2 | 0.1-1 | 0.5 |
| FD&C Yellow 5 Alum | 0.001-1 | 0.01-0.5 | 0.05 |
| lake (16-18%) | | | |
| FD&C Blue 1 Alum lake | 0.001-1 | 0.01-0.5 | 0.05 |
| (11-13%) | | | |

Drug eluting component 1026 may be connected to a fourth inactive component 1027. Optionally, in some embodiments, a portion of a suture 1040 may be embedded in the fourth inactive component 1027, which may connect retention member 1020 (which contains a drug eluting component) to adjacent retention members (e.g., retention member 1030, which does not include a drug eluting component). The fourth inactive component 1027 may comprise PCL (e.g., Corbion PC17). In some embodiments, the fourth inactive component 1027 may further comprise one or more of copovidone (e.g., VA64), poloxamers (e.g., P407), or colorants (e.g., FD&C Blue 1 Alum lake (11-13%)). Exemplary amounts of the components for fourth inactive component 1027 are provided in the table below.

| Fourth inactive | Formulation 1 | Formulation 2 | Formulation 3 |
|---|---|---|---|
| component | (% w/w) | (% w/w) | (% w/w) |
| Corbion PC17 | 60-72 | 63-69 | 66.45 |
| VA64 | 28-36 | 30-34 | 32.00 |
| P407 | 0.5-2.5 | 1-2 | 1.50 |
| FD&C Blue 1 Alum | 0.001-1 | 0.01-0.5 | 0.05 |
| lake (11-13%) | | | |

Approximate radial lengths of the various components on an exemplary drug eluting retention member 1020 are provided in the table below.

| | Radial length | Radial length | Radial length |
|---|---|---|---|
| Component | set 1 | set 2 | set 3 |
| First inactive | 0.1-5 mm | 0.5-3 mm | 1.3 mm |
| component | | | |
| Time-dependent | 0.1-5 mm | 0.5-3 mm | 1.0 mm |
| disintegrating matrix | | | |
| component | | | |
| Second inactive | 0.05-3 mm | 0.1-1.5 mm | 0.5 mm |
| component | | | |
| Enteric | 0.1-5 mm | 0.5-3 mm | 1.85 mm |
| disintegrating | | | |
| matrix component | | | |
| Third inactive | 0.05-3 mm | 0.1-1.5 mm | 0.5 mm |
| component | | | |
| Drug eluting | 3-12 mm | 5-9 mm | 7.3 mm |
| component | | | |
| Fourth inactive | 2-10 mm | 4-7 mm | 5.8 mm |
| component | | | |

In some embodiments, each component of a drug eluting retention member 1020 may have a triangular cross-section. In some embodiments, the triangular cross-section is an equilateral triangular cross section. The base length of the equilateral triangle of each component of drug eluting retention member 1020 is provided in the table below.

| | Cross-section base | Cross-section base | Cross-section |
|---|---|---|---|
| Component | length set 1 | length set 2 | base length set 3 |
| First inactive | 2.5-3.5 mm | 2.9-3.3 mm | 3.3 mm |
| component | | | |
| Time-dependent | 2.5-3.5 mm | 2.9-3.3 mm | 3.3 mm |
| disintegrating matrix | | | |
| component | | | |
| Second inactive | 2.5-3.5 mm | 2.9-3.3 mm | 3.3 mm |
| component | | | |
| Enteric disintegrating | 2.5-3.5 mm | 2.9-3.3 mm | 3.3 mm |
| matrix component | | | |
| Third inactive | 2.5-3.5 mm | 2.9-3.3 mm | 3.3 mm |
| component | | | |
| Drug eluting | 2.5-3.5 mm | 2.9-3.3 mm | 3.3 mm |
| component | | | |
| Fourth inactive | 2.5-3.5 mm | 2.9-3.3 mm | 3.1 mm |
| component | | | |

In some embodiments, drug eluting retention member 1020 may comprise a coating. The coating may comprise one or more of PCL (e.g., PCL17), copovidone (e.g., VA64), or magnesium stearate. In some embodiments, the coating may further comprise trace amounts of ethyl acetate used to dissolve PCL17, VA64, and/or magnesium stearate in preparation for coating. The coating may be applied using a pan coater. In some embodiments, the coat may comprise about 2-4% of the weight of the coated retention member 1020. Exemplary amounts of the components of the coating are provided in the table below.

| | Formulation 1 | Formulation 2 | Formulation 3 |
|---|---|---|---|
| Coating | (% w/w) | (% w/w) | (% w/w) |
| PCL17 | 65-82 | 70-77 | 73.5 |
| VA64 | 20-30 | 22-27 | 24.5 |
| Magnesium stearate | 0.5-5 | 1-3 | 2.0 |

In some embodiments, one retention member of the gastric residence system 1000 comprises a drug eluting retention member 1020. In some embodiments, two retention members of the gastric residence system 1000 comprise a drug eluting retention member 1020. In some embodiments, three retention members of the gastric residence system 1000 comprise a drug eluting retention member 1020. In some embodiments, four retention members of the gastric residence system 1000 comprise a drug eluting retention member 1020. In some embodiments, five retention members of the gastric residence system 1000 comprise a drug eluting retention member 1020. In some embodiments, all six retention members of the gastric residence system 1000 comprise a drug eluting retention member 1020.

In some embodiments, the gastric residence system 1000 comprises one or more retention members 1030 that do not include a drug eluting component (a non-drug eluting retention member). For a non-drug eluting retention member 1030, a first inactive component 1031 may be connected to a branch of the elastomeric component 1010. The first inactive component 1031 may comprise PCL. First inactive component 1031 may have the same composition as first inactive component 1021. Like first inactive component 1021, first inactive component 1031 may be injection molded onto the elastomeric component 1010, such that first inactive component 1031 is a part of the elastomeric component rather than a separate component of a non-drug eluting retention member 1030.

First inactive component 1031 may be attached to a time-dependent disintegrating matrix component 1032. Time-dependent disintegrating matrix component 1032 may have the same composition as time-dependent disintegrating matrix component 1022, namely one or more of PCL (e.g., PCL12), poly(ethylene oxide) (e.g., PEO100K), a DL-lactide/glycolide copolymer (e.g., 50/50 DL-Lactide/Glycolide copolymer, acid (pDLG 5002A)), and/or ferrosoferric oxide.

Time-dependent disintegrating matrix component 1032 may be attached to a second inactive component 1033. Second inactive component 1033 may have the same composition as second inactive component 1023, namely PCL (e.g., Corbion PC17) and bismuth subcarbonate.

Second inactive component 1033 may be attached to an enteric disintegrating matrix component 1034. Enteric disintegrating matrix component 1034 may have the same composition as enteric disintegrating matrix component 1024, namely one or more of PCL, an enteric polymer (e.g., HPMCAS-MG), or a poloxamer (e.g., P407).

Enteric disintegrating matrix component 1034 may be attached to a third inactive component 1035. Third inactive component 1035 may have the same composition as third inactive component 1025, namely PCL (e.g., Corbion PC17) and bismuth subcarbonate.

Third inactive component 1035 may be attached to a fourth inactive component 1036. Fourth inactive component 1036 may have the same composition as fourth inactive component 1027, namely one or more of PCL (e.g., Corbion PC17), copovidone (e.g., VA64), poloxamer (e.g., P407), or colorants (e.g., FD&C Blue 1 Alum lake (11-13%)). Optionally, in some embodiments, a portion of a suture 1040 may be embedded in the fourth inactive component.

Approximate radial lengths of the components on an exemplary non-drug eluting retention member 1030 are provided in the table below.

| | Radial length | Radial length | Radial length |
|---|---|---|---|
| Component | set 1 | set 2 | set 3 |
| First inactive | 0.1-5 mm | 0.5-3 mm | 1.3 mm |
| component | | | |
| Time-dependent | 0.1-5 mm | 0.5-3 mm | 1.0 mm |
| disintegrating matrix | | | |
| component | | | |
| Second inactive | 0.05-3 mm | 0.1-1.5 mm | 0.5 mm |
| component | | | |
| Enteric | 0.1-5 mm | 0.5-3 mm | 1.85 mm |
| disintegrating | | | |
| matrix component | | | |
| Third inactive | 0.05-3 mm | 0.1-1.5 mm | 0.5 mm |
| component | | | |
| Fourth inactive | 5-20 mm | 10-15 mm | 13.1 mm |
| component | | | |

In some embodiments, each component of non-drug eluting retention member 1030 may have a triangular cross-section. In some embodiments, the triangular cross-section is an equilateral triangular cross section. The base length of the equilateral triangle of each segment of non-drug eluting retention member 1030 is provided in the table below.

| | Cross-section base | Cross-section base | Cross-section |
|---|---|---|---|
| Component | length set 1 | length set 2 | base length set 3 |
| First inactive | 2.5-3.5 mm | 2.9-3.3 mm | 3.3 mm |
| component | | | |
| Time-dependent | 2.5-3.5 mm | 2.9-3.3 mm | 3.3 mm |
| disintegrating matrix | | | |
| component | | | |
| Second inactive | 2.5-3.5 mm | 2.9-3.3 mm | 3.3 mm |
| component | | | |
| Enteric disintegrating | 2.5-3.5 mm | 2.9-3.3 mm | 3.3 mm |
| matrix component | | | |
| Third inactive | 2.5-3.5 mm | 2.9-3.3 mm | 3.3 mm |
| component | | | |
| Fourth inactive | 2.5-3.5 mm | 2.9-3.3 mm | 3.1 mm |
| component | | | |

In some embodiments, the diameter of the gastric residence system, at its widest (e.g., from the distal tip of a first retention member to the distal tip of a second retention member opposite the first retention member), is about 40-50 mm, about 42-48 mm, or about 44-46 mm.

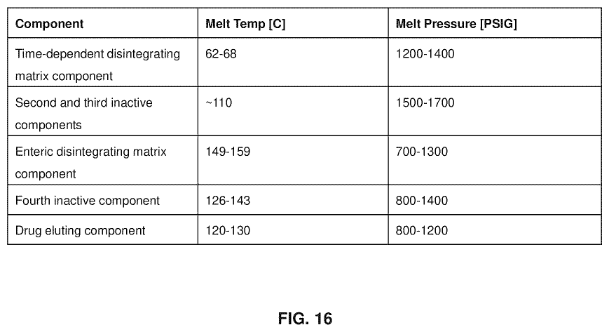

As shown in FIG. 16, a suture 1040 may optionally circumferentially connect adjacent retention members of the gastric residence system. Suture 1040 may help provide the gastric residence system with a more consistent gastric residence time and/or a longer gastric residence time. Gastric residence systems having predictable gastric residence times can minimize the risk of the gastric residence system unfolding too early (e.g., in the esophagus) and causing an obstruction. Gastric residence systems having predictable and/or controllable gastric residence times can also minimize the possibility of the gastric residence system passing through the stomach and unfolding later in the gastrointestinal tract (i.e., intestine), or passing through the gastrointestinal tract without unfolding at all. In some embodiments, the suture may comprise polyglycolic acid (PGA). The suture may have a diameter of about 0.05-1 mm, about 0.1-0.5 mm, or about 0.2 mm. In some embodiments, the suture may comprise a plurality of smaller filaments braided together. The suture 1040 may be embedded into each retention member of the gastric residence system, for example using laser welding or infrared welding techniques.

Gastric residence system 1000 may be folded and enclosed in a sleeve and/or capsule for delivery. In some embodiments, gastric residence system 1000 may first be placed within an inner sleeve before being placed in an outer capsule. The sleeve may comprise hydroxypropyl methylcellulose (HPMC). The sleeve may be a size 0 capsule, cap only. In some embodiments, a capsule may be used in addition to a sleeve. The capsule may comprise HPMC. The capsule may be a Size 00EL capsule. In some embodiments, the capsule may comprise a coating. The capsule coating may comprise one or more of Eudragit E, dibutyl sebacate, or magnesium stearate. The capsule coating may comprise about 35-45 mg per capsule. Exemplary amounts of the components of the capsule coating are provided in the table below.

| | Formulation 1 | Formulation 2 | Formulation 3 |
|---|---|---|---|
| Capsule coating | (% w/w) | (% w/w) | (% w/w) |
| Eudragit E | 75-99 | 85-95 | 90.7 |
| Dibutyl sebacate | 1-10 | 2-6 | 4.64 |
| Magnesium stearate | 1-10 | 2-6 | 4.64 |

### System Dimensions

The retention members of the gastric residence systems and components thereof can have cross-sections in the shape of a circle (in which case the components are cylindrical), a polygon (such as components with a triangular cross-section, rectangular cross-section, or square cross-section), or a pie-shaped cross-section (in which case the components are cylindrical sections). Components with polygon-shaped or pie-shaped cross-sections, and ends of cylindrically-shaped sections which will come into contact with gastric tissue, can have their sharp edges rounded off to provide rounded corners and edges, for enhanced safety in vivo. That is, instead of having a sharp transition between intersecting edges or planes, an arc is used to transition from one edge or plane to another edge or plane. Thus, a “triangular cross-section” includes cross-sections with an approximately triangular shape, such as a triangle with rounded corners. A retention member with a triangular cross-section includes a retention member where the edges are rounded, and the corners at the end of the retention member are rounded. Rounded corners and edges are also referred to as fillet corners, filleted corners, fillet edges, or filleted edges.

In some embodiments, a stellate-shaped gastric residence system is about 30 mm to about 60 mm when unfolded (retention member extended). In some embodiments, the stellate system is about 41 mm to about 51 mm when unfolded. In some embodiments, the stellate system is about 45 mm to about 47 mm when unfolded. In some embodiments, the stellate system is about 46 mm when unfolded.

Once the gastric residence system is assembled, the system must be able to adopt a compacted state with dimensions that enable the patient to swallow the system (or for the system to be introduced into the stomach by alternate means, such as a feeding tube or gastrostomy tube). Typically, the system is held in the compacted state by a container such as a capsule. Upon entry into the stomach, the system is then released from the container and adopts an uncompacted state, that is, an expanded conformation, with dimensions that prevent passage of the system through the pyloric sphincter, thus permitting retention of the system in the stomach.

Accordingly, the system should be capable of being placed inside a standard-sized capsule of the type commonly used in pharmacy. Standard capsule sizes in use in the United States are provided below in the Capsule Table below (see “Draft Guidance for Industry on Size, Shape, and Other Physical Attributes of Generic Tablets and Capsules” at URL www.regulations.gov/#!documentDetail; D=FDA-2013-N-1434-0002). As these are the outer dimensions of the capsule, and as dimensions will vary slightly between capsule manufacturers, the system should be capable of adopting a configuration which is about 0.5 to about 1 mm smaller than the outer diameter shown, and about 1 mm to about 2 mm shorter than the length shown in the Capsule Table.

Capsule Table

| Capsule Size | Outer Diameter (mm) | Length (mm) |
|---|---|---|
| 000 | 9.9 | 26.1 |
| 00 | 8.5 | 23.3 |
| 0 | 7.6 | 21.7 |
| 1 | 6.9 | 19.4 |
| 2 | 6.3 | 18.0 |
| 3 | 5.8 | 15.9 |
| 4 | 5.3 | 14.3 |
| 5 | 4.9 | 11.1 |

Capsules can be made of materials well-known in the art, such as gelatin or hydroxypropyl methylcellulose. In one embodiment, the capsule is made of a material that dissolves in the gastric environment, but not in the oral or esophageal environment, which prevents premature release of the system prior to reaching the stomach.

In one embodiment, the system will be folded or compressed into a compacted state in order to fit into the capsule, for example, in a manner such as that shown in FIG. 1B. Once the capsule dissolves in the stomach, the system will adopt a configuration suitable for gastric retention, for example, in a manner such as that shown in FIG. 1A. Preferred capsule sizes are 00 and 00e1 (a 00e1-size capsule has the approximate length of a 000 capsule and the approximate width of a 00 capsule), which then places constraints on the length and diameter of the folded system.

Once released from the container, the system adopts an uncompacted state with dimensions suitable to prevent passage of the gastric residence system through the pyloric sphincter. In one embodiment, the system has at least two perpendicular dimensions, each of at least about 2 cm in length; that is, the gastric residence system measures at least about 2 cm in length over at least two perpendicular directions. In another embodiment, the perimeter of the system in its uncompacted state, when projected onto a plane, has two perpendicular dimensions, each of at least about 2 cm in length. The two perpendicular dimensions can independently have lengths of from about 2 cm to about 7 cm, about 2 cm to about 6 cm, about 2 cm to about 5 cm, about 2 cm to about 4 cm, about 2 cm to about 3 cm, about 3 cm to about 7 cm, about 3 cm to about 6 cm, about 3 cm to about 5 cm, about 3 cm to about 4 cm, about 4 cm to about 7 cm, about 4 cm to about 6 cm, about 4 cm to about 5 cm, or about 4 cm to about 4 cm. These dimensions prevent passage of the gastric residence system through the pyloric sphincter. For star-shaped polymers with N retention members (where N is greater than or equal to three, such as N=6), the retention members can have dimensions such that the system has at least two perpendicular dimensions, each of length as noted above. These two perpendicular dimensions are chosen as noted above in order to promote retention of the gastric residence system.

The system is designed to eventually break apart in the stomach at the end of the desired residence time (residence period), at which point the remaining components of the system are of dimensions that permit passage of the system through the pyloric sphincter, small intestine, and large intestine. Finally, the system is eliminated from the body by defecation, or by eventual complete dissolution of the system in the small and large intestines. Thus, laser linker components or disintegrating matrices are placed in the gastric residence systems of the invention in a configuration such that, at the end of the desired residence period when the laser linker components or disintegrating matrices break or dissolve, the uncoupled components of the gastric residence system have dimensions suitable for passage through the pyloric sphincter and elimination from the digestive tract.

### Residence Time

The residence time of the gastric residence system is defined as the time between administration of the system to the stomach and exit of the system from the stomach. In one embodiment, the gastric residence system has a residence time of about 24 hours, or up to about 24 hours. In one embodiment, the gastric residence system has a residence time of about 48 hours, or up to about 48 hours. In one embodiment, the gastric residence system has a residence time of about 72 hours, or up to about 72 hours. In one embodiment, the gastric residence system has a residence time of about 96 hours, or up to about 96 hours. In one embodiment, the gastric residence system has a residence time of about 5 days, or up to about 5 days. In one embodiment, the gastric residence system has a residence time of about 6 days, or up to about 6 days. In one embodiment, the gastric residence system has a residence time of about 7 days (about one week), or up to about 7 days (about one week). In one embodiment, the gastric residence system has a residence time of about 10 days, or up to about 10 days. In one embodiment, the gastric residence system has a residence time of about 14 days (about two weeks), or up to about 14 days (about two weeks).

In one embodiment, the gastric residence system has a residence time between about 24 hours and about 7 days. In one embodiment, the gastric residence system has a residence time between about 48 hours and about 7 days. In one embodiment, the gastric residence system has a residence time between about 72 hours and about 7 days. In one embodiment, the gastric residence system has a residence time between about 96 hours and about 7 days. In one embodiment, the gastric residence system has a residence time between about 5 days and about 7 days. In one embodiment, the gastric residence system has a residence time between about 6 days and about 7 days.

In one embodiment, the gastric residence system has a residence time between about 24 hours and about 10 days. In one embodiment, the gastric residence system has a residence time between about 48 hours and about 10 days. In one embodiment, the gastric residence system has a residence time between about 72 hours and about 10 days. In one embodiment, the gastric residence system has a residence time between about 96 hours and about 10 days. In one embodiment, the gastric residence system has a residence time between about 5 days and about 10 days. In one embodiment, the gastric residence system has a residence time between about 6 days and about 10 days. In one embodiment, the gastric residence system has a residence time between about 7 days and about 10 days.

In one embodiment, the gastric residence system has a residence time between about 24 hours and about 14 days. In one embodiment, the gastric residence system has a residence time between about 48 hours and about 14 days. In one embodiment, the gastric residence system has a residence time between about 72 hours and about 14 days. In one embodiment, the gastric residence system has a residence time between about 96 hours and about 14 days. In one embodiment, the gastric residence system has a residence time between about 5 days and about 14 days. In one embodiment, the gastric residence system has a residence time between about 6 days and about 14 days. In one embodiment, the gastric residence system has a residence time between about 7 days and about 14 days. In one embodiment, the gastric residence system has a residence time between about 10 days and about 14 days.

The gastric residence system releases a therapeutically effective amount of agent during at least a portion of the residence time or residence period during which the system resides in the stomach. In one embodiment, the system releases a therapeutically effective amount of agent during at least about 25% of the residence time. In one embodiment, the system releases a therapeutically effective amount of agent during at least about 50% of the residence time. In one embodiment, the system releases a therapeutically effective amount of agent during at least about 60% of the residence time. In one embodiment, the system releases a therapeutically effective amount of agent during at least about 70% of the residence time. In one embodiment, the system releases a therapeutically effective amount of agent during at least about 75% of the residence time. In one embodiment, the system releases a therapeutically effective amount of agent during at least about 80% of the residence time. In one embodiment, the system releases a therapeutically effective amount of agent during at least about 85% of the residence time. In one embodiment, the system releases a therapeutically effective amount of agent during at least about 90% of the residence time. In one embodiment, the system releases a therapeutically effective amount of agent during at least about 95% of the residence time. In one embodiment, the system releases a therapeutically effective amount of agent during at least about 98% of the residence time. In one embodiment, the system releases a therapeutically effective amount of agent during at least about 99% of the residence time.

### Evaluation of Release Characteristics

The release characteristics of agent from components, retention members, and gastric residence systems can be evaluated by various assays. Release of agent in vitro from components, retention members, and gastric residence systems can be measured by immersing a component, retention member, or gastric residence system in a liquid, such as water, 0.1N HCl, fasted state simulated gastric fluid (FaSSGF), or fed state simulated gastric fluid (FeSSGF). In one embodiment, fasted state simulated gastric fluid (FaSSGF) is used for release assays. Simulated gastric fluid indicates either fasted state simulated gastric fluid (FaSSGF) or fed state simulated gastric fluid (FeSSGF); when a limitation is specified as being measured in simulated gastric fluid (SGF), the limitation is met if the limitation holds in either fasted state simulated gastric fluid (FaSSGF) or fed state simulated gastric fluid (FeSSGF). For example, if a component of a retention member is indicated as releasing at least 10% of an agent over the first 24 hours in simulated gastric fluid, the limitation is met if the component releases at least 10% of the agent over the first 24 hours in fasted state simulated gastric fluid, or if the component releases at least 10% of the agent over the first 24 hours in fed state simulated gastric fluid.

Ethanol burst release is typically measured by immersing a component, retention member, or gastric residence system in a solution of 40% ethanol and 60% fasted state simulated gastric fluid for one hour, followed by immersing the same component, retention member, or gastric residence system in 100% fasted state simulated gastric fluid for the remainder of the test period, and measuring release of agent at appropriate time points. This test is designed to simulate the effects of consumption of alcoholic beverages by a patient having a gastric residence system of the invention deployed in the patient's stomach.

While in vitro tests can be performed using components, retention members, or gastric residence systems, use of individual components for in vitro tests is most convenient for rapid evaluation of the release characteristics. When in vitro tests are done to compare release rates under different conditions (such as release in 100% FaSSGF versus release in 40% ethanol/60% FaSSGF), the comparison solutions are kept at the same temperature, such as room temperature, 25° C., or 37° C.

In vivo tests can be performed in animals such as dogs (for example, beagle dogs or hound dogs) and swine. For in vivo tests, a gastric residence system is used, since an individual segment or retention member would not be retained in the stomach of the animal. Blood samples can be obtained at appropriate time points, and, if desired, gastric contents can be sampled by cannula or other technique.

Clinical trials in humans, conducted in accordance with appropriate laws, regulations, and institutional guidelines, also provide in vivo data.

### Gastric Delivery Pharmacokinetics for Gastric Residence Systems

The gastric residence systems of the invention provide for high bioavailability of the agent as measured by AUC_{inf} after administration of the systems, relative to the bioavailability of a conventional oral formulation of the agent. The systems also provide for maintenance of an approximately constant plasma level or a substantially constant plasma level of the agent.

Relative bioavailability, FREL, of two different formulations, formulation A and formulation B, is defined as:

$${F}_{REL} = 100 \times ( A U {C}_{A} \times {Dose}_{B} ) / ( A U {C}_{B} \times {Dose}_{A} )$$

where AUC_{A} is the area under the curve for formulation A, AUC_{B} is the area under the curve for formulation B, Dose_{A} is the dosage of formulation A used, and Dose_{B} is the dosage of formulation B used. AUC, the area under the curve for the plot of agent plasma concentration versus time, is usually measured at the same time (t) after administration of each formulation, in order to provide the relative bioavailability of the formulations at the same time point. AUC_{inf} refers to the AUC measured or calculated over “infinite” time, that is, over a period of time starting with initial administration, and ending where the plasma level of the agent has dropped to a negligible amount.

In one embodiment, the substantially constant plasma level of agent provided by the gastric residence systems of the invention can range from at or above the trough level of the plasma level of agent when administered daily in a conventional oral formulation (that is, Cₘᵢₙ of agent administered daily in immediate-release formulation) to at or below the peak plasma level of agent when administered daily in a conventional oral formulation (that is, Cₘₐₓ of agent administered daily in immediate-release formulation). In some embodiments, the substantially constant plasma level of agent provided by the gastric residence systems of the invention can be about 50% to about 90% of the peak plasma level of agent when administered daily in a conventional oral formulation (that is, Cₘₐₓ of agent administered daily in immediate-release formulation). The substantially constant plasma level of agent provided by the gastric residence systems of the invention can be about 75% to about 125% of the average plasma level of agent when administered daily in a conventional oral formulation (that is, Cave of agent administered daily in immediate-release formulation). The substantially constant plasma level of agent provided by the gastric residence systems of the invention can be at or above the trough level of plasma level of agent when administered daily in a conventional oral formulation (that is, Cₘᵢₙ of agent administered daily in immediate-release formulation), such as about 100% to about 150% of Cₘᵢₙ.

The gastric residence systems of the invention can provide bioavailability of agent released from the system of at least about 50%, at least about 60%, at least about 70%, or at least about 80% of that provided by an immediate release form comprising the same amount of agent. As indicated above, the bioavailability is measured by the area under the plasma concentration-time curve (AUC_{inf}).

### Dissolution Profile, Bioavailability and Pharmacokinetics for Gastric Residence Systems

The gastric residence systems described herein provide a steady release of an agent or a pharmaceutically acceptable salt thereof over an extended period of time. The systems are designed to release a therapeutically effective amount of an agent over the period of residence in the stomach. The release of agent can be measured in vitro or in vivo to establish the dissolution profile (elution profile, release rate) of the agent from a given residence system in a specific environment. The dissolution profile can be specified as a percentage of the original amount of agent present in the system which elutes from the system over a given time period.

Thus, in some embodiments, the agent contained in a gastric residence system can have a dissolution profile of 10-20% release between zero hours and 24 hours in a given environment. That is, over the 24-hour period after initial introduction of the gastric residence system into the environment of interest, 10-20% of the initial agent contained in the system elutes from the system.

The environment of interest can be 1) the stomach of a patient (that is, an in vivo environment), or 2) simulated gastric fluid (that is, an in vitro environment).

The gastric residence systems of the invention provide for high bioavailability of the agent as measured by AUC_{inf} after administration of the systems, relative to the bioavailability of a conventional oral formulation of the agent. The systems also provide for maintenance of a substantially constant plasma level of the agent.

Parameters of interest for release include the linearity of release over the residence period of the gastric residence systems, the standard deviation of release over the residence period (which is related to linearity of release; a standard deviation of zero indicates that release is linear over the entire residence period), the release over the initial six hours of residence (that is, burst release upon initial administration), and total release of agent over the residence period. In one embodiment, the residence period is seven days, although other periods, such as two, three, four, five, six, eight, nine, ten, 11, 12, 13, or 14 days can be used.

Linearity of agent release over the residence period refers to the amount released during each 24-hour period of residence. For a seven-day period of residence, it is desirable that about the amount of agent is released each day, i.e., that linearity of agent release is maximized. This will minimize the standard deviation of daily agent release over the residence period. In some embodiments, the gastric release systems have a variation (or a standard deviation) for daily agent release of less than about 100%, less than about 90%, less than about 80%, less than about 70%, less than about 60%, less than about 50%, less than about 40%, less than about 30%, less than about 25%, less than about 20%, less than about 15%, less than about 10%, or less than about 5%, over the period of residence. In some embodiments, the period of residence can be about three days, about seven days, about ten days, or about two weeks.

Minimization of burst release, that is, release over the initial period of residence (such as six hours, twelve hours, or 24 hours after administration of a gastric residence system) is desirable in order to maintain a predictable and steady release profile. If T is the total agent release over the residence period (in units of mass), and D is the number of days of the residence period, then completely linear release would mean that about T/D mass of agent is released per day. If the period over which burst release is measured is the first six hours, then a linear release profile will result in 0.25×T/D mass of agent released during the first six hours. In percentage terms of the total amount of agent released over the residence period of D days, linear release would be about 100/D % of agent per day, and a linear release over the first six hours would be 25/D %. (Note that 100% in this context indicates the total amount of agent released, regardless of how much agent is contained in the initial formulation.) Thus, for a seven day residence period, linear release over the first six hours would be about 3.6% of the total amount of agent released over the seven-day period.

In some embodiments, during the initial six hours of residence after administration the gastric residence systems release about 0.2 to about 2 times T/D of the total mass of agent T released over the residence period of D days, or about 0.2 to about 1.75 times T/D of the total mass of agent T released over the residence period of D days, or about 0.2 to about 1.5 times T/D of the total mass of agent T released over the residence period of D days, or about 0.2 to about 1.25 times T/D of the total mass of agent T released over the residence period of D days, or about 0.2 to about 1 times T/D of the total mass of agent T released over the residence period of D days, or about 0.2 to about 0.8 times T/D of the total mass of agent T released over the residence period of D days, or about 0.2 to about 0.75 times T/D, or about 0.2 to about 0.7 times T/D, or about 0.2 to about 0.6 times T/D, or about 0.2 to about 0.5 times T/D, or about 0.2 to about 0.4 times T/D, or about 0.2 to about 0.3 times T/D, or about 0.25 to about 2 times T/D, or about 0.3 to about 2 times T/D, or about 0.4 to about 2 times T/D, or about 0.5 to about 2 times T/D, or about 0.6 to about 2 times T/D, or about 0.7 to about 2 times T/D, or about 0.25 to about 1.5 times T/D, or about 0.3 to about 1.5 times T/D, or about 0.4 to about 1.5 times T/D, or about 0.5 to about 1.5 times T/D, or about 0.6 to about 1.5 times T/D, or about 0.7 to about 1.5 times T/D, or about 0.25 to about 1.25 times T/D, or about 0.3 to about 1.25 times T/D, or about 0.4 to about 1.25 times T/D, or about 0.5 to about 1.25 times T/D, or about 0.6 to about 1.25 times T/D, or about 0.7 to about 1.25 times T/D, or about 0.25 to about 1 times T/D, or about 0.3 to about 1 times T/D, or about 0.4 to about 1 times T/D, or about 0.5 to about 1 times T/D, or about 0.6 to about 1 times T/D, or about 0.7 to about 1 times T/D, or about 0.25 times T/D, or about 0.25 to about 0.8 times T/D, or about 0.3 to about 0.8 times T/D, or about 0.4 to about 0.8 times T/D, or about 0.5 to about 0.8 times T/D, or about 0.6 to about 0.8 times T/D, or about 0.7 to about 0.8 times T/D, or about 0.8 times T/D, about 1 times T/D, about 1.25 times T/D, about 1.5 times T/D, or about 2 times T/D.

In some embodiment of the gastric residence systems, during the initial six hours of residence after administration the gastric residence systems release about 2% to about 10% of the total mass of agent released over the residence period, or about 3% to about 10%, or about 4% to about 10%, or about 5% to about 10%, or about 6% to about 10%, or about 7% to about 10%, or about 8% to about 10%, or about 9% to about 10%, or about 2% to about 9%, or about 2% to about 8%, or about 2% to about 7%, or about 2% to about 6%, or about 2% to about 5%, or about 2% to about 4%, or about 2% to about 3%.

In some embodiments of the gastric residence systems, where the gastric residence systems have a residence period of about seven days, during the initial six hours of residence after administration the gastric residence systems release about 2% to about 10% of the total mass of agent released over the residence period of seven days, or about 3% to about 10%, or about 4% to about 10%, or about 5% to about 10%, or about 6% to about 10%, or about 7% to about 10%, or about 8% to about 10%, or about 9% to about 10%, or about 2% to about 9%, or about 2% to about 8%, or about 2% to about 7%, or about 2% to about 6%, or about 2% to about 5%, or about 2% to about 4%, or about 2% to about 3%.

In some embodiments, during the initial 24 hours of residence after administration, the gastric residence systems release about 10% to about 35% of the total mass of agent released over the residence period, or about 10% to about 30%, or about 10% to about 25%, or about 10% to about 20%, or about 10% to about 15%, or about 15% to about 35%, or about 15% to about 35%, or about 15% to about 30%, or about 20% to about 30%, or about 25% to about 35%, or about 25% to about 30%, or about 30% to about 35%.

In some embodiments, where the gastric residence systems have a residence period of about seven days, during the initial 24 hours of residence after administration the gastric residence systems release about 10% to about 35% of the total mass of agent released over the residence period of seven days, or about 10% to about 30%, or about 10% to about 25%, or about 10% to about 20%, or about 10% to about 15%, or about 15% to about 35%, or about 15% to about 35%, or about 15% to about 30%, or about 20% to about 30%, or about 25% to about 35%, or about 25% to about 30%, or about 30% to about 35%.

### Methods of Manufacture: Manufacture of Gastric Residence Systems

The gastric residence system or components thereof (which may thereafter be laser welded together to produce a gastric residence system) can be produced using three-dimensional printing techniques. Three-dimensional printing of components of the gastric residence system, such as retention members or retention member components, is performed using commercially-available equipment. Three-dimensional printing has been used for pharmaceutical preparation; see Khaled et al., “Desktop 3D printing of controlled release pharmaceutical bilayer tablets,” International Journal of Pharmaceutics 461:105-111 (2014); U.S. Pat. No. 7,276,252; Alhnan et al., “Emergence of 3D Printed Dosage Forms: Opportunities and Challenges,” Pharm. Res., May 18, 2016, PubMed PMID: 27194002); Yu et al., “Three-dimensional printing in pharmaceutics: promises and problems,” J. Pharm. Sci. 97 (9): 3666-3690 (2008); and Ursan et al., “Three-dimensional drug printing: A structured review,” J. Am. Pharm. Assoc. 53 (2): 136-44 (2013).

The initial feedstocks for three-dimensional printing are polymers or polymer blends (e.g. enteric polymers, time-dependent polymers, or blends of one or more of an agent, an agent salt, a drug, an excipient, etc., with a carrier polymer, enteric polymers, or time-dependent polymers). The polymer or ingredients which are to be used for one region of the component or retention member to be manufactured are mixed and pelletized using hot melt extrusion. The polymer or blended polymer material is extruded through a circular die, creating a cylindrical fiber which is wound around a spool.

Multiple spools are fed into the 3D printer (such as a Hyrel Printer, available from Hyrel 3D, Norcross, Ga., United States), to be fed into their representative print heads. The print heads heat up and melt the material at the nozzle, and lay down a thin layer of material (polymer or polymer blend) in a specific position on the piece being manufactured. The material cools and hardens within seconds, and the next layer is added until the complete structure is formed. The quality of the gastric residence system is dependent on the feed rate, nozzle temperature, and printer resolution; feed rate and nozzle temperature can be adjusted to obtain the desired quality.

Three-dimensional printing can be used to manufacture individual retention members, or components of retention members. Three-dimensional printing can also be used to prepare a bulk configuration, such as a consolidated “slab,” similar to that prepared by co-extrusion methods described herein. The bulk configuration can be cut into individual pieces (that is, individual retention members or individual components) as needed.

In some embodiments of the invention, producing an entire retention member of the gastric residence system by three-dimensional printing of the retention member is contemplated. In some embodiments of the invention, producing an individual component of a retention member of the gastric residence system by three-dimensional printing of the component of a retention member is contemplated. In some embodiments, a retention member or a segment thereof is produced by three-dimensional printing of adjacent components in a bulk configuration, such as a slab configuration. The three-dimensional printing can be followed by cutting the bulk configuration into pieces which have the desired shape of the retention member or component thereof. The three-dimensional printing can be followed by compression molding of portions of the bulk configuration into pieces which have the desired shape of the retention member or component thereof.

Three-dimensional printing is often accomplished by feeding a rod or fiber of a solid material to a print head, where it is melted and deposited with subsequent solidification, in a technique known as fused deposition modeling (sometimes also called extrusion deposition); see U.S. Pat. Nos. 5,121,329 and 5,340,433. The methods described herein for the manufacture of carrier polymer-drug components can also be used to manufacture feed material, which can be used in the manufacture via three-dimensional printing of components of the gastric residence systems.

Components of the gastric residence systems can alternatively be manufactured by co-extrusion. Most of the various configurations for the segments discussed herein can be made by either three-dimensional printing or co-extrusion. However, co-extrusion is less expensive, and can be run as a continuous process, as opposed to three-dimensional printing, which is generally run as a batch process.

Co-extrusion of components of the gastric residence system, such as a retention member, or a component of a retention member, can be performed using commercially-available equipment, combined with customized co-extruder plumbing and customized dies for the desired configuration. The initial feedstocks for co-extrusion are polymers or polymer blends (e.g., enteric polymers, time-dependent polymers, or blends of one or more of an agent, an agent salt, a drug, an excipient, etc., with a carrier polymer, enteric polymers, or time-dependent polymers). The polymer or ingredients which are to be used for one region of the component or retention member to be manufactured are mixed and pelletized using hot melt extrusion. The polymer pellets thus formed are placed into hoppers above single screw extruders and dried to remove surface moisture. Pellets are gravimetrically fed into individual single-screw extruders, where they are melted and pressurized for co-extrusion.

The appropriate molten polymers are then pumped through custom designed dies with multiple channels where they form the required geometry. The composite polymer block is cooled (water-cooled, air-cooled, or both) and cut or stamped into the desired shape, including, but not limited to, such shapes as triangular prisms, rectangular prisms, or cylinder sections (pie-shaped wedges).

In some embodiments of the invention, producing an entire retention member of the gastric residence system by co-extruding the retention member is contemplated. In some embodiments of the invention, producing an individual component of a retention member of the gastric residence system by co-extruding the component of a retention member is contemplated. In some embodiments, a retention member or a component thereof is produced by co-extruding adjacent components in a bulk configuration, such as a slab configuration. The co-extruding can be followed by cutting the bulk configuration into pieces which have the desired shape of the retention member or component thereof. The co-extruding can be followed by compression molding of portions of the bulk configuration into pieces which have the desired shape of the retention member or component thereof.

In some embodiments, a retention member or a component thereof is produced by co-extruding adjacent components in a bulk configuration, such as a slab configuration, while also co-extruding an additional polymer or polymers within the components. The co-extruding the additional polymer or polymers within the components can be performed in an islands-in-the-sea configuration. The co-extruding can be followed by cutting the bulk configuration into pieces which have the desired shape of the retention member or component thereof. The co-extruding can be followed by compression molding of portions of the bulk configuration into pieces which have the desired shape of the retention member or component thereof.

Once the various components of the gastric residence system are extruded or otherwise manufactured, the components may be attached, for example using the laser welding techniques described herein.

### Agent Particle Size and Milling

Control of particle size used in the gastric residence systems is important for both optimal release of agent and mechanical stability of the systems. The particle size of the agents affects the surface area of the agents available for dissolution when gastric fluid permeates the carrier polymer-agent segments of the system. Also, as the retention members of the systems are relatively thin in diameter (for example, 1 millimeter to 5 millimeters), the presence of a particle of agent of a size in excess of a few percent of the diameter of the retention members will result in a weaker retention member, both before the agent elutes from the device, and after elution when a void is left in the space formerly occupied by the agent particle. Such weakening of the retention members is disadvantageous, as it may lead to premature breakage and passage of the system before the end of the desired residence period.

In one embodiment, the agent particles used for blending into the carrier polymer-agent components are smaller than about 100 microns in diameter. In another embodiment, the agent particles are smaller than about 75 microns in diameter. In another embodiment, the agent particles are smaller than about 50 microns in diameter. In another embodiment, the agent particles are smaller than about 40 microns in diameter. In another embodiment, the agent particles are smaller than about 30 microns in diameter. In another embodiment, the agent particles are smaller than about 25 microns in diameter. In another embodiment, the agent particles are smaller than about 20 microns in diameter. In another embodiment, the agent particles are smaller than about 10 microns in diameter. In another embodiment, the agent particles are smaller than about 5 microns in diameter.

In one embodiment, at least about 80% of the agent particles used for blending into the carrier polymer-agent components are smaller than about 100 microns in diameter. In another embodiment, at least about 80% of the agent particles are smaller than about 75 microns in diameter. In another embodiment, at least about 80% of the agent particles are smaller than about 50 microns in diameter. In another embodiment, at least about 80% of the agent particles are smaller than about 40 microns in diameter. In another embodiment, at least about 80% of the agent particles are smaller than about 30 microns in diameter. In another embodiment, at least about 80% of the agent particles are smaller than about 25 microns in diameter. In another embodiment, at least about 80% of the agent particles are smaller than about 20 microns in diameter. In another embodiment, at least about 80% of the agent particles are smaller than about 10 microns in diameter. In another embodiment, at least about 80% of the agent particles are smaller than about 5 microns in diameter.

In one embodiment, at least about 80% of the mass of the agent particles used for blending into the carrier polymer-agent components have sizes between about 1 micron and about 100 microns in diameter. In another embodiment, at least about 80% of the mass of the agent particles have sizes between about 1 micron and about 75 microns in diameter. In another embodiment, at least about 80% of the mass of the agent particles have sizes between about 1 micron and about 50 microns in diameter. In another embodiment, at least about 80% of the mass of the agent particles have sizes between about 1 micron and about 40 microns in diameter. In another embodiment, at least about 80% of the mass of the agent particles have sizes between about 1 micron and about 30 microns in diameter. In another embodiment, at least about 80% of the mass of the agent particles have sizes between about 1 micron and about 25 microns in diameter. In another embodiment, at least about 80% of the mass of the agent particles have sizes between about 1 micron and about 20 microns in diameter. In another embodiment, at least about 80% of the mass of the agent particles have sizes between about 1 micron and about 10 microns in diameter. In another embodiment, at least about 80% of the mass of the agent particles have sizes between about 1 micron and about 5 microns in diameter.

In one embodiment, at least about 80% of the mass of the agent particles used for blending into the carrier polymer-agent components have sizes between about 2 microns and about 100 microns in diameter. In another embodiment, at least about 80% of the mass of the agent particles have sizes between about 2 microns and about 75 microns in diameter. In another embodiment, at least about 80% of the mass of the agent particles have sizes between about 2 microns and about 50 microns in diameter. In another embodiment, at least about 80% of the mass of the agent particles have sizes between about 2 microns and about 40 microns in diameter. In another embodiment, at least about 80% of the mass of the agent particles have sizes between about 2 microns and about 30 microns in diameter. In another embodiment, at least about 80% of the mass of the agent particles have sizes between about 2 microns and about 25 microns in diameter. In another embodiment, at least about 80% of the mass of the agent particles have sizes between about 2 microns and about 20 microns in diameter. In another embodiment, at least about 80% of the mass of the agent particles have sizes between about 2 microns and about 10 microns in diameter. In another embodiment, at least about 80% of the mass of the agent particles have sizes between about 2 microns and about 5 microns in diameter.

In one embodiment, at least about 80% of the mass of the agent particles used for blending into the carrier polymer-agent components have sizes between about 5 microns and about 100 microns in diameter. In another embodiment, at least about 80% of the mass of the agent particles have sizes between about 5 microns and about 75 microns in diameter. In another embodiment, at least about 80% of the mass of the agent particles have sizes between about 5 microns and about 50 microns in diameter. In another embodiment, at least about 80% of the mass of the agent particles have sizes between about 5 microns and about 40 microns in diameter. In another embodiment, at least about 80% of the mass of the agent particles have sizes between about 5 microns and about 30 microns in diameter. In another embodiment, at least about 80% of the mass of the agent particles have sizes between about 5 microns and about 25 microns in diameter. In another embodiment, at least about 80% of the mass of the agent particles have sizes between about 5 microns and about 20 microns in diameter. In another embodiment, at least about 80% of the mass of the agent particles have sizes between about 5 microns and about 10 microns in diameter.

The particle size of the agents can be readily adjusted by milling. Several milling techniques are available to reduce larger particles to smaller particles of desired size. Fluid energy milling is a dry milling technique which uses inter-particle collisions to reduce the size of particles. A type of fluid energy mill called an air jet mill shoots air into a cylindrical chamber in a manner so as to maximize collision between agent particles. Ball milling utilizes a rolling cylindrical chamber which rotates around its principal axis. The agent and grinding material (such as steel balls, made from chrome steel or CR-NI steel; ceramic balls, such as zirconia; or plastic polyamides) collide, causing reduction in particle size of the agent. Ball milling can be performed in either the dry state, or with liquid added to the cylinder where the agent and the grinding material are insoluble in the liquid. Further information regarding milling is described in the chapter by R. W. Lee et al. entitled “Particle Size Reduction” in Water-Insoluble Drug Formulation, Second Edition (Ron Liu, editor), Boca Raton, Fla.: CRC Press, 2008; and in the chapter by A. W. Brzeczko et al. entitled “Granulation of Poorly Water-Soluble Drugs” in Handbook of Pharmaceutical Granulation Technology, Third Edition (Dilip M. Parikh, editor), Boca Raton, Fla.: CRC Press/Taylor & Francis Group, 2010 (and other sections of that handbook). Fluid energy milling (i.e., air jet milling) is a useful method of milling, as it is more amenable to scale-up compared to other dry milling techniques such as ball milling.

Substances can be added to the agent material during milling to assist in obtaining particles of the desired size, and minimize aggregation during handling. Silica (silicon dioxide, SiO2) is a useful milling additive, as it is inexpensive, widely available, and non-toxic. Other additives which can be used include silica, calcium phosphate, powdered cellulose, colloidal silicon dioxide, hydrophobic colloidal silica, magnesium oxide, magnesium silicate, magnesium trisilicate, talc, polyvinylpyrrolidone, cellulose ethers, polyethylene glycol, polyvinyl alcohol, and surfactants. In particular, hydrophobic particles less than 5 microns in diameter are particularly prone to agglomeration, and hydrophilic additives are used when milling such particles. A weight/weight ratio of about 0.1% to about 5% of milling additive, such as silica, can be used for fluid milling or ball milling, or about 0.1% to about 4%, about 0.1% to about 3%, about 0.1% to about 2%, about 0.1% to about 1%, about 1% to about 5%, about 1% to about 4%, about 1% to about 3%, about 1% to about 2%, or about 0.1%, about 0.5%, about 1%, about 2%, about 3%, about 4% or about 5%.

After milling, particles can be passed through meshes of appropriate size to obtain particles of the desired size. To obtain particles of a desired maximum size, particles are passed through a mesh with holes of the maximum size desired; particles which are too large will be retained on the mesh, and particles which pass through the mesh will have the desired maximum size. To obtain particles of a desired minimum size, particles are passed through a mesh with holes of the minimum size desired; particles which pass through the mesh are too small, and the desired particles will be retained on the mesh.

### Methods of Manufacture of Drug Eluting Components

Blending temperatures for incorporation of the agent into polymeric matrices typically range from about 80° C. to about 120° C., although higher or lower temperatures can be used for polymers which are best blended at temperatures outside that range. When agent particles of a particular size are used, and it is desired that the size of the particles be maintained during and after blending, blending can be done at temperatures below the melting point of the agent, so as to maintain the desired size of the particles. Otherwise, temperatures can be used which melt both the polymer and the agent. Blending temperatures should be below the degradation temperature of the agent. In one embodiment, less than about 2% of the agent is degraded during manufacture. In one embodiment, less than about 1.5% of the agent is degraded during manufacture. In one embodiment, less than about 1% of the agent is degraded during manufacture. In one embodiment, less than about 0.75% of the agent is degraded during manufacture. In one embodiment, less than about 0.5% of the agent is degraded during manufacture. In one embodiment, less than about 0.4% of the agent is degraded during manufacture. In one embodiment, less than about 0.3% of the agent is degraded during manufacture. In one embodiment, less than about 0.2% of the agent is degraded during manufacture. In one embodiment, less than about 0.15% of the agent is degraded during manufacture. In one embodiment, less than about 0.1% of the agent is degraded during manufacture. In one embodiment, less than about 0.05% of the agent is degraded during manufacture. In one embodiment, less than about 0.04% of the agent is degraded during manufacture. In one embodiment, less than about 0.03% of the agent is degraded during manufacture. In one embodiment, less than about 0.02% of the agent is degraded during manufacture. In one embodiment, less than about 0.01% of the agent is degraded during manufacture.

Hot melt extrusion can be used to prepare the drug eluting components. Single-screw or twin-screw systems can be used. As noted, if it is desired that the size of the particles be maintained during and after blending, carrier polymers should be used which can be melted at temperatures which do not degrade the agent. Otherwise, temperatures can be used which melt both the polymer and the agent.

Melting and casting can also be used to prepare the drug eluting components. The carrier polymer and agent, and any other desired components, are mixed together. The carrier polymer is melted and the melt is mixed so that the agent particles are evenly distributed in the melt, poured into a mold, and allowed to cool.

Solvent casting can also be used to prepare the drug eluting components. The polymer is dissolved in a solvent, and particles of agent are added. If the size of the agent particles are to be maintained, a solvent should be used which does not dissolve the agent particles, so as to avoid altering the size characteristics of the particles; otherwise, a solvent which dissolves both the polymer and agent particles can be used. The solvent-carrier polymer-agent particle mixture is then mixed to evenly distribute the particles (or thoroughly mix the solution), poured into a mold, and the solvent is evaporated.

### Manufacture/Assembly of System

In some embodiments, once the retention members of the gastric residence system have been affixed to the elastomeric component, the system is ready to be folded into its compacted configuration and placed into a capsule for storage, transport, and eventual administration. The system can be folded in an automated mechanical process, or by hand, and placed into a capsule of the appropriate size and material. More detail regarding manufacture and assembly of gastric residence systems, and of packaging the gastric residence system into capsules, can be found in International Patent Application Nos. WO 2015/191920, WO 2015/191925, WO 2017/070612, WO 2017/100367, WO 2017/205844, and WO 2018/227147.

### Methods of Treatment Using the Gastric Residence Systems

The gastric residence systems can be used to treat conditions requiring administration of a drug or agent over an extended period of time. In one embodiment, a gastric residence system is administered to a human. For long-term administration of agents or drugs which are taken for months, years, or indefinitely, administration of a gastric residence system periodically, such as once weekly or once every two weeks can provide substantial advantages in patient compliance and convenience. Accordingly, the gastric residence systems of the invention can be administered once every three days, once every five days, once weekly, once every ten days, or once every two weeks. The administration frequency is timed to coincide with the designed gastric residence period of the gastric residence system which is administered, so that at about the same time that a gastric residence system passes out of the stomach after its residence period, a new gastric residence system is administered.

Once a gastric residence system has been administered to a patient, the system provides sustained release of agent or drug over the period of gastric retention. After the period of gastric retention, the system degrades and passes out of the stomach. Thus, for a system with a gastric retention period of one week, the patient will swallow (or have administered to the stomach via other methods) a new system every week. Accordingly, in one embodiment, a method of treatment of a patient with a gastric retention system of the invention having a gastric residence period of a number of days D (where D-days is the gastric residence period in days), over a total desired treatment period T-total (where T-total is the desired length of treatment in days) with the agent or drug in the system, comprises introducing a new gastric residence system every D-days into the stomach of the patient, by oral administration or other methods, over the total desired treatment period. The number of gastric residence systems administered to the patient will be (T-total) divided by (D-days). For example, if treatment of a patient for a year (T-total=365 days) is desired, and the gastric residence period of the system is 7 days (D-days=7 days), approximately 52 gastric residence systems will be administered to the patient over the 365 days, as a new system will be administered once every seven days.

Alternatively, the patient can swallow (or have administered to the stomach via other methods) a new gastric residence system at the end of the effective release period of the gastric residence system. The “effective release period” or “effective release time” is the time over which the gastric residence system releases an effective amount of the agent contained in the system. Accordingly, in one embodiment, a method of treatment of a patient with a gastric residence system of the invention having an effective release period of a number of days E (where E-days is the effective release period in days), over a total desired treatment period T-total (where T-total is the desired length of treatment in days) with the agent in the system, comprises introducing a new gastric residence system every E-days into the stomach of the patient, by oral administration or other means, over the total desired treatment period. The number of gastric residence systems administered to the patient will be (T-total) divided by (E-days). For example, if treatment of a patient for a year (T-total=365 days) is desired, and the effective release period of the system is 7 days (E-days=7 days), approximately 52 gastric residence systems will be administered to the patient over the 365 days, as a new system will be administered once every seven days.

### Testing Methods

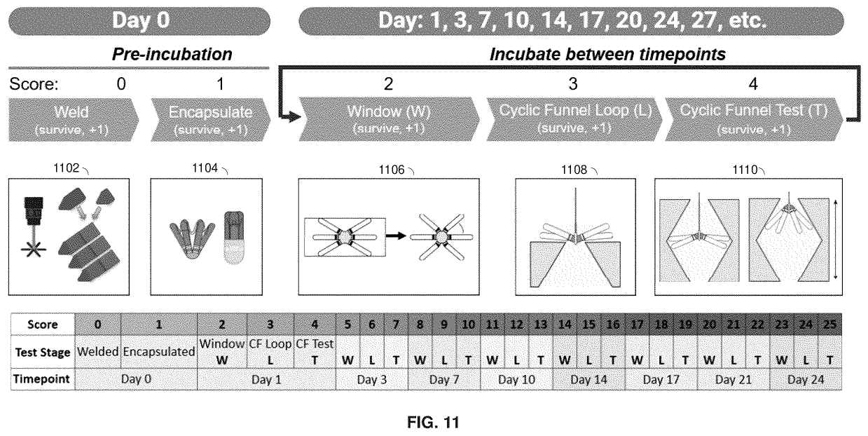

The Window-Cyclic Funnel (W-CF) test may be used to test the strength of a laser welded gastric residence system. The steps of the W-CF test are illustrated in FIG. 11. A gastric residence system is assembled using the laser welding techniques described herein at step 1102. The gastric residence system is allowed to fully cool and crystallize for 24 hours. The gastric residence system is then encapsulated at step 1104 and incubated in simulated fasted state gastric fluid for one day. Next, the gastric residence system is placed into a “window” fixture configured to hold the gastric residence system in a compressed position at step 1106. The window fixture compresses the gastric residence system for 4 hours while submerged in simulated fasted state gastric fluid at 37° C. The gastric residence system is then placed through a loop comprising a narrow (˜25 mm) orifice at step 1108. The gastric residence system is subsequently put through a cyclic funnel test for 100 cycles at step 1110. During the cyclic funnel test, the gastric residence system is gripped at its center by the loop, which is attached to a linear actuator. The gastric residence system is repeatedly and drawn up and down in the encapsulation and reverse-encapsulation directions within cone-shaped cavities. If the gastric residence system does not break during this process, the gastric residence system is re-incubated at 37° C. in simulated fasted state gastric fluid for two to three days, and the entire procedure is repeated. The stage at which the gastric residence system breaks is used to quantify the durability of the welds of the gastric residence system. The quantification system for the W-CF test is illustrated at the bottom of FIG. 11. For example, if a gastric residence system survives until Day 3 of the test and breaks during the window portion, the system receives a score of 5; whereas if the system survives until Day 7 of the test and breaks during the window portion, the system receives a score of 8.

Visual inspection may also be used to evaluate the adequacy of the welds of a gastric residence system. The retention members of the gastric residence system are inspected on all sides to determine whether each welded interface was fully melted from the top of the interface all the way through to the bottom. The interfaces are inspected for evidence of material flow and intermixing at each interface. Furthermore, the interfaces may be examined for visual indications of phase separation in a polymer blend. An adequately welded gastric residence system will have minimal phase separation. The gastric residence system is also inspected for burns or discolorations. The gastric residence system fails visual inspection if there are any burns or discolorations. The gastric residence system also fails visual inspection if there are any voids of material along a welded interface. The laser welding holder is also visually inspected. The gastric residence system fails visual inspection if any particulates or fibers become stuck to the silicone skin which coats the grooves of the laser welding holder.

Other techniques may be used to evaluate the adequacy of welds in a gastric residence system post-production. For instance, high-resolution imaging may be used to detect defects in the gastric residence system and perform dimensional analysis. Thermal imaging (e.g., using an infrared camera) may be used to measure the temperature of welds in the gastric residence system. Spectroscopy (e.g., using a near-infrared probe) may be used to acquire spectra across weld interfaces or across the entire gastric residence system. Additional evaluations may include acoustic testing to measure the change in transmission across weld interfaces, or scanning electron microscopy (SEM) to evaluate the structure of the gastric residence system.

Drug release may be measured using a USP II Dissolution apparatus. A gastric residence system is incubated at 37° C. in 900 mL of simulated fasted state gastric fluid. The apparatus provides agitation at 50 rpm. The amount of drug released is subsequently quantified using high pressure liquid chromatography (HPLC).

The presence of impurities or degradants in a gastric residence system may be measured using HPLC methods. The HPLC methods used may be specific to the active pharmaceutical ingredient contained in the gastric residence system. For example, HPLC-UV may be used for certain active pharmaceutical ingredients. The pass/fail criteria for HPLC evaluation may be specific to the active pharmaceutical ingredient and/or dosage in the gastric residence system.

### EXAMPLES

This invention is further illustrated by the following non-limiting examples.

### Example 1: Preparation of Gastric Residence Systems

Gastric residence systems were prepared according to the configuration described herein with respect to FIG. 10. The compositions of the drug eluting and non-drug eluting retention members are provided in Table 1 below.

TABLE 1

| Drug Eluting Retention Member | | Non-Drug Eluting Retention Members | |
|---|---|---|---|
| Component | Composition | Component | Composition |
| First inactive | 100% PCL | First inactive | 100% PCL |
| component | | component | |
| Time-dependent | 44.95% PCL12 | Time-dependent | 44.95% PCL12 |
| disintegrating matrix | 2.0% PEO100K | disintegrating matrix | 2.0% PEO100K |
| component | 53.0% 50/50 DL- | component | 53.0% 50/50 DL- |
| | Lactide/Glycolide | | Lactide/Glycolide |
| | copolymer, acid | | copolymer, acid |
| | (PDLG 5002A) | | (PDLG 5002A) |
| | 0.05% ferrosoferric | | 0.05% ferrosoferric |
| | oxide | | oxide |
| Second inactive | 70% Corbion PC17 | Second inactive | 70% Corbion PC17 |
| component | 30% bismuth | component | 30% bismuth |
| | subcarbonate | | subcarbonate |
| Enteric | 41.5% PCL | Enteric | 41.5% PCL |
| disintegrating matrix | 56.5% HPMCAS- | disintegrating matrix | 56.5% HPMCAS- |
| component | MG | component | MG |
| | 2.0% P407 | | 2.0% P407 |
| Third inactive | 70% Corbion PC17 | Third inactive | 70% Corbion PC17 |
| component | 30% bismuth | component | 30% bismuth |
| | subcarbonate | | subcarbonate |
| Drug eluting | 35.0% risperidone | Fourth inactive | 66.45% Corbion |
| component | 55.9% PCL17 | component | PC17 |
| | 5.0% VA64 | | 32.00% VA64 |
| | 3.0% P407 | | 1.50% P407 |
| | 0.5% Vit. E | | 0.05% FD&C Blue 1 |
| | succinate | | Alum Lake (11- |
| | 0.5% SiO₂ | | 13%) |
| | 0.05% FD&C | | |
| | Yellow 5 Alum Lake | | |
| | (16-18%) | | |
| | 0.05% FD&C Blue 1 | | |
| | Alum Lake (11- | | |
| | 13%) | | |
| Fourth inactive | 66.45% Corbion | — | — |
| component | PC17 | | |
| | 32.00% VA64 | | |
| | 1.50% P407 | | |
| | 0.05% FD&C Blue 1 | | |
| | Alum Lake (11- | | |
| | 13%) | | |

The radial lengths for each component of the retention members are provided in Table 2 below. The diameter of the gastric residence system, at its widest (i.e., from the tip of one retention member to the tip of an opposite retention member, including the elastomeric component), is about 46 mm in its unfolded state.

TABLE 2

| Drug Eluting Retention Member | | Non-Drug Eluting Retention Members | |
|---|---|---|---|
| | Radial | | Radial |
| Component | Length | Component | Length |
| First inactive | 1.3 mm | First inactive | 1.3 mm |
| component | | component | |
| Time-dependent | 1.0 mm | Time-dependent | 1.0 mm |
| disintegrating | | disintegrating | |
| matrix | | matrix | |
| component | | component | |
| Second inactive | 0.5 mm | Second inactive | 0.5 mm |
| component | | component | |
| Enteric | 1.85 mm | Enteric | 1.85 mm |
| disintegrating | | disintegrating | |
| matrix | | matrix | |
| component | | component | |
| Third inactive | 0.5 mm | Third inactive | 0.5 mm |
| component | | component | |
| Drug eluting | 7.3 mm | Fourth inactive | 13.1 mm |
| component | | component | |
| Fourth inactive | 5.8 mm | — | — |
| component | | | |

The gastric residence systems were assembled according to the laser welding techniques described herein. Gastric residence systems were assembled using one of two different laser systems, both operated at a wavelength of 1940 nm. The operating parameters for each laser system are provided in tables 3 and 4 below.

TABLE 3
Single Laser Scan Head with 1 mm Gaussian Beam

| Path Diameter | 20 (Hex) | 20 (Hex) | 15 (Circle) | 12 (Circle) |
|---|---|---|---|---|
| (mm) and Type | | | | |
| (Circle or Hex) | | | | |
| Passes | 400 | 260 | 250 | 230 |
| Power (%) | 26 | 26 | 55.5 | 50.5 |
| Speed (mm/s) | 2000 | 1750 | 3000 | 2800 |
| Wobble | 3000 Hz, 3 × 3 mm | | | |
| Weld | 2/20 | | | |
| Delay/Hold | | | | |
| Time (s) | | | | |

TABLE 4
Dual Laser Scan Heads with 3 mm flat top beam

| Path Diameter (mm) and | 14 (Circle) | 18.5 (Circle) | 20 (Circle) | PAUSE | 38.6 (Hex) |
|---|---|---|---|---|---|
| Type (Circle or Hex) | | | | 10 (Circle) | |
| Passes | 65 | 30 | 110 | 318 | 53 |
| Power (%) | 95 | 95 | 95 | 0 | 70 |

| | | | |
|---|---|---|---|
| Speed (mm/s) | 3000 | 1000 | 3000 |

| | |
|---|---|
| Wobble | None |
| Weld Delay/Hold Time (s) | 2/10* |

*With PAUSE, total hold time is 20 seconds. 10 seconds before Hex welds, and 10 seconds after.

### Example 2: Tensile Testing of Laser Welded Gastric Residence Systems

The tensile strength of laser welded gastric residence systems was compared to gastric residence systems prepared using infrared welding techniques, in which components are irradiated with infrared energy which is less focused than the energy provided by laser welding. Gastric residence systems wherein the drug eluting component comprised one of memantine (denoted as M116) or donepezil (denoted as DN34) were prepared using laser welding and infrared welding. The formulations of the drug eluting components for the M116-containing systems and DN34-containing systems are shown below in tables 5 and 6.

TABLE 5
M116-Containing Drug Eluting Arm Composition

| Component | % by weight |
|---|---|
| Memantine | 45 |
| Polycaprolactone | 41.9 |
| Poloxamer P407 | 2 |
| Poly(DL-lactic acid) | 10 |
| Alpha tocopherol | 0.5 |
| Silica | 0.5 |
| Sunset yellow | 0.1 |

TABLE 6
DN34-Containing Drug Eluting Arm Composition

| Component | % by weight |
|---|---|
| Donepezil | 40 |
| Polycaprolactone | 43.9 |
| Poloxamer P407 | 5 |
| Poly(DL-lactic acid) | 10 |
| Alpha tocopherol | 0.5 |
| Silica | 0.5 |
| FD&C Red #6 Barium Lake | 0.1 |

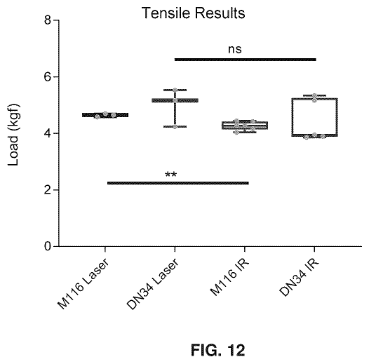

The tensile strength of each gastric residence system was tested using a version of the test described in ASTM D638 adapted for samples having a cross-section in the shape of an equilateral triangle with side lengths of 3.3 mm. As shown in FIG. 12, the tensile strength of the samples produced using laser welding and infrared welding were relatively similar. The tensile strength of the samples produced using laser welding was slightly stronger than the tensile strength of those produced using infrared welding techniques. Thus, the strength of laser welded gastric residence systems is equivalent or stronger than those assembled using infrared welding.

### Example 3: Laser Energy Absorption of Gastric Residence System Components

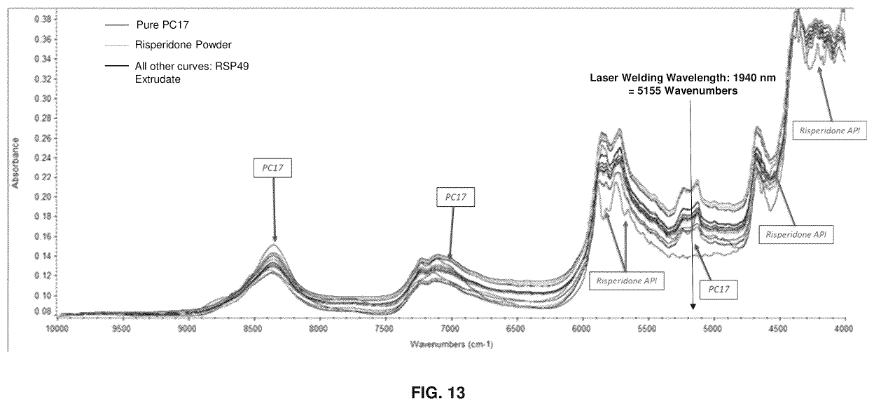

The absorbance of various components of gastric residence systems was quantified using a ThermoFisher Antaris II FT-NIR spectrometer in reflectance mode. The absorbance of the drug eluting component described above in Example 1 (“RSP49”), pure PCL (PC17), and risperidone powder were tested. For each material tested, rodstock samples were stacked together to completely cover the aperture of the spectrometer during testing. As shown in FIG. 13, each sample tested exhibited broad absorption in the near infrared range (defined as approximately 800-2500 nm, or 125000-4000 wavenumbers). In particular, pure PCL exhibited strong absorption across the near infrared range, with notable peaks around 8300 wavenumbers, 7100 wavenumbers, 5800 wavenumbers, 5100 wavenumbers, 4700 wavenumbers, and 4400 wavenumbers. As mentioned above with reference to Example 1, the wavelength of the laser used to laser weld the gastric residence systems is 1940 nm, or 5155 wavenumbers. PCL, as well as the other components tested, exhibited an absorbance peak near that point. Thus, the components of the gastric residence system exhibit strong absorbance of laser energy at the wavelengths contemplated herein.

### Example 4: Laser Energy Absorption of Gastric Residence System Components

The absorbance of the fourth inactive component described above with reference to Example 1 was also tested using the spectrometry methods described above with reference to Example 3. Absorbance was tested at various material thicknesses ranging from 0.50 mm to 4.00 mm. As shown in FIG. 14, the fourth inactive component exhibited broad absorbance across the near-infrared region with absorbance peaks at similar wavenumbers as those shown in FIG. 13, likely due to the presence of PCL in the fourth inactive component. The fourth inactive component also exhibited an additional absorbance peak at approximately 5000 wavenumbers, which is likely due to the addition of colorant (FD&C Blue 1 Alum Lake (11-13%). Thus, adding colorants or other excipients to the components of the gastric residence systems described herein may impact absorbance of laser energy.

### Example 5: Window-Cyclic Funnel (W-CF) Testing

The weld strength of laser welded gastric residence systems was assessed using the W-CF test. Once the components of the systems were laser welded together, the gastric residence systems were allowed to fully cool and crystallize for 24 hours. The systems were then encapsulated and incubated in simulated fasted state gastric fluid for one day. The systems were placed into “window” fixtures configured to hold the gastric residence systems in a compressed position for 4 hours while submerged in simulated fasted state gastric fluid at 37° C. The systems were then each placed through a loop comprising a narrow (˜25 mm) orifice. The systems were subsequently put through a cyclic funnel test for 100 cycles, during which the systems were gripped at their center by the loop, which is attached to a linear actuator, and drawn up and down in the encapsulation and reverse-encapsulation directions within cone-shaped cavities. If the gastric residence system did not break during this process, the gastric residence system was re-incubated at 37° C. in simulated fasted state gastric fluid for two to three days, and the entire procedure was repeated until breakage occurred.

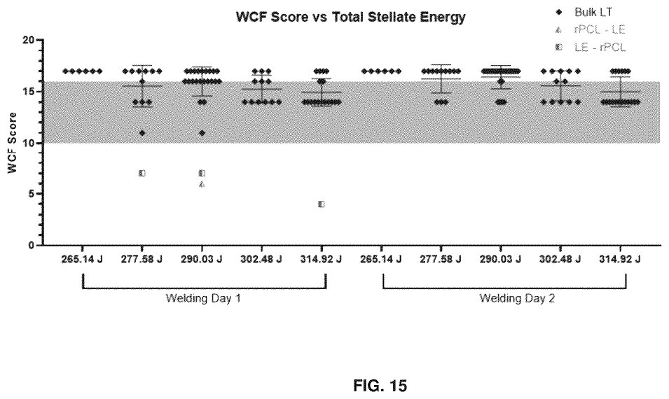

FIG. 15 shows the results of W-CF testing. Each sample was put through the W-CF test described above. The desired failure mode was defined as “bulk LT” over a score of 10, meaning that the desired failure mode was breakage of the time-dependent disintegrating matrix component after day 7. The scoring system for the W-CF test is shown in FIG. 11. As shown in FIG. 15, the W-CF score was measured for samples which received varying amounts of energy from the laser. At each total stellate energy tested, bulk LT exceeded 10, indicating strong links between laser welded components. A minority of stellates tested experienced failure modes other than failure of the “bulk LT” (time-dependent disintegrating matrix component). For example, some stellates experienced breakage at the interface between the second inactive component and the enteric disintegrating matrix component (rPCL-LE) or the interface between the enteric disintegrating matrix component and the third inactive component (LE-rPCL).

### Example 6: Melting Temperature of Gastric Residence System Components

The melting temperatures and pressures of the components of the gastric residence system of Example 1 are shown in FIG. 16. As shown in FIG. 18, the melting temperatures and pressures of the components vary, with the time-dependent disintegrating matrix component on the low end at 62-68° C. and the enteric disintegrating matrix component on the high end at 149-159° C. Components which are welded directly to one another in the order shown in FIG. 10 have melting temperatures within less than about 50° C. of one another. For example, the melting temperature of the time-dependent disintegrating matrix component is within 42-48° C. of the melting temperature of the second inactive component; the melting temperature of the second inactive component is within 39-49° C. of the melting temperature of the enteric disintegrating matrix component; the melting temperature of the enteric disintegrating matrix component is within 39-49° C. of the melting temperature of the third inactive component; the melting temperature of the third inactive component is within 10-20° C. of the melting temperature of the drug eluting component and within 16-33° C. of the melting temperature of the fourth inactive component; and the melting temperature of the drug eluting component is within less than 23° C. of the melting temperature of the fourth inactive component.

### EXEMPLARY EMBODIMENTS

Embodiment 1. A gastric residence system comprising: one or more retention members comprising: at least one drug eluting component; and at least one laser linker component laser welded to the at least one drug eluting component.

Embodiment 2. The gastric residence system of embodiment 1, wherein the one or more retention members are attached to an elastomeric component.

Embodiment 3. The gastric residence system of embodiment 2, wherein the elastomeric component is overmolded onto a first portion of at least one intercomponent anchor.

Embodiment 4. The gastric residence system of embodiment 3, wherein a laser linker component is overmolded onto a second portion of the at least one intercomponent anchor.

Embodiment 5. The gastric residence system of embodiment 4, wherein a first retention member is attached to the elastomeric component via the overmolded laser linker component.

Embodiment 6. The gastric residence system of any one of embodiments 1-5, wherein the at least one laser linker component comprises an inactive component.

Embodiment 7. The gastric residence system of embodiment 6, wherein the inactive component comprises polycaprolactone.

Embodiment 8. The gastric residence system of embodiment 7, wherein the inactive component further comprises bismuth subcarbonate.

Embodiment 9. The gastric residence system of embodiment 7 or 8, wherein the inactive component further comprises copovidone.

Embodiment 10. The gastric residence system of any one of embodiments 7-9, wherein the inactive component further comprises a poloxamer.

Embodiment 11. The gastric residence system of any one of embodiments 7-10, wherein the inactive component further comprises a colorant.

Embodiment 12. The gastric residence system of any one of embodiments 1-11, wherein the at least one laser linker component comprises an enteric disintegrating matrix.

Embodiment 13. The gastric residence system of embodiment 12, wherein the enteric disintegrating matrix comprises polycaprolactone.

Embodiment 14. The gastric residence system of embodiment 13, wherein the enteric disintegrating matrix further comprises HPMCAS.

Embodiment 15. The gastric residence system of embodiment 13 or 14, wherein the enteric disintegrating matrix further comprises a poloxamer.

Embodiment 16. The gastric residence system of any one of embodiments 1-15, wherein the at least one laser linker component comprises a time-dependent disintegrating matrix.

Embodiment 17. The gastric residence system of embodiment 16, wherein the time-dependent disintegrating matrix comprises polycaprolactone.

Embodiment 18. The gastric residence system of embodiment 17, wherein the time-dependent disintegrating matrix further comprises poly(ethylene oxide).

Embodiment 19. The gastric residence system of embodiment 17 or 18, wherein the time-dependent disintegrating matrix further comprises 50/50 DL-Lactide/Glycolide copolymer.

Embodiment 20. The gastric residence system of any one of embodiments 17-19, wherein the time-dependent disintegrating matrix further comprises ferrosoferric oxide.

Embodiment 21. The gastric residence system of any one of embodiments 1-20, wherein the drug eluting component comprises polycaprolactone.

Embodiment 22. The gastric residence system of embodiment 21, wherein the drug eluting component further comprises an active pharmaceutical ingredient.

Embodiment 23. The gastric residence system of embodiment 22, wherein the active pharmaceutical ingredient comprises one or more of meloxicam, escitalopram, citalopram, clopidogrel, prednisone, aripiprazole, risperidone, buprenorphine, naloxone, montelukast, memantine, digoxin, tamsulosin, ezetimibe, colchicine, loratadine, cetirizine, loperamide, omeprazole, entecavir, doxycycline, ciprofloxacin, azithromycin, antimalarial agents, levothyroxine, methadone, varenicline, contraceptives, stimulants, or nutrients.

Embodiment 24. The gastric residence system of any one of embodiments 21-23, wherein the drug eluting component further comprises copovidone.

Embodiment 25. The gastric residence system of any one of embodiments 21-24, wherein the drug eluting component further comprises a poloxamer.

Embodiment 26. The gastric residence system of any one of embodiments 21-25, wherein the drug eluting component further comprises vitamin E succinate.

Embodiment 27. The gastric residence system of any one of embodiments 21-26, wherein the drug eluting component further comprises silicon dioxide.

Embodiment 28. The gastric residence system of any one of embodiments 21-27, wherein the drug eluting component further comprises a colorant.

Embodiment 29. The gastric residence system of any one of embodiments 1-28, wherein a difference between a melt flow index of the at least one laser linker component and a melt flow index of the at least one drug eluting component is greater than 10%.

Embodiment 30. The gastric residence system of any one of embodiments 1-28, wherein a difference between a melt flow index of the at least one laser linker component and a melt flow index of the at least one drug eluting component is less than 50%.

Embodiment 31. The gastric residence system of any one of embodiments 1-30, wherein a polycaprolactone content of the at least one laser linker component is at least 30% by weight.

Embodiment 32. The gastric residence system of any of embodiments 1-30, wherein a polycaprolactone content of the at least one laser linker component is at least 40% by weight.

Embodiment 33. The gastric residence system of any of embodiments 1-32, wherein a polycaprolactone content of the at least one drug eluting component is at least 30% by weight.

Embodiment 34. The gastric residence system of any of embodiments 1-32, wherein a polycaprolactone content of the at least one drug eluting component is at least 40% by weight.

Embodiment 35. The gastric residence system of any of embodiments 1-34, wherein a polycaprolactone content of the at least one laser linker component is less than 50% by weight.

Embodiment 36. The gastric residence system of any of embodiments 1-34, wherein a polycaprolactone content of the at least one laser linker component is less than 65% by weight.

Embodiment 37. The gastric residence system of any of embodiments 1-34, wherein a polycaprolactone content of the at least one laser linker component is less than 75% by weight.

Embodiment 38. The gastric residence system of any of embodiments 1-37, wherein a polycaprolactone content of the at least one drug eluting component is less than 50% by weight.

Embodiment 39. The gastric residence system of any of embodiments 1-37, wherein a polycaprolactone content of the at least one drug eluting component is less than 65% by weight.

Embodiment 40. The gastric residence system of any of embodiments 1-37, wherein a polycaprolactone content of the at least one drug eluting component is less than 75% by weight.

Embodiment 41. The gastric residence system of any of embodiments 1-40, wherein a melting temperature of the at least one drug eluting component is within 1-75° C. of a melting temperature of the at least one laser linker component.

Embodiment 42. The gastric residence system of any of embodiments 1-40, wherein a melting temperature of the at least one drug eluting component is within 5-50° C. of a melting temperature of the at least one laser linker component.

Embodiment 43. The gastric residence system of any one of embodiments 1-42, wherein a width of a melt zone between laser welded components of the one or more retention members is between 0.5 mm and 5 mm.

Embodiment 44. The gastric residence system of any one of embodiments 1-42, wherein a width of a melt zone between laser welded components of the one or more retention members is between 1 mm and 3 mm.

Embodiment 45. The gastric residence system of any one of embodiments 1-44, wherein a depth of a melt zone between laser welded components of the one or more retention members is at least 90% of a depth of an interface between the laser welded components.

Embodiment 46. The gastric residence system of any one of embodiments 1-44, wherein a depth of a melt zone between laser welded components of the one or more retention members is at least 95% of a depth of an interface between the laser welded components.

Embodiment 47. The gastric residence system of any one of embodiments 1-46, wherein the gastric residence system receives a score of at least 7 when using the window-cyclic funnel test.

Embodiment 48. The gastric residence system of any one of embodiments 1-46, wherein the gastric residence system receives a score of at least 10 when using the window-cyclic funnel test.

Embodiment 49. The gastric residence system of any one of embodiments 1-48, wherein the gastric residence system is configured to be in a stressed configuration during administration and is configured to assume an open configuration when in a patient's stomach.

Embodiment 50. A method of making a gastric residence system comprising one or more retention members comprising at least one drug eluting component and at least one laser linker component, the method comprising: laser welding the at least one drug eluting component to the at least one laser linker component.

Embodiment 51. The method of embodiment 50, further comprising: placing the at least one laser linker component and the at least one drug eluting component in a laser welding holder prior to laser welding.

Embodiment 52. The method of embodiment 50 or 51, further comprising: applying a radial force which presses the at least one drug eluting component against the at least one laser linker component.

Embodiment 53. The method of embodiment 52, wherein the radial force is between 5 N and 200 N.

Embodiment 54. The method of embodiment 52, wherein the radial force is between 10 N and 50 N.

Embodiment 55. The method of any one of embodiments 51-54, further comprising: applying a downward force which presses the at least one drug eluting component and the at least one laser linker component against the laser welding holder.

Embodiment 56. The method of embodiment 55, wherein the vertical force is between 100 N and 5000 N.

Embodiment 57. The method of embodiment 55, wherein the vertical force is between 200 N and 2800 N.

Embodiment 58. The method of any one of embodiments 50-57, wherein laser welding is performed using a laser having a wavelength between 0.7 μm and 2.5 μm.

Embodiment 59. The method of any one of embodiments 50-57, wherein laser welding is performed using a laser having a wavelength between 1.9 μm and 2.0 μm.

Embodiment 60. The method of any one of embodiments 50-59, wherein laser welding is performed using a laser having a Gaussian energy profile or a top hat energy profile.

Embodiment 61. The method of any one of embodiments 50-60, wherein laser welding is performed using a laser having a beam diameter between 0.5 mm and 5 mm.

Embodiment 62. The method of any one of embodiments 50-60, wherein laser welding is performed using a laser having a beam diameter between 1 mm and 3 mm.

Embodiment 63. The method of any one of embodiments 1-62, wherein laser welding is performed in an environment with less than 40% humidity.

Embodiment 64. The method of any one of embodiments 1-62, wherein laser welding is performed in an environment with less than 25% humidity.

Embodiment 65. The method of any one of embodiments 1-64, wherein laser welding is performed in an environment with at least 10% humidity.

Embodiment 66. The method of any one of embodiments 1-64, wherein laser welding is performed in an environment with at least 15% humidity.

Embodiment 67. The method of any one of embodiments 50-66, further comprising: laser welding the one or more retention members to an elastomeric component, wherein the elastomeric component comprises one or more laser linker components configured to be laser welded to the one or more retention members.

Embodiment 68. The method of embodiment 67, wherein the gastric residence system comprises at least two retention members laser welded to the elastomeric component.

Embodiment 69. The method of embodiment 67, wherein the gastric residence system comprises at least three retention members laser welded to the elastomeric component.

Embodiment 70. The method of embodiment 67, wherein the gastric residence system comprises at least four retention members laser welded to the elastomeric component.

Embodiment 71. The method of embodiment 67, wherein the gastric residence system comprises at least five retention members laser welded to the elastomeric component.

Embodiment 72. The method of embodiment 67, wherein the gastric residence system comprises at least six retention members laser welded to the elastomeric component.

Embodiment 73. The method of any of embodiments 68-72, wherein laser welding the at least one drug eluting component to the at least one laser linker component comprises laser welding along a repetitive path connecting corresponding interfaces between the at least one drug eluting component and the at least one linker on each retention member.

Embodiment 74. The method of embodiment 73, wherein the repetitive path is a circular path.

Embodiment 75. The method of any of embodiments 50-72, wherein laser welding the at least one drug eluting component to the at least one laser linker component comprises laser welding back and forth along an interface between the at least one drug eluting component and the at least one laser linker component.

Embodiment 76. A laser welding system comprising: a laser welding holder configured to hold pieces of a gastric residence system in an assembled order; at least one radial pressure piston configured to apply a radial force to the gastric residence system in a direction transverse to seams between each piece; at least one vertical pressure piston configured to apply a downward force to the gastric residence system in a direction parallel to the seams between each piece; and at least one laser configured to weld the seams between each piece of the gastric residence system.

Embodiment 77. The laser welding system of embodiment 76, wherein the laser welding holder comprises aluminum or stainless steel.

Embodiment 78. The laser welding system of embodiment 76 or 77, wherein the laser welding holder is plated with nickel.

Embodiment 79. The laser welding system of any one of embodiments 76-78, wherein the laser welding holder comprises grooves sized and shaped to receive the pieces of the gastric residence system.

Embodiment 80. The laser welding system of any one of embodiments 76-79, wherein a top surface of the laser welding holder comprises a layer of silicone.

Embodiment 81. The laser welding system of any of embodiments 76-80, wherein a top surface of the laser welding holder comprises a layer of polytetrafluoroethylene-coated glass.

Embodiment 82. The laser welding system of any of embodiments 76-81, further comprising a cover configured to hold the components of the gastric residence system in place during laser welding.

Embodiment 83. The laser welding system of embodiment 82, wherein the cover comprises silicone or polytetrafluoroethylene-coated glass.

Embodiment 84. The laser welding system of embodiment 82 or 83, further comprising a top layer on top of the cover.

Embodiment 85. The laser welding system of embodiment 84, wherein the top layer comprises quartz glass.

Embodiment 86. The laser welding system of any one of embodiments 76-85, wherein the radial force applied by the at least one radial pressure piston is between 5 and 200 N.

Embodiment 87. The laser welding system of any one of embodiments 76-85, wherein the radial force applied by the at least one radial pressure piston is between 10 and 50 N.

Embodiment 88. The laser welding system of any one of embodiments 76-87, wherein the downward force applied by the at least one vertical pressure piston is between 100 and 5000 N.

Embodiment 89. The laser welding system of any one of embodiments 76-87, wherein the downward force provided by the at least one vertical pressure piston is between 200 and 2800 N.

Embodiment 90. The laser welding system of any one of embodiments 76-89, wherein the at least one laser has a wavelength between 0.7 μm and 2.5 μm.

Embodiment 91. The laser welding system of any one of embodiments 76-89, wherein the at least one laser has a wavelength between 1.9 μm and 2.0 μm.

Embodiment 92. The laser welding system of any one of embodiments 76-91, wherein the at least one laser has a Gaussian energy profile or a top hat energy profile.

Embodiment 93. The laser welding system of any one of embodiments 76-92, wherein the at least one laser has a beam diameter between 0.5 mm and 5 mm.

Embodiment 94. The laser welding system of any one of embodiments 76-92, wherein the at least one laser has a beam diameter between 1 mm and 3 mm.

Although the foregoing invention has been described in some detail by way of illustration and example for purposes of clarity of understanding, it is apparent to those skilled in the art that certain changes and modifications will be practiced. Therefore, the description and examples should not be construed as limiting the scope of the invention.

1. A gastric residence system comprising:
one or more retention members comprising:
at least one drug eluting component; and
at least one laser linker component laser welded to the at least one drug eluting component.

2. The gastric residence system of claim 1, wherein the one or more retention members are attached to an elastomeric component.

3. The gastric residence system of claim 2, wherein the elastomeric component is overmolded onto a first portion of at least one intercomponent anchor.

4. The gastric residence system of claim 3, wherein a laser linker component is overmolded onto a second portion of the at least one intercomponent anchor.

5. The gastric residence system of claim 4, wherein a first retention member is attached to the elastomeric component via the overmolded laser linker component.

6. The gastric residence system of claim 1, wherein the at least one laser linker component comprises an inactive component.

7. The gastric residence system of claim 1, wherein the at least one laser linker component comprises an enteric disintegrating matrix.

8. The gastric residence system of claim 1, wherein the at least one laser linker component comprises a time-dependent disintegrating matrix.

9. The gastric residence system of claim 1, wherein a difference between a melt flow index of the at least one laser linker component and a melt flow index of the at least one drug eluting component is at least 10%.

10. The gastric residence system of claim 1, wherein a difference between a melt flow index of the at least one laser linker component and a melt flow index of the at least one drug eluting component is less than 50%.

11. The gastric residence system of claim 1, wherein a polycaprolactone content of the at least one laser linker component is at least 30% by weight.

12. The gastric residence system of claim 1, wherein a polycaprolactone content of the at least one drug eluting component is at least 30% by weight.

13. The gastric residence system of claim 1, wherein a melting temperature of the at least one drug eluting component is within 1-75° C. of a melting temperature of the at least one laser linker component.

14. The gastric residence system of claim 1, wherein a width of a melt zone between laser welded components of the one or more retention members is between 0.5 mm and 5 mm.

15. The gastric residence system of claim 1, wherein a depth of a melt zone between laser welded components of the one or more retention members is at least 90% of a depth of an interface between the laser welded components.

16. A method of making a gastric residence system comprising one or more retention members comprising at least one drug eluting component and at least one laser linker component, the method comprising:
laser welding the at least one drug eluting component to the at least one laser linker component.

17. The method of claim 16, further comprising:
placing the at least one laser linker component and the at least one drug eluting component in a laser welding holder prior to laser welding.

18. The method of claim 16, further comprising:
applying a radial force which presses the at least one drug eluting component against the at least one laser linker component.

19. The method of claim 16, further comprising:
applying a downward force which presses the at least one drug eluting component and the at least one laser linker component against the laser welding holder.

20. The method of claim 16, wherein laser welding is performed using a laser having a wavelength between 0.7 μm and 2.5 μm.

21. The method of claim 16, wherein laser welding is performed using a laser having a Gaussian energy profile or a top hat energy profile.

22. The method of claim 16, wherein laser welding is performed using a laser having a beam diameter between 0.5 mm and 5 mm.

23. The method of claim 16, wherein laser welding is performed in an environment with less than 40% humidity.

24. The method of claim 16, wherein laser welding is performed in an environment with at least 10% humidity.

25. The method of claim 16, further comprising:
laser welding the one or more retention members to an elastomeric component, wherein the elastomeric component comprises one or more laser linker components configured to be laser welded to the one or more retention members.

26. The method of claim 16, wherein laser welding the at least one drug eluting component to the at least one laser linker component comprises laser welding along a repetitive path connecting corresponding interfaces between the at least one drug eluting component and the at least one linker on each retention member.

27. A laser welding system comprising:
a laser welding holder configured to hold pieces of a gastric residence system in an assembled order;
at least one radial pressure piston configured to apply a radial force to the gastric residence system in a direction transverse to seams between each piece;
at least one vertical pressure piston configured to apply a downward force to the gastric residence system in a direction parallel to the seams between each piece; and
at least one laser configured to weld the seams between each piece of the gastric residence system.

28. The laser welding system of claim 27, wherein the laser welding holder comprises grooves sized and shaped to receive the pieces of the gastric residence system.

29. The laser welding system of claim 27, further comprising a cover configured to hold the components of the gastric residence system in place during laser welding.

30. The laser welding system of claim 27, further comprising a top layer on top of the cover.
